(19) United States
(12) Patent Application Publication
(10) Pub. No.: US 2022/0323544 A1
(43) Pub. Date: Oct. 13, 2022

(54) SOLID COMPOSITIONS COMPRISING AN EGF(A) DERIVATIVE AND A SALT OF N-(8-(2-HYDROXYBENZOYL)AMINO)CAPRYLIC ACID

(71) Applicant: Novo Nordisk A/S, Bagsvaerd (DK)

(72) Inventors: Betty Lomstein Pedersen, Glostrup (DK); Birgitte Nissen, Ballerup (DK); Kaisa Naelapaeae, Copenhagen (DK); Andreas Vegge, Frederiksberg (DK)

(21) Appl. No.: 17/628,697

(22) PCT Filed: Aug. 7, 2020
(86) PCT No.: PCT/EP2020/072227

§ 371 Date: Jan. 20, 2022

(30) Foreign Application Priority Data
Aug. 7, 2019 (EP) 19190506.6

(51) Int. Cl. A61K 38/18; A61K 9/20; A61P 9/00

(52) U.S. Cl. CPC A61K 38/1808; A61K 9/2013; A61P 9/00; A61K 9/2027; A61K 9/2054

(57) ABSTRACT

The invention relates to pharmaceutical compositions comprising a PCSK9 inhibitor, such as an EGF(A) peptide, and a salt of N-(8-(2-hydroxybenzoyl)amino)caprylic acid. The invention further relates to processes for the preparation of such compositions, and their use in medicine.

### TECHNICAL FIELD OF THE INVENTION

The present invention relates to solid compositions comprising a PCSK9 inhibitor and a salt of N-(8-(2-hydroxybenzoyl)amino)caprylic acid, their method of preparation and their use in medicine.

### INCORPORATION-BY-REFERENCE OF THE SEQUENCE LISTING

The Sequence Listing, entitled “SEQUENCE LISTING”, is 4 KB and was created on Jul. 1, 2020 and is incorporated herein by reference.

### BACKGROUND

High LDL-C (Low Density Lipoprotein cholesterol) levels and dyslipidaemia are well-recognised drivers of cardiovascular disease.

Statins have been approved for the treatment of dyslipidemia for 25 years. This class has demonstrated substantial and consistent reduction of cardiovascular events with an acceptable safety profile. The best-selling statin, atorvastatin (Lipitor™) was the world's best-selling drug of all time, with more than $125 billion in sales from 1996 to 2012.

Despite the availability and widespread use of statins and other lipid lowering agents, many patients do not reach their target LDL-C levels and remain at high risk for developing cardiovascular disease. PCSK9 (Proprotein Convertase Subtilisin/Kexin type 9) promotes hepatic LDL-R (LDL receptor) degradation, thereby reducing hepatic LDL-R surface expression and consequently clearance of LDL particles. Conversely, blocking of PCSK9 increases the clearance of LDL-C as well as other atherogenic lipoproteins. Indeed, LDL receptors contribute to the clearance of atherogenic lipoproteins other than LDL, such as intermediate-density lipoproteins and remnant particles. Increased intermediate-density lipoproteins and remnant particle clearance may have therapeutic benefits beyond that provided by LDL reduction.

Statins increase the expression of both LDL-R and PCSK9 via the SREBP2 transcription factor. The increased expression of PCSK9 may diminish the effect of statins on LDL-C clearance from the circulation.

By inhibiting the binding of PCSK9 to the LDL-R and thereby preventing LDL-R degradation the efficacy of statins is enhanced. Taken together, PCSK9 inhibition offers a novel approach to lipid management.

Two anti-PCSK9 antibodies, alirocumab/Praluent® and evolocumab/Repatha®, have been approved for the treatment of high LDL-C levels. These are administered by 1 ml subcutaneous injections every two weeks.

The EGF(A) (Epidermal Growth Factor-like domain A) sequence (40 amino acids) of the LDL-R (LDL-R-(293-332)) is well recognized as the site for PCSK9 binding. The isolated wild-type EGF(A) peptide has been shown to inhibit the binding of PCSK9 to the LDL-R with an IC₅₀ in the low μM range (Biochemical and Biophysical Research Communications 375 (2008) 69-73). This poor potency has prevented a practical pharmaceutical use of the EGF(A) peptide. Furthermore, the half-life of such peptides would be expected to be too short to be of therapeutic use.

WO2012177741 and J. Mol. Biol. (2012) 422,685-696 disclose analogues of the EGF(A) and Fc-Fusion thereof.

Alternative EGF(A) peptide based PCSK9 inhibitors with an extended half-life have been disclosed in WO2017/121850. In order to increase the usability of such drugs it is of interest to develop a suitable oral formulation. Oral administration of therapeutic peptides is challenging due to the rapid degradation of such peptides in the gastrointestinal system.

Oral bioavailability of peptide compounds is generally limited but useful results have been obtained for semaglutide as described in WO 2012/080471 and WO 2013/139694.

### SUMMARY

The present invention in an aspect relates to a composition comprising a PCSK9 inhibitor and an absorption enhancer or delivery agent. The excipients of the composition according to the invention in an embodiment includes a very high content of the delivery agent and a minimal content of further excipients as described herein below. The provided compositions display an accelerated dissolution enabling fast uptake of the active pharmaceutical ingredient.

Described herein are pharmaceutical compositions demonstrating an accelerate dissolution and thus an improved exposure of the PCSK9 inhibitor by oral administration can be foreseen based on previous data obtained with semaglutide. The inventors have found that the dissolution of a PCSK9 inhibitor composition occurs faster when the composition is prepared with a very high content of the absorption enhancer and a minimal content of any further excipients.

In an aspect the invention relates to a composition wherein the weight ratio of the delivery agent relative to the total composition, or in particular, relative to the other excipients of the composition, is very high.

In one embodiment, the invention relates to a pharmaceutical composition comprising a PCSK9 inhibitor, a delivery agent and/or absorption enhancer such as SNAC, wherein the delivery agent/absorption enhancer constitutes at least 90%, such as at least 95% w/w, of the excipients of the composition.

In one embodiment the composition comprises:
a) 0.5-100 mg EGF(A) derivative,
b) 20-1000 mg, such as 50-600 mg, of a salt of N-(8-(2-hydroxybenzoyl)amino)caprylic acid and

wherein said salt of N-(8-(2-hydroxybenzoyl)amino)caprylic acid constitutes at least 90 w/w of the excipients of the composition.

In one embodiment, the invention relates to a pharmaceutical composition comprising a PCSK9 inhibitor, a delivery agent and/or absorption enhancer such as SNAC, wherein the delivery agent/absorption enhancer constitutes at least 70% w/w of the composition.

In additional embodiments, the composition further includes a lubricant.

In one embodiment the composition comprises:
a) 0.5-100 mg EGF(A) derivative,
b) 20-1000 mg, such as 50-600 mg, of a salt of N-(8-(2-hydroxybenzoyl)amino)caprylic acid and
c) 0.5-50 mg, such as 1-30 mg lubricant,

wherein said salt of N-(8-(2-hydroxybenzoyl)amino)caprylic acid constitutes at least 90% w/w of the excipients of the composition.

In an aspect the invention relates to a method of preparing a pharmaceutical composition as described herein such as a method comprising the steps of;
a) granulating a mixture comprising the delivery agent, the PCSK9 inhibitor and optionally a lubricant and
b) compressing the granulate obtained in step a) into tablets and optionally adding further lubricant to the granulate prior to compression.

In a further aspect the invention relates to a composition or a granule as defined herein for use in medicine, such as for improving lipid parameters and/or preventing and/or treating cardiovascular diseases , wherein said composition is administered orally.

In a further aspect the invention relates to a method of improving lipid parameters and/or preventing and/or treating cardiovascular diseases comprising administering the composition as defined herein to a patient in need thereof, wherein said composition is a tablet and is administered orally.

### BRIEF DESCRIPTION OF DRAWINGS

FIG. 1 shows fast dissolution of test compositions 2, 3, 5, 6 and 7 compared to test composition 1.

### DESCRIPTION

An aspect of the invention relates to a composition comprising a PCSK9 inhibitor and an absorption enhancer or delivery agent. The composition may be in the form suitable for oral administration, such as a tablet, sachet or capsule. In an embodiment the composition is an oral composition, or a pharmaceutical composition, such as an oral pharmaceutical composition.

The composition according to the invention in an embodiment includes a high content of the delivery agent and a minimal content of further excipients as described herein below. The provided compositions display an accelerated dissolution and thereby enableinga fast uptake of the active pharmaceutical ingredient.

### PCSK9 Inhibitor

The term “PCSK9 inhibitor” as used herein refers to a compound, which fully or partially prevents PCSK9 from binding to the human Low Density Lipoprotein Receptor (LDL-R).

The EGF(A) LDL-R(293-332) peptide binds PCSK9, but is not considered a PCSK9 inhibitor due to a relatively week binding to PCSK9. The potential of an EGF(A) analogue to inhibit PCSK9 may be measured in an ELISA assay (such as Assay I herein) providing the apparent affinity of the EGF(A) analogue or a compound comprising an EGF(A) analogue reported as a Kᵢ, A low Ki is thus characteristic for compounds with a strong inhibitory function as described in WO2017/121850. Based on their ability to inhibit the interaction of PCSK9 with LDL-R, such compounds are referred to as PCSK9 inhibitors. Based on the findings described in WO2017/121850 a suitable PCSK9 inhibitor has a Ki below 8 nM, such as below 5 nM. In one embodiment the PCSK9 inhibitor has a Ki around 0.5-8 nM, or such as 0.5-5 nM or such as 1.0-4 nM. An assay suited for determining the Ki is described herein in Assay I.

In one embodiment the PCSK9 inhibitor has an inhibitory function at least comparable to EGF(A) 301L. In one embodiment the PCSK9 inhibitor has an PCSK9 inhibitory function comparable to EGF(A) 301L. In a given assay, such as Assay I described herein, the ratio

$$\frac{Ki ( PCSK 9 inhibitor )}{Ki ( EG F ( A ) 301 L )}$$

is thus preferably below 2, such as below 1.5, such as below 1.2. In one embodiment the ratio is at most 1.0, such as at most 0.8, such as at most 0.7, such as at most 0.6 or such as at most 0.5. In one embodiment

$$\frac{Ki ( PCSK 9 inhibitor )}{Ki ( EG F ( A ) 301 L )}$$

the ratio is 2.0-0.2, such as 1.5-0.5 or such as 1.2-0.8.

In one embodiment where the PCSK9 inhibitor has an inhibitory function comparable to EGF(A) 301L. In one embodiment the PCSK9 inhibitor has an improved PCSK9 inhibitory function compared to EGF(A) 301L, 309R, 310K. In a given assay, such as Assay I described herein, the ratio

$$\frac{Ki ( PCSK 9 inhibitor )}{Ki ( EG F ( A ) 301 L , 3 \; 09 R , 312 E )}$$

is thus preferably below 2, such as below 1.5, such as below 1.2. In one embodiment the ratio is at most 1.0, such as at most 0.8, such as at most 0.7, such as at most 0.6 or such as at most 0.5. In one embodiment

$$\frac{Ki ( PCSK 9 inhibitor )}{Ki ( EG F ( A ) 301 L )}$$

the ratio is 2.0-0.2, such as 1.5-0.5 or such as 1.2-0.8.

In one embodiment PCSK9 inhibitor comprises an EGF(A) peptide analogue as further described below.

### EGF(A) Compound

The term “EGF(A) compound” is used herein to generally refer to a compound comprising an EGF(A) peptide, encompassing wt-LDL-R(293-332) as defined by SEQ ID NO: 1 and analogues hereof. The term EGF(A) compound encompasses derivatives of EGF-(A) peptide and analogue thereof i.e. EGF(A) peptide analogues with a substituent as described herein is a typical example of an EGF(A) compound.

### EGF(A) Peptides

The term “peptide”, as e.g. used in the context of the invention, refers to a compound which comprises a series of amino acids interconnected by amide (or peptide) bonds. In a particular embodiment the peptide consists of amino acids interconnected by peptide bonds.

The peptide of the invention comprises at least 35, such as 36, 37, 38, 39 or at least 40 amino acids. In a particular embodiment the peptide is composed of 36, such as 38 or 40 amino acids. In an additional particular embodiment, the peptide consists of 35, 36, 37, 38, 39 or 40 amino acids.

In the presence of amino acid additions, referred to herein as N-terminal and C-terminal elongations, the peptide of the invention may comprise up to 140 amino acids. In an embodiment, the peptide of the invention may comprise or consist of 41 amino acid residues. In a particular embodiment, the peptide comprises 40-140, 40-120, 40-100, 40-80, 40-60 or 40-50 amino acids.

The terms “EGF(A) domain of the LDL-R”, “LDL-R (293-332)”, “native LDL-R (293-332), “EGF(A) (293-332)”, “wild-type EGF(A)”, “wt-EGF(A)” or “native EGF(A)” as used herein refer to a peptide consisting of the sequence SEQ ID NO: 1.

### SEQ ID NO: 1 is:

Gly-Thr-Asn-Glu-Cys-Leu-Asp-Asn-Asn-Gly-Gly-Cys-Ser-His-Val-Cys-Asn-Asp-Leu-Lys-Ile-Gly-Tyr-Glu-Cys-Leu-Cys-Pro-Asp-Gly-Phe-Gln-Leu-Val-Ala-Gln-Arg-Arg-Cys-Glu.

In this formula the numbering of the amino acid residues follows the numbering for the EGF(A) domain of the LDL-R (LDL-R-(293-332)), wherein the first (N-terminal) amino acid residue is numbered or accorded position no. 293, and the subsequent amino acid residues towards the C-terminus are numbered 294, 295, 296 and so on, until the last (C-terminal) amino acid residue, which in the EGF(A) domain of the LDL-R is Glu with number 332.

The numbering is done differently in the sequence listing, where the first amino acid residue of SEQ ID NO: 1 (Gly) is assigned no. 1, and the last (Glu) no. 40. The same applies for the other sequences of the sequence listing, i.e. the N-terminal amino acid assigned is no. 1 irrespective of its positioning relative to 293Gly or 293 substituting amino acid residue by reference to LDL-R(293-332). However, herein the numbering of amino acid positions is with reference to LDL-R(293-332), as explained above.

The present invention relates to analogues of the EGF(A) peptide identified by SEQ ID NO:1 and derivatives of such EGF(A) peptide analogues of the wild-type EGF(A) domain of LDLR defined by SEQ ID NO: 1.

The term “analogue” generally refers to a peptide, the sequence of which has one or more amino acid changes when compared to a reference amino acid sequence.

The terms “analogue of the invention”, “peptide analogue of the invention”, “LDL-R(293-332) analogue”, “EGF(A) analogue” or “analogue of SEQ ID NO: 1” as used herein may be referred to as a peptide, the sequence of which comprises amino acid substitutions, i.e. amino acid replacement, relative to sequence SEQ ID NO: 1. An “analogue” may also include amino acid elongations in the N-terminal and/or C-terminal positions and/or truncations in the N-terminal and/or C-terminal positions.

The level of identity to SEQ ID NO.:1 can be calculated by determining the number of amino acids that are not changed relative to SEQ ID NO 1. SEQ ID NO: 1 consists of 40 amino acid residues and if three amino acid substitutions are introduced the level of identity is 37/40%=92.5%. If 5 amino acid residues are changed the level of identity is 87, 5%. If the peptide is N-terminal or C-terminal elongated that part is usually not included in the comparison, whereas a deletion of one or more amino acids shortens the comparator. For instance, in the examples above, if the N-terminal amino acid is deleted the level of identity is slightly reduced to 36/39X100% and 34/39X100%, respectively. When discussing identity of the back-bone sequence of a derivative the amino acid residue of the substituent e.g. the residue to which the substituent is attached, also termed the amino acid residue of the substituent, may be either a wild type (wt) or a substituted amino acid. If the amino acid residue of the substituent is a wild type residue, such as the N-term Gly or 312K this residue is included in the calculation of identity level, whereas a Lys in any other position from 293 to 332 would be an amino acid substitution and not included when calculated amino acid identity to SEQ ID NO.:1.

In one embodiment the EGF(A) peptide analogue has 1-15 amino acid substitutions compared to SEQ ID NO.: 1. In one embodiments the EGF(A) peptide analogue has 1-10 amino acid substitutions compared to SEQ ID NO.: 1. In one embodiments the EGF(A) peptide analogue has 1-8 amino acid substitutions compared to SEQ ID NO.: 1, such as 1-7, 1-6, 1-5 amino acid substitutions compared to SEQ ID NO.: 1. In a particular embodiment, up to 7 amino acid substitutions may be present, for example up to 6, 5, 4, 3, 2 or 1 amino acid substitutions may be present in the EGF-1 peptide analogue.

In one embodiment the analogue of the invention has at least 75% identity, such as 80%, such as 85, such as 90 or even 95% identity to SEQ ID NO.:1 corresponding to up to 10, 8, 6, 4 and 2 amino acid substitutions relative to SEQ ID NO 1, respectively in case of no truncation.

Each of the peptide analogues of the invention may be described by reference to i) the number of the amino acid residue in the native EGF(A) (LDL-R(293-332)) which corresponds to the amino acid residue which is changed (i.e., the corresponding position in native LDL-R(293-332) EGF(A)), and to ii) the actual change.

In other words, the peptide analogues of the invention may be described by reference to the native LDL-R(293-332) EGF(A) peptide, namely as a variant thereof in which a number of amino acid residues have been changed when compared to native LDL-R(293-332) EGF(A) (SEQ ID NO: 1). These changes may represent, independently, one or more amino acid substitutions.

The followings are non-limiting examples of suitable analogue nomenclature:

The EGF(A) peptide incorporated in the derivative of Example 2 in WO2017/121850 is thus referred to as the following LDL-R(293-332) EGF(A) analogue: (301Leu, 309Arg) LDL-R(293-332) EGF(A), or (Leu301, Arg309)-LDL-R(293-332) EGF(A) or (301L,309R) LDL-R(293-332) or (L301,R309) LDL-R(293-332). This means that when this analogue is aligned with native LDL-R(293-332), it has i) a Leu at the position in the analogue which corresponds, according to the alignment, to position 301 in native LDL-R(293-332) EGF(A), ii) an Arg at the position in the analogue which corresponds to position 309 in native LDL-R(293-332) EGF(A).

Analogues “comprising” certain specified changes may comprise further changes, when compared to SEQ ID NO: 1.

In a particular embodiment, the analogue “has” or “comprises” the specified changes. In a particular embodiment, the analogue “consists of” the changes. When the term “consists” or “consisting” is used in relation to an analogue e.g. an analogue consists or consisting of a group of specified amino acid substitutions, it should be understood that the specified amino acid substitutions are the only amino acid substitutions in the peptide analogue. In contrast an analogue “comprising” a group of specified amino acid substitutions may have additional substitutions.

As is apparent from the above examples, amino acid residues may be identified by their full name, their one-letter code, and/or their three-letter code. These three ways are fully equivalent.

The expressions “a position equivalent to” or “corresponding position” may be used to characterise the site of change in a variant LDL-R(293-332) EGF(A) sequence by reference to the reference sequence native LDL-R(293-332) EGF(A) (SEQ ID NO: 1). Equivalent or corresponding positions, as well as the number of changes, are easily deduced, e.g. by simple handwriting and eyeballing; and/or a standard protein or peptide alignment program may be used, such as “align” which is based on a Needleman-Wunsch algorithm.

In what follows, it may occur that a chemical formula is defined such that two subsequent chemical groups may both be selected to be “a bond”. In such instances, the two subsequent chemical groups would actually be absent, and just one bond would connect the surrounding chemical groups.

Amino acids are molecules containing an amino group and a carboxylic acid group, and, optionally, one or more additional groups, often referred to as a side chain.

The term “amino acid” includes proteinogenic (or natural) amino acids (amongst those the 20 standard amino acids), as well as non-proteinogenic (or non-natural) amino acids. Proteinogenic amino acids are those which are naturally incorporated into proteins. The standard amino acids are those encoded by the genetic code. Non-proteinogenic amino acids are either not found in proteins, or not produced by standard cellular machinery (e.g., they may have been subject to post-translational modification). Non-limiting examples of non-proteinogenic amino acids are Aib (α-aminoisobutyric acid, or 2-aminoisobutyric acid), norleucine, norvaline as well as the D-isomers of the proteinogenic amino acids.

In what follows, each amino acid of the peptides of the invention for which the optical isomer is not stated is to be understood to mean the L-isomer (unless otherwise specified).

### EGF(A) Peptide Analogues

An aspect of the invention relates to an analogue of a peptide of SEQ ID NO: 1. The peptide analogues of the invention may be defined as peptides comprising an amino acid sequence which is an analogue of SEQ ID NO: 1. The peptide analogues of the invention have the ability to bind to PCSK9. In a specific embodiment, the analogues of the invention have an improved ability to bind to PCSK9, for example compared to native LDL-R(293-332) (native EGF-(A)) or to other PCSK9-binding compounds.

The peptide analogues of the invention have the ability to inhibit PCSK9 binding to the LDL-R. In one embodiment the peptide is a PCSK9 inhibitor. In one embodiment the peptide inhibits PCSK9 binding to human Low Density Lipoprotein Receptor (LDL-R). Such binding may be assessed using the assay described in Assay IV herein. In one embodiment the peptide analogues and peptide derivatives of the invention are PCSK9 inhibitor peptides or simply PCSK9 inhibitors. In one embodiment the invention relates to a peptide analogue of SEQ ID NO.:1, wherein peptide analogue is a capable of inhibiting PCSK9 binding to human Low Density Lipoprotein Receptor (LDL-R).

In one embodiment the peptide analogues, compounds or PCSK9 inhibitors of the invention have an improved ability to bind PCSK9 compared to EGF(A) LDL-R(293-332) (SEQ ID 1).

As described above EGF(A) peptide analogues or compounds comprising such are considered PCSK9 inhibitors when such molecules have the ability to inhibit the binding of PCSK9 to LDL-R, by having and improved binding to PCSK9 compared to EGF(A) LDL-R(293-332) (SEQ ID 1).

In one embodiment the Kᵢ of the peptide analogues, compounds or PCSK9 inhibitors as described herein as measured in the PCSK9-LDL-R binding competitive ELISA assay (Assay I) is below 10 nM, such as below 8 nM or such as below 5 nM.

Functionality of EGF-(A) analogues and derivatives hereof may be further characterized by their ability to improve LDL uptake, such as described in WO2017/121850 Example D1.2. In one embodiment the peptide analogues, compounds or PCSK9 inhibitors of the invention increases LDL uptake in the presence of PCSK9. In one embodiment the peptide analogues, compounds or PCSK9 inhibitors of the invention are capable of reversing or reducing PCSK9 mediated reduction of LDL uptake.

In one embodiment the peptide analogues, compounds or PCSK9 inhibitors of the invention have a EC50 as measured in the LDL uptake assay of below 1500 nM, such as below 1000 nM or such as below 500 nM.

In an embodiment, a peptide analogue of the invention may be defined as comprising at least 1 amino acid substitution compared to SEQ ID NO: 1, and optionally an elongation. In an embodiment, a peptide analogue of the invention may be defined as comprising up to 15, up to 14, up to 13, up to 12, up to 11, up to 10, up to 9, up to 8, up to 7, up to 6, up to 5, up to 4, up to 3, up to 2 or 1 amino acid(s) substitution(s) compared to SEQ ID NO: 1, and optionally an elongation. This means that a peptide comprising an elongation in the N-terminal and/or in the C-terminal may comprise up to 15 amino acids substitutions in positions from 293 to 332 in addition to said elongation.

An amino acid “elongation” may also be referred to as “extension”. In an embodiment, peptide analogues of the invention comprise an elongation. Said elongation may be an addition of up to 50 amino acid residues in position N-terminal of SEQ ID NO: 1 or an analogue thereof, also referred to as an N-terminal elongation, meaning that a peptide of the invention may comprise up to 50 amino acids from position 292 down to, for example position 242. Additionally, or alternatively, said elongation may be an addition of up to 50 amino acid residues in position C-terminal of SEQ ID NO: 1 or analogue thereof, also referred to as a C-terminal elongation, meaning that a peptide of the invention may comprise up to 50 amino acids from position 333 up to, for example position 383.

Said elongation may be present either in N-terminal, in C-terminal or both. Said elongation may also be of any length between 0 and 50 amino acids on each side, independently of each other. In one embodiment, the peptide analogues of the invention comprise a N-terminal elongation of 1-50, 1-40, 10-40, 1-30, 10-30, 20-30, 20-40, 20-50, 30-50, 1-10, 11-20, 21-30, 31-40 or 41-50 amino acid residues or of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 amino acid residues. In addition or alternatively, the peptide analogues of the invention may comprise a C-terminal elongation of 1-50, 1-40, 10-40, 1-30, 10-30, 20-30, 20-40, 20-50, 30-50, 1-10, 11-20, 21-30, 31-40 or 41-50 amino acid residues or of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 amino acid residues.

An elongation may in some situation be referred to a substitution as a new amino acid residue is introduced, such as the 292A, 292Lys or 333Lys exemplified herein.

Minor truncations at the N-terminal and/or C-terminal of the EGF(A) peptide may be present in the EGF(A) peptide analogue.

In one embodiment the EGF(A) peptide comprise at least 35 amino acid residues, such as 36 amino acid residues, such as 37 amino acid residues, such as 38 amino acid residues or such as such as 39 amino acid residues. In one embodiment the EGF(A) peptide analogue according comprises an N-terminal truncation of 1-2amino acid residues. In one embodiment one or two N-terminal amino acid residues are deleted. In further embodiments the EGF(A) peptide analogue accordingly comprises an N-terminal truncation deleting at least or specifically amino acid 293Gly.

In further embodiments the EGF(A) peptide analogue comprises an N-terminal truncation deleting at least or specifically 293Gly-294Thr.

In one embodiment the EGF(A) peptide analogue comprises a C-terminal truncation of 1 amino acid residue. In one embodiment a single C-terminal amino acid residue is deleted. In on embodiment the peptide analogue comprises a C-terminal truncation deleting specifically amino acid 332Gly.

In addition, or alternatively, a peptide analogue of the invention may comprise at least one amino acid elongation in the N-terminal or the C-terminal for example in position 292 and/or 333.

The EGF(A) peptide analogue of the invention comprises the amino acid substitution of amino acid residue 301 from Asn to Leu, also described by Asn301Leu or simply 301Leu. In a specific embodiment, the EGF(A) peptide analogue comprises the substitution 301Leu.

In addition, or alternatively the EGF(A) peptide analogue comprises the amino acid residues 297Cys, 304Cys, 308Cys, 317Cys, 319Cys and 331Cys. Those Cys residues are wild type residues which may be engaged in disulphide bridges, such as the disulphide bridges between 297Cys and 308Cys, between 304Cys and 317Cys and between 319Cys and 331Cys.

In one embodiment, the EGF(A) peptide analogue comprises 301Leu and a number of further amino acid substitutions, as described above.

In one embodiment the EGF(A) peptide analogue comprises 301Leu, 310Asp and an amino acid substitution of 312Lys.

In one embodiment, the EGF(A) peptide analogue comprises 301Leu and 310Asp and wherein the peptide analogue does not have a substitution of 299Asp to Glu, Val or His.

In one embodiment the EGF(A) peptide analogue comprises 301Leu, 309Arg and 312Gly.

In one embodiment the EGF(A) peptide analogue comprises 301Leu and 309Arg with a proviso that the peptide analogue does not have a substitution of 310Asp to 310Lys or

In one embodiment the EGF(A) peptide analogue comprises 301Leu and 309Arg with a proviso that the peptide analogue does not have a substitution of 299Asp to Glu, Val or His.

In a further embodiment the peptide analogue does not have any of the substitutions

D310K, D310N, D310Q, D310Q, D31OR and D310A or even any substitution of 310Asp.

In one embodiment the EGF(A) peptide analogue comprises one, two, three or all four wild type residues: 295Asn, 296Gly, 298Leu and 302Gly.

In one embodiment the EGF(A) peptide analogue comprises one, two, three, four or all five wild type residues: 295Asn, 296Gly, 298Leu, 302Gly and 310Asp.

In one embodiment the peptide has 295Asn.

In one embodiment the peptide analogue has 296Gly. In one embodiment the peptide analogue has 298Leu. In one embodiment the peptide analogue has 302Gly. In one embodiment the peptide analogue has 310Asp.

In one embodiment the peptide analogue has two or more of 310Asp, 295Asn and 296Gly. In one embodiment the peptide analogue has all three of 310Asp, 295Asn and 296Gly.

The EGF(A) peptide analogue may comprise further amino acid substitutions as described herein. In one embodiment the analogue of the invention may further comprise one or more amino acid substitution in a position(s) selected from the group of positions: 293, 294, 296, 299, 300, 303, 305, 306, 309, 311, 312, 313, 314, 315, 316, 318, 320, 321, 322, 323, 324, 325, 326, 328, 329, 330 and 332.

In one embodiment the analogue of the invention may further comprise one or more amino acid substitution(s) in a position(s) selected from the group of positions: 293, 294, 299, 300, 303, 305, 306, 309, 311, 312, 313, 314, 316, 318, 321, 322, 323, 324, 325, 326, 328, 329, 330, 331 and 332.

In one embodiment the analogue of the invention may further comprise one or more amino acid substitution(s) in a position(s) selected from the 294, 299, 300, 303, 309, 312, 313, 314, 316, 318, 321, 322, 323, 324, 325, 326, 328, 329, 330 and 332.

In one embodiment the analogue of the invention may further comprise one or more amino acid substitution(s) in a position(s) selected from the 299, 300, 309, 313, 316, 318, 321, 322, 323, 324, 326, 328, 329, 330 and 332.

In one embodiment the analogue of the invention may further comprise one or further amino acid substitution(s) in a position(s) selected from the group of positions: 309, 312, 313, 321, 324, 328 and 332.

In a further embodiment the peptide analogue comprises either the wt amino acid residue or a different residue i.e. an amino acid substitution, in certain specific positions in addition to the amino acid residues specified herein above.

In one such embodiment the analogue of the invention comprises the amino acid residue Gly(G) or Asn(N) in position 293.

In one such embodiment the analogue of the invention comprises the amino acid residue Trp (W), Thr(T) or Gly(G) in position 294.

In one such embodiment the analogue of the invention comprises the amino acid residue Asp(D), Gly(G), Pro(P), Arg(R), Lys(K), Ser(S), Thr(T), Asn(N), Gln(Q), Ala(A), Ile(I), Leu(L), Met(M), Phe(F), Tyr(Y) or Trp(VV) in position 299.

In one such embodiment the analogue of the invention comprises the amino acid residue Asp(D), Gly(G), Pro (P), Arg(R), Lys(K), Ser(S), Thr(T), Asn(N), Gln(Q), Ala(A), Met(M), Phe(F), Tyr(Y) or Trp(W) in position 299.

In one such embodiment the analogue of the invention comprises the amino acid residue Asp(D), Ser (S), Arg(R), Leu (L), Ala (A), Lys(K) or Tyr(Y) in position 299.

In one such embodiment the analogue of the invention comprises the amino acid residue Asp(D) or Ala(A) in position 299.

In one such embodiment the analogue of the invention comprises the amino acid residue His(H) or Asn(N) in position 300.

In one such embodiment the analogue of the invention comprises the amino acid residue Val(V), Ser(S), Thr (T) or Ile (I) in position 307.

In one such embodiment the analogue of the invention comprises the amino acid residue Val(V) or Ile (I) in position 307.

In one such embodiment the analogue of the invention comprises Ser (S), Thr (T) or Ile (I) in position 307.

In one such embodiment the analogue of the invention comprises Ile (I) in position 307.

In one such embodiment the analogue of the invention comprises the amino acid residue Asn(N), Glu (E), His (H,) Arg (R), Ser (S) or Lys (K) in position 309.

In one such embodiment the analogue of the invention comprises the amino acid residue Asn(N), Arg (R), Ser (S) or Lys (K) in position 309.

In one such embodiment the analogue of the invention comprises the amino acid residue Asn(N) , Arg (R) or Ser (S) in position 309.

In one such embodiment the analogue of the invention comprises the amino acid residue Asn(N) or Arg (R) in position 309.

In one such embodiment the analogue of the invention comprises the amino acid residue Lys(K) or Arg (R) in position 309.

The EGF(A) peptide analogue may comprise several amino acid substitutions as described herein, such as one or more amino acid substitutions selected from the group of: 299Ala, 30711e and 321Glu.

In further embodiments, the EGF(A) peptide analogue comprises the amino acid residue Asp(D), Lys (K) or Glu(E) in position 321.

In further embodiments, the EGF(A) peptide analogue comprises the amino acid residue Asp(D) or Glu(E) in position 321.

In further embodiments, the EGF(A) peptide analogue comprises the amino acid residue Glu(E) in position 321.

In further embodiments, the EGF(A) peptide analogue comprises the amino acid residue GIn (Q) or Gly (G) in position 324.

In further embodiments, the EGF(A) peptide analogue comprises the amino acid residue Arg (R) or His (H) in position 329.

In further embodiments, the EGF(A) peptide analogue does not have a substitution of 300Asn(N) to Pro(P).

The EGF(A) domain of LDL-R includes a Lysine in position 312 which may be useful for substitution as described herein. In embodiments where attachment of the substituent to 312 is not wanted 312Lys may be substituted by another amino acid as described herein.

In one embodiment, Lys in position 312 is substituted by an amino acid residue selected from: Gly, Pro, Asp, Glu, Arg, His, Ser, Thr, Asn, GIn, Ala, Val, Ile, Leu, Met, Phe and Tyr. In one embodiment, Lys in position 312 is substituted by an amino acid residue selected from: Gly, Asp, Glu, Ser, Thr, Asn, Ala, Val, Ile, Leu, Phe and Tyr. In one embodiment, Lys in position 312 is substituted by an amino acid residue selected from: Asp, Glu, Thr, Asn, Ile, Leu, Phe and Tyr. In one embodiment, 312Lys is substituted by 312Asp, 312Glu, 312Thr, 312Asn, 312Ile or 312Phe. In one embodiment, 312Lys is substituted by 312Glu, 312Asp, 312GIn or 312Arg.

In one embodiment, 312Lys is substituted by 312Glu, 312Thr, 312Asn, 312Ile, 312Phe or 312Tyr. In one embodiment, 312Lys is substituted by 312Glu, 312Asn or 312Ile,

In one embodiment, 312Lys is substituted by 312Glu or 312Arg. In one embodiment 312Lys is substituted by 312Arg. In one embodiment, 312Lys is substituted by 312Glu.

To include an option for attaching the substituent in various positions (see further below), a Lys may be introduced by amino acid substitution of a wild type residue of SEQ ID NO.: 1 or by a peptide elongation of SEQ ID NO.: 1, such as a 292Lys or a 333Lys.

In cases where more than one substituent is desired one may be via 312Lys while the second is via a Lys introduced by peptide elongation or substitution in SEQ ID NO.: 1.

In one embodiment the peptide analogue of SEQ ID NO: 1 comprises at least one Lys residue in a position selected from the group of: 292Lys, 293Lys, 294Lys, 296Lys, 299Lys, 300Lys, 303Lys, 305Lys, 306Lys, 309Lys, 311Lys, 312Lys, 313Lys, 314Lys, 315Lys, 316Lys, 318Lys, 320Lys, 321Lys, 322Lys, 323Lys, 324Lys, 325Lys, 326Lys, 327Lys, 328Lys, 329Lys, 330Lys, 332Lys and 333Lys.

In one embodiment the peptide analogue of SEQ ID NO: 1 comprises at least one Lys residue in a position selected from the group of: 292Lys, 293Lys, 294Lys, 299Lys, 300Lys, 303Lys, 305Lys, 306Lys, 309Lys, 311Lys, 312Lys, 313Lys, 314Lys, 315Lys, 316Lys, 318Lys, 320Lys, 321Lys, 322Lys, 323Lys, 324Lys, 325Lys, 326Lys, 327Lys, 328Lys, 329Lys, 330Lys, 332Lys and 333Lys.

In one embodiment the peptide analogue of SEQ ID NO: 1 comprises at least one Lys residue in a position selected from the group of: 292Lys, 293Lys, 294Lys, 300Lys, 303Lys, 305Lys, 306Lys, 309Lys, 311Lys, 312Lys, 313Lys, 314Lys, 316Lys, 318Lys, 321Lys, 322Lys, 323Lys, 324Lys, 325Lys, 326Lys, 327Lys, 328Lys, 329Lys, 330Lys, 332Lys and 333Lys.

In one embodiment the peptide analogue of SEQ ID NO: 1 comprises at least one Lys residue in a position selected from the group of: 292Lys, 293Lys, 294Lys, 300Lys, 303Lys, 305Lys, 306Lys, 311Lys, 312Lys, 313Lys, 314Lys, 316Lys, 318Lys, 322Lys, 323Lys, 324Lys, 325Lys, 326Lys, 327Lys, 328Lys, 329Lys, 330Lys, 332Lys and 333Lys.

In one embodiment the peptide analogue of SEQ ID NO: 1 comprises at least one Lys residue in a position selected from the group of: 292Lys, 293Lys, 294Lys, 300Lys, 303Lys, 305Lys, 306Lys, 311Lys, 313Lys, 314Lys, 316Lys, 318Lys, 322Lys, 323Lys, 324Lys, 325Lys, 326Lys, 327Lys, 328Lys, 329Lys, 330Lys, 332Lys and 333Lys.

In addition or alternatively, the peptide analogue of the invention comprises at least one amino acid substitution selected from 292Lys, 293Lys, 294Lys, 295Lys, 296Lys, 298Lys, 299Lys, 301Lys, 302Lys, 303Lys, 305Lys, 306Lys, 307Lys, 309Lys, 310Lys, 311Lys, 313Lys, 314Lys, 315Lys, 316Lys, 318Lys, 320Lys, 321Lys, 322Lys, 323Lys, 324Lys, 325Lys, 326Lys, 327Lys, 328Lys, 329Lys, 330Lys, 332Lys and 333Lys.

In a further embodiment, the EGF(A) peptide analogue of the invention comprises at least one amino acid substitution selected from:292Lys, 293Lys, 294Lys, 295Lys, 296Lys, 298Lys, 299Lys, 302Lys, 303Lys, 305Lys, 306Lys, 307Lys, 309Lys, 311Lys, 313Lys, 314Lys, 315Lys, 316Lys, 318Lys, 320Lys, 321Lys, 322Lys, 323Lys, 324Lys, 325Lys, 326Lys, 327Lys, 328Lys, 329Lys, 330Lys, 332Lys and 333Lys.

In a further embodiment, the EGF(A) peptide analogue of the invention comprises at least one amino acid substitution selected from 292Lys, 293Lys, 294Lys, 295Lys, 296Lys, 298Lys, 299Lys, 303Lys, 305Lys, 306Lys, 309Lys, 311Lys, 313Lys, 314Lys, 315Lys, 316Lys, 318Lys, 320Lys, 321Lys, 322Lys, 323Lys, 324Lys, 325Lys, 326Lys, 327Lys, 328Lys, 329Lys, 330Lys, 332Lys and 333Lys.

In a further embodiment, the EGF(A) peptide analogue of the invention comprises at least one amino acid substitution selected from 292Lys, 293Lys, 294Lys, 295Lys, 296Lys, 299Lys, 303Lys, 305Lys, 306Lys, 309Lys, 311Lys, 313Lys, 314Lys, 315Lys, 316Lys, 318Lys, 320Lys, 321Lys, 322Lys, 323Lys, 324Lys, 325Lys, 326Lys, 327Lys, 328Lys, 329Lys, 330Lys, 332Lys and 333Lys.

In a further embodiment, the EGF(A) analogue peptide of the invention comprises at least one amino acid substitution selected from 292Lys, 293Lys, 294Lys, 296Lys, 299Lys, 303Lys, 305Lys, 306Lys, 309Lys, 311Lys, 313Lys, 314Lys, 315Lys, 316Lys, 318Lys, 320Lys, 321Lys, 322Lys, 323Lys, 324Lys, 325Lys, 326Lys, 327Lys, 328Lys, 329Lys, 330Lys, 332Lys and 333Lys.

In a further embodiment, the EGF(A) peptide analogue of the invention comprises at least one amino acid substitution selected from 292Lys, 293Lys, 294Lys, 299Lys, 303Lys, 305Lys, 306Lys, 309Lys, 311Lys, 313Lys, 314Lys, 315Lys, 316Lys, 318Lys, 320Lys, 321Lys, 322Lys, 323Lys, 324Lys, 325Lys, 326Lys, 327Lys, 328Lys, 329Lys, 330Lys, 332Lys and 333Lys.

In a further embodiment, the EGF(A) peptide analogue of the invention comprises at least one amino acid substitution selected from 292Lys, 293Lys, 294Lys, 299Lys, 303Lys, 305Lys, 306Lys, 309Lys, 311Lys, 313Lys, 314Lys, 315Lys, 316Lys, 318Lys, 320Lys, 321Lys, 322Lys, 323Lys, 324Lys, 325Lys, 326Lys, 327Lys, 328Lys, 329Lys, 330Lys, 332Lys

In a further embodiment, the EGF(A) peptide analogue of the invention comprises at least one amino acid substitution selected from 292Lys, 293Lys, 294Lys, 299Lys, 303Lys, 305Lys, 306Lys, 310Lys, 311Lys, 313Lys, 314Lys, 315Lys, 316Lys, 318Lys, 320Lys, 321Lys, 322Lys, 323Lys, 324Lys, 325Lys, 326Lys, 327Lys, 328Lys, 329Lys, 330Lys, 332Lys and 333Lys.

In a further embodiment, the EGF(A) peptide analogue of the invention comprises at least one amino acid substitution selected from 292Lys, 293Lys, 294Lys, 299Lys, 303Lys, 305Lys, 306Lys, 309Lys, 310Lys, 311Lys, 313Lys, 314Lys, 315Lys, 316Lys, 318Lys, 321Lys, 322Lys, 323Lys, 324Lys, 325Lys, 326Lys, 327Lys, 328Lys, 329Lys, 330Lys, 332Lys and 333Lys.

In a further embodiment, the EGF(A) peptide analogue of the invention comprises at least one amino acid substitution selected from 292Lys, 293Lys, 294Lys, 303Lys, 305Lys, 306Lys, 310Lys, 311Lys, 313Lys, 314Lys, 315Lys, 316Lys, 318Lys, 321Lys, 322Lys, 323Lys, 324Lys, 325Lys, 326Lys, 327Lys, 328Lys, 329Lys, 330Lys, 332Lys and 333Lys. In one embodiment, the peptide analogues of the invention do not comprise any of the following substitutions: 296K, 298K, 301K, 302K and 307K.

In one embodiment, the peptide analogues of the invention do not comprise any of the following substitution: 296K, 298K, 301K, 302K, 307K and 310K.

In one embodiment, the peptide analogues of the invention do not comprise any of the following substitution: 296K, 298K, 301K, 302K, 307, and 295K.

In one embodiment, the peptide analogues of the invention do not comprise any of the following substitution: 296K, 298K, 301K, 302K, 307K and 295D.

In a particular embodiment, the peptide analogue of the invention comprises 1 or 2, of such Lys substitutions.

In addition, or alternatively, the peptide of the invention may comprise 312Lys.

In one embodiment the peptide analogue of the invention comprises two Lys residues. In one embodiment the peptide analogue of the invention comprises two Lys residues selected from the pairs consisting of:

| | | | |
|---|---|---|---|
| i. | 293K and 294K | xiv. | 313K and 321K |
| ii. | 293K and 312K | xv. | 313K and 324K |
| iii. | 293K and 333K | xvi. | 313K and 328K |
| iv. | 309K and 313K | xvii. | 313K and 332K |
| v. | 309K and 324K | xviii. | 313K and 333K |
| vi. | 309K and 328K | xix. | 314K and 333K |
| vii. | 309K and 332K | xx. | 321K and 332K |
| viii. | 309K and 333K | xxi. | 321Kand 333K |
| ix. | 311Kand 313K | xxii. | 324K and 333K |
| x. | 312K and 333K | xxiii. | 324K and 328K |
| xi. | 312K and 313K | xxiv. | 328K and 333K |
| xii. | 312K and 314K | xxv. | 330K and 333K and |
| xiii. | 313K and 314K | xxvi. | 332K and 333K. |

As seen herein above various peptide analogues are provided by the present invention. In a further embodiment the EGF(A) peptide analogue according to the invention comprises at least two amino acid substitutions identified by any of the groups i-xxiv shown below compared to SEQ ID NO.:1.

In a still further embodiment, the EGF(A) peptide analogue of the invention consists of the amino acid substitutions identified by any of the groups i-xxiv as shown below.

In a further embodiment the EGF(A) peptide analogue according to the invention comprises at least two amino acid substitutions identified by any of the groups i-xvi shown below compared to SEQ ID NO.:1.

In a still further embodiment, the EGF(A) peptide analogue of the invention consists of the amino acid substitutions identified by any of the groups i-xvi as shown below.
i. 301Leu and 309Arg
ii. 301Leu, 309Arg, 312Glu
iii. 301Leu, 30711e and 309Arg
iv. 301Leu, 30711e, 309Arg and 312Glu
v. 301Leu, 309Arg and 321Glu
vi. 301Leu, 309Arg, 321Glu and 312Glu
vii. 301Leu, 307Ile, 309Arg and 299Ala
viii. 301Leu, 307Ile, 309Arg, 299Ala and 312Glu
ix. 301Leu and 309Arg and at least one Lys substitution
x. 301Leu, 309Arg, 312Glu and at least one Lys substitution
xi. 301Leu, 307Ile and 309Arg and at least one Lys substitution
xii. 301Leu, 307Ile, 309Arg and 312Glu and at least one Lys substitution
xiii. 301Leu, 309Arg and 321Glu and at least one Lys substitution
xiv. 301Leu, 309Arg, 321Glu and 312Glu and at least one Lys substitution
xv. 301Leu, 307Ile, 309Arg and 299Ala and at least one Lys substitution or
xvi. 301Leu, 307Ile, 309Arg, 299Ala and 312Glu and at least one Lys substitution.

In a further embodiment the EGF(A) peptide analogue according to the invention comprises at least two amino acid substitutions identified by any of the groups xvii-xx shown below compared to SEQ ID NO.: 1.

In a still further embodiment, the EGF(A) peptide analogue of the invention consists of at the amino acid substitutions identified by any of the groups xvii-xx as shown below.
xvii. 301Leu and 309Lys
xviii. 301Leu, 309Lys and 312Glu
xix. 301Leu and 309Lys and at least one further Lys substitution
xx. 301Leu, 309Lys and 312Glu and at least one further Lys substitution.

In a further embodiment the EGF(A) peptide analogue according to the invention comprises at least two amino acid substitutions identified by any of the groups xxi-xxiv shown below compared to SEQ ID NO.: 1.

In a still further embodiment, the EGF(A) peptide analogue of the invention consists of the amino acid substitution identified by any of the groups xxi-xxiv as shown below
xxi. 301Leu and 307Ile,
xxii. 301Leu, 307Ile and 312Glu
xxiii. 301Leu and 307Ile and at least one further Lys substitution and
xxiv. 301Leu, 3307Ile and 312Glu and at least one further Lys substitution.

In further specific embodiments the peptide analogue or the peptide analogue of the compounds according to the invention comprises or consists of anyone of the amino acid sequences identified by SEQ ID 1 to 114.

In one embodiment the peptide analogue comprises or consists of anyone of the amino acid sequences identified by SEQ ID NO.: 2-114.

In one embodiment the peptide analogue comprises or consists of anyone of the amino acid sequences identified by SEQ ID NO.: 2-47 and 49-114.

In one embodiment the peptide analogue comprises or consists of anyone of the amino acid sequences identified by anyone of the amino acid sequences SEQ ID NO.: 2-44, 46, 47 and 49-1-114.

In one embodiment the peptide analogue comprises or consists of anyone of the amino acid sequences identified by of SEQ ID NO.: 2-44, 46, 47, 49-53, 55, 58-114.

In one embodiment the peptide analogue comprises or consists of anyone of the amino acid sequences identified by SEQ ID NO.: 2-4, 6-44, 46, 47, 49-53, 55, 58-114.

In one embodiment the peptide analogue comprises or consists of anyone of the amino acid sequences identified by SEQ ID NO.: 2-4, 6-19, 21-44, 46, 47, 49-53, 55, 58-114.

In one embodiment the peptide analogue comprises or consists of anyone of the amino acid sequences identified by SEQ ID NO.: 2-4, 6-19, 21-44, 46, 47, 49-53, 55, 58-114.

In one embodiment the peptide analogue comprises or consists of anyone of the amino acid sequences identified by SEQ ID NO.: 3, 6 and 81.

In one embodiment the peptide analogue comprises or consists of anyone of the amino acid sequences identified by SEQ ID NO.: 4, 8, 11, 15-19, 21, 22, 24, 31-42, 44, 51-53, 70-73, 77-78, 91, 94, 95, 97-102, 104-109, 112-114.

In one embodiment the peptide analogue comprises or consists of anyone of the amino acid sequences identified by SEQ ID NO.: 4, 6, 32,72, 76, 78, 98, 104 and 105.

### Intermediate Compounds

The present invention also relates to peptide analogues which may be incorporated in the derivatives of the invention. Such peptide analogues may be referred to as “intermediate product” or “intermediate compound”. They are in the form of novel LDL-R(293-332) analogues, which as described above can be incorporated in EGF(A) derivatives of the invention as further describe below. Such peptide analogues are as defined in the above section.

In particular, a peptide analogue, or intermediate peptide, according to the present invention may be referred to as a peptide analogue of sequence SEQ ID NO: 1.

In one aspect the invention relates to a EGF(A) peptide analogue as described herein for use in the manufacture of a EGF(A) compound, such as a EGF(A) derivative. Other features, definitions, aspects and embodiments disclosed herein in connection with peptide analogues of the invention may also be applicable to the intermediate products of the invention.

### EGF(A) Derivatives

The peptides analogues of the invention may further comprise a substituent and thereby become derivative compounds.

The term “derivative” generally refers to a compound which may be prepared from a native peptide or an analogue thereof by chemical modification, in particular by covalent attachment of one or two substituents.

The terms “derivative of the invention”, “EGF(A) derivative”, “EGF(A) derivative or “LDL-R(293-332) derivative” or “derivative of a LDL-R(293-332) analogue” as used herein refers to as a peptide to which one or two substituents are attached. Each of these may, also or alternatively, be referred to as a side chain. In other words, a “derivative of the invention” comprises a peptide i.e. a peptide sequence, which herein is an EGF(A) peptide analogue, and at least one, including such as one or two, substituent(s).

The terms “substituent” is used to describe a moiety covalently bond to the EGF(A) peptide e.g. the substituent is a moiety not part of the EGF(A) peptide itself.

In one embodiment the one or more substituent(s) is/are attached to a nitrogen atom of the EGF(A) peptide analogue. In one embodiment the one or more substituent(s) is/are attached to an amino group of the EGF(A) peptide analogue. In one embodiment the one or more substituent(s) is/are attached to the N-terminal amino acid of the EGF(A) peptide analogue or to a Lys residue of the EGF(A) peptide analogue. In one embodiment the one or more substituent(s) is/are attached to the N-terminal amino acid of the EGF(A) peptide analogue. In one embodiment the one or more substituent(s) is/are attached to the alpha-nitrogen of the N-terminal amino acid residue of the EGF(A) peptide analogue. In one embodiment the one or more substituent(s) is/are attached to a Lys residue in the EGF(A) peptide analogue. In one embodiment the one or more substituent(s) is/are attached to the epsilon-nitrogen of a Lys residue in the EGF(A) peptide analogue.

Examples of substituents are various and further described below.

In one aspect, the invention relates to an EGF(A) derivative comprising an EGF(A) peptide analogue and at least one substituent. In one embodiment the substituent of the derivative comprises at least one fatty acid group. For all embodiments the term EGF(A) derivative also encompasses any pharmaceutically acceptable salt, amide, or ester thereof.

In one embodiment the EGF(A) derivative is a TFA salt, an ammonium salt, a sodium, a acetate salt or a chloride salt. In one embodiment the EGF(A) derivative is an ammonium salt or a sodium salt.

### Substituents

A substituent is a moiety attached to an EGF(A) peptide analogue. According to the invention it is preferred that the moiety e.g. the substituent has no or minimal effect on the functionality of the EGF(A) peptide while adding other beneficial properties, such as longer half-life and/or improved exposure after oral dosing.

It follows that the derivatives, as well as the analogues of the invention described above, have the ability to bind to PCSK9. Such binding to PCSK9 inhibits PCSK9 binding to the LDL-R, thereby preventing LDL-R degradation hence increasing the clearance of LDL-C and atherogenic lipoproteins.

In a specific embodiment, the derivatives and analogues of the invention have an improved ability to bind to PCSK9, for example compared to native LDL-R(293-332) or to other PCSK9-binding compounds. The analogues and derivatives of the invention can for example be tested for their ability to inhibit PCSK9 binding to LDL-R using the assay described in Assay I herein.

In an embodiment the substituent is aimed at improving the functionality of the peptides.

In one embodiment the substituent increase half-life of the peptide analogue in a way that the plasma half-live of a derivative comprising a backbone peptide and a substituent have an increase half-life compared to the half-life of the backbone. Methods for determining half-life in different species are well known in the art and exemplified in WO2017/121850 for mice and dogs (Section D2 and D5).

In one embodiment the EGF(A) derivative according to the invention has a half-life above 4 hours.

In one embodiment the EGF(A) derivative according to the invention has a half-life above 6 hours, such as above 8 hours or such as above 10 hours in mice measured after either subcutaneously or intravenously dosing.

In one embodiment the EGF(A) derivative according to the invention has a half-life above 25 hours in dogs.

In one embodiment the EGF(A) derivative according to the invention has a half-life above 50 hours, such as above 100 hours or such as above 150 hours in dogs.

In one embodiment, a half-life extending substituent is a protein moiety. In a further such embodiment the protein moiety may include human albumin, an Fc-domain or an unstructured protein extension. In a further embodiment the protein moiety may by fused to the peptide analogue. In a further embodiment, the protein moiety is Fc domain and the Fc domain is fused to the peptide analogue. When an Fc fusion is prepared the resulting compound will usually be divalent as two Fc-polypeptides will form one Fc-domain.

In one embodiment the substituent is not a protein moiety. In one embodiment the substituent is not a protein moiety fused to the EGF(A) peptide analogue. In one embodiment the protein moiety is not an Fc domain.

In another embodiment the substituent is a non-protein moiety.

In a particular embodiment, the substituent is capable of forming non-covalent complexes with albumin, thereby promoting the circulation of the derivative within the blood stream, and also having the effect of protracting the time of action of the derivative. In a particular embodiment, the substituent is capable of protracting the time of action of the EGF(A) compound without substantially decreasing its binding capacity to PCSK9.

In one embodiment the EGF(A) derivative comprises a half-life extending substituent. Various half-life extending substituents are well-known in the art and include in particular albumin binders comprising a fatty acid group as described further below, and such albumin binders are non-protein substituents.

The substituent comprises at least one fatty acid group.

In a particular embodiment, the fatty acid group comprises a carbon chain which contains at least 8 consecutive —CH₂- groups. In one embodiment the fatty acid group comprise at least 10 consecutive —CH₂- groups, such as least 12 consecutive —CH₂- groups, at least 14 consecutive —CH₂- groups, at least 16 consecutive —CH₂- groups, at least 18 consecutive —CH₂- groups.

In one embodiment the fatty acid group comprises 8-20 consecutive —CH₂- groups.

In one embodiment the fatty acid group comprises 10-18 consecutive —CH₂- groups. In one embodiment the fatty acid group comprises 12-18 consecutive —CH₂- groups. In one embodiment the fatty acid group comprises 14-18 consecutive —CH₂- groups.

In situations where the derivative comprise two substituents, an increased half-life may be obtained with shorter fatty acid groups, thus in an embodiment where the derivate comprise two substituents the fatty acid groups may comprise at least 8 consecutive —CH₂- groups, such as least 10 consecutive —CH₂- groups, such as least 12 consecutive —CH₂- groups, at least 14 consecutive —CH₂- groups, at least 16 consecutive —CH₂- groups.

In a further embodiment where the derivative comprises two substituents, the substituents each comprise a fatty acid group comprising 8-18 consecutive —CH₂- groups. In further such embodiments the fatty acid groups comprise 10-18 consecutive —CH₂- groups, such as 12-18 consecutive —CH₂- groups, such as 14-18 consecutive —CH₂- groups.

The term “fatty acid group” as used herein may be referred to as chemical group comprising at least one functional group being a Brønsted-Lowry acid with a pKa <7. Non-limiting examples of such functional groups that are Brønsted-Lowry acids include a carboxylic acid (including also carboxyphenoxy), a sulphonic acid, a tetrazole moiety.

In one embodiment said fatty acid group comprises a functional group selected from a carboxylic acid, a sulphonic acid, a tetrazole moiety, a methylsulfonylcarbamoylamino (MSU) moiety and a 3-Hydroxy-isoxazolelsoxazole moiety. Accordingly, the half-life extending substituent of the invention in an embodiment comprises a carboxylic acid, a sulphonic acid, a tetrazole moiety, a methylsulfonylcarbamoylamino moiety or a hydroxy-isoxazolelsoxazole moiety further including 8-20 consecutive —CH₂- groups as defined by:

Chem. 1: HOOC—(CH₂)ₙ—CO-* wherein n is an integer in the range of 8-20, which may also be referred to as a C(n+2) diacid or as

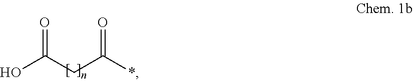

wherein n is an integer in the range of 8-20,

Chem. 2: 5-tetrazolyl-(CH₂)ₙ—CO-* wherein n is an integer in the range of 8-20, which may also be referred to as

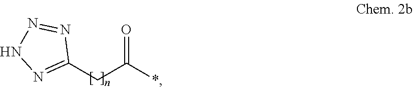

wherein n is an integer in the range of 8-20.

Chem. 3: HOOC—(C₆H₄)—O—(CH₂)ₘ—CO-* wherein n is an integer in the range of 8-20, which may also be referred to as

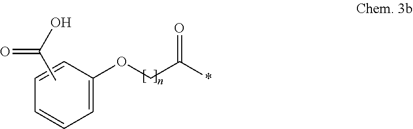

wherein the carboxy group is in position 2, 3 or 4 of the (C₆H₄) group of Chem. 3 and wherein m is an integer in the range of 8-11

Chem. 4: HO—S(O)₂—(CH₂)ₙ—CO-* wherein n is an integer in the range of 8-20, which may also be referred to as

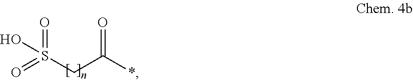

wherein n is an integer in the range of 8-20,

Chem. 5: MeS(O)₂NH(CO)NH—(CH₂)ₙ—CO-* wherein n is an integer in the range of 8-20, which may also be referred to as.

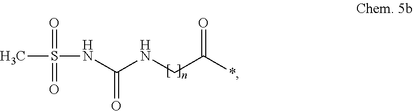

wherein n is an integer in the range of 8-20,

Chem. 6: 3-HO-Isoxazole-(CH₂)ₙ—CO-* wherein n is an integer in the range of 8-20, which may also be referred to as

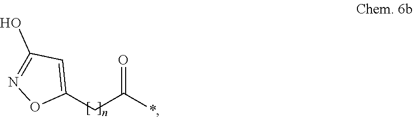

wherein n is an integer in the range of 8-20.

The term functional group in its acidic form is referred to as FG-H and its form as conjugated base referred to as FG⁻. The term “functional group with a pKa <7” as used herein may be referred to as a Brønsted-Lowry acid which in the form of its methyl derivative (CH₃-FG-H) in aqueous solution has a equilibrium pKa of below 7, wherein the pKa is the −log to the equilibrium constant (Ka) of the equilibrium shown below:

CH₃—FG—H+H₂O [symbol] CH₃-FG⁻+H₃O⁺.

Methods for the determination of pKa are well known in the art. Such a method has for example been described by Reijenga et al. in Anal Chem Insights 2013 (2013; 8: 53-71).

Substituents according to the invention in an embodiment comprise one or more linker elements. The linker elements may be linked to the fatty acid group by amide bonds and referred to as Z₂-Z₁₀. As further defined herein below the number of linker elements may be at most 10.

In a specific embodiment, the substituent is of Formula I:

Z₁-Z₂-Z₃-Z₄-Z₅-Z₆-Z₇-Z₈-Z₉-Z₁₀ [I]

wherein

Z₁ is selected from:

Chem. 1: HOOC—(CH₂)ₙ—CO-* or

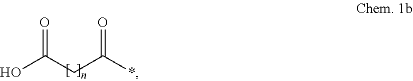

Chem. 2: 5-tetrazolyl-(CH₂)ₙ—CO-* or

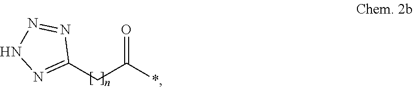

Chem. 3: HOOC—(C₆H₄)—O—(CH₂)ₘ—CO-* or

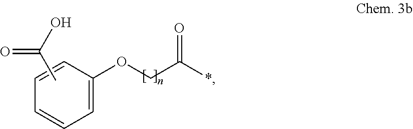

wherein the carboxy group is in position 2, 3 or 4 of —(C₆H₄)-,

Chem. 4: HOS(O)₂—(CH₂)ₙ—CO-* or

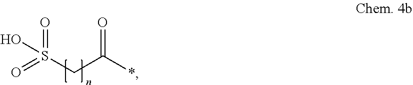

Chem. 5: MeS(O)₂NH₂N(CO)NHN—(CH₂)ₙ—CO-* or

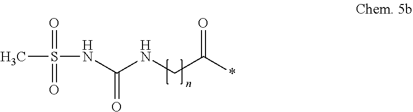

and

Chem. 6: 3-HO-Isoxazole-(CH₂)ₙ—CO-* or

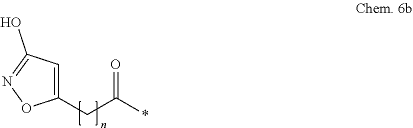

wherein n is an integer in the range of 8-20 and m is an integer in the range of 8-11.

In a particular embodiment, n is 8, 9, 10, 11, 12, 13, 14, 15, 16, 17, 18, 19 or 20 in Chem. 1 or 1b. In a particular embodiment, n is 8, 9, 10, 11, 12, 13, 14, 15, 16, 17, 18, 19 or 20 in Chem. 2 or 2b. In a particular embodiment, n is 8, 9, 10, 11, 12, 13, 14, 15, 16, 17, 18, 19 or 20 in Chem. 4 or 4b. In a particular embodiment, m is 8, 9, 10 or 11 in Chem. 3 or 3b. In a particular embodiment, n is 8, 9, 10, 11, 12, 13, 14, 15, 16, 17, 18, 19 or 20 in Chem. 5 or 5b.

In a particular embodiment, n is 8, 9, 10, 11, 12, 13, 14, 15, 16, 17, 18, 19 or 20 in Chem. 6 or 6b.

In a particular embodiment, the symbol * indicates the attachment point to the nitrogen in Z₂. In another embodiment, where Z₂ is a bond, the symbol * indicates the attachment point to the nitrogen of the neighbouring Z element.

The term “bond” as used in the context of Formula I means a covalent bond. When a component of Formula I (Z₁-Z₁₀) is defined as a bond, it is equivalent to a formula I wherein said component is absent.

The indication herein below that any of Z₂-Z₁₀ is a bond may also be read as any of Z₂-Z₁₀ being absent. Logically “a bond” cannot follow “a bond”. The indication “a bond” here thus means that the previous Z element is covalently linked to the next Z element that is not “a bond” (or absent).

The linker elements Z₂-Z₁₀ are selected from chemical moieties that are capable of forming amide bounds, including amino acid like moieties, such as Glu, γGlu (also termed gammal Glu or gGlu and defined by *—NH—CH—(COON)—CH₂—CH₂—CO-*), Gly, Ser, Ala, Thr, Ado, Aeep, Aeeep and TtdSuc and further moieties defined below.

Z₂ is selected from

Chem. 7: *—NH—SO₂—(CH₂)₃—CO-* or

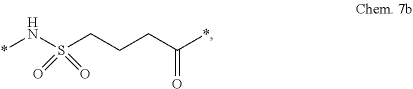

Chem. 8: *—NH—CH₂—(C₆H₁₀)—CO-* or

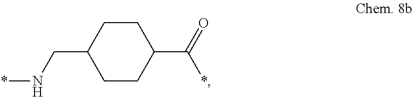

and a bond.

Z₃ is selected from γGlu, Glu, or a bond.

Z₃ is selected from γGlu, Glu, or a bond when Z₂ is Chem. 7 or Chem. 7b.

Z₃ is selected from γGlu, Glu, or a bond, provided that Z₃ is selected from γGlu, Glu when Z₂ is Chem. 8.

Z₃ is selected from γGlu and Glu when Z₂ is Chem. 8.

Z₄, Z₅, Z₆, Z₇, Z₈, Z₉ are selected, independently of each other, from Glu, γGlu, Gly, Ser, Ala, Thr, Ado, Aeep, Aeeep, TtdSuc and a bond.

Glu, Gly, Ser, Ala, Thr are amino acid residues as well known in the art.

γGlu is of formula Chem. 9: *—NH—CH(COOH)—(CH₂)₂—CO-* which is the same as

Chem. 9b:

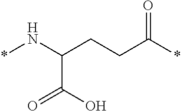 [SMILES: *NC(CCC(*)=O)C(=O)O]

and may also be referred to as gGlu.

TtdSuc is of formula Chem. 10:

*—NH—(CH₂)₃—O—(CH₂)₂—O—(CH₂)₂O—(CH₂)₃—NHCO* or

*—NH—CH₂CH₂CH₂OCH₂CH₂OCH₂CH₂OCH₂CH₂CH₂NHCO* which is the same as

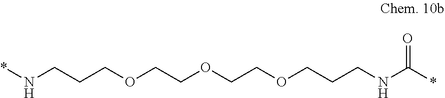 [SMILES: *NCCCOCCOCCOCCCNC(*)=O]

Ado is of formula Chem. 11: *—NH—(CH₂)₂—O—(CH₂)₂—O—CH₂—CO-* may also be referred to as 8-amino-3,6-dioxaoctanoic acid and which is the same as

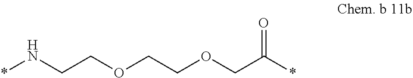 [SMILES: *NCCOCCOCC(*)=O]

Aeep is of formula Chem. 12: *NH—CH₂CH₂OCH₂CH₂OCH₂CH₂CO*, which may also be referred to as

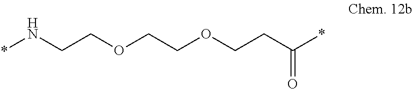 [SMILES: *NCCOCCOCCC(*)=O]

Aeeep is of formula Chem. 13: *NH—CH₂CH₂OCH₂CH₂OCH₂CH₂OCH₂CH₂CO*, which may also be referred to as

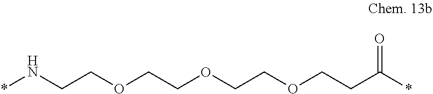 [SMILES: *NCCOCCOCCOCCC(*)=O]

Z₁₀ is selected from a bond, and Chem. 14: *—NH—CH₂—(C₆H₄)—CH₂-*, which may also be referred to as

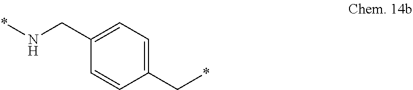 [SMILES: *Cc1ccc(CN*)cc1]

In a particular embodiment, when Z₁₀ is Chem. 14, the substituent is attached to the N-terminal amino group of said peptide.

In another embodiment, when Z₁₀ is a bond, said substituent is attached to the epsilon position of a Lys residue present in said peptide or to the N-terminal amino acid residue of said peptide.

In one embodiment the derivative comprises two substituents. In one such embodiment the two substituents are identical. In one such embodiment the two substituents are different. In one embodiment the two substituents are attached to nitrogen atoms of the EGF(A) peptide analogue. In one embodiment the two substituents are attached to amino groups of the EGF(A) peptide analogue. In one embodiment the two substituents are attached to the N-terminal amino acid EGF(A) and to a Lys residue of the EGF(A) peptide analogue. In one embodiment, one substituent is attached the alpha-nitrogen of the N-terminal amino acid residue of the EGF(A) peptide analogue and one substituent is attached to a Lys residue of the EGF(A) peptide analogue. In one embodiment two substituents are attached to the N-terminal amino acid of the EGF(A) peptide analogue. In one embodiment the two substituents are attached to different Lys residues of the EGF(A) peptide analogue. In one embodiment the two substituents are attached to the epsilon-nitrogens of different Lys residues in the EGF(A) peptide analogue.

In one embodiment where two substituents are present, Z₁₀ is Chem. 14 in one substituent which is attached to the N-terminal amino group of a peptide analogue and Z₁₀ is a bond in the other substituent which is attached to the epsilon position of a Lys residue present in said peptide analogue.

In another embodiment where two substituents are present, Z₁₀ is a bond in one substituent which is attached to the N-terminal amino group of a peptide analogue and Z₁₀ is a bond in the other substituent which is attached to the epsilon position of a Lys residue present in said peptide analogue.

In another embodiment where two substituents are present, Z₁₀ is a bond in both substituents and each of the two substituents is attached to the epsilon position of different Lys residues present in a peptide analogue.

In a particular embodiment, the derivatives of the invention may be prepared from an EGF(A) peptide analogue by covalent attachment of one or two substituent(s).

In a particular embodiment, the two substituents are of Formula I:

Z₁-Z₂-Z₃-Z₄-Z₅-Z₆-Z₇-Z₈-Z₉-Z₁₀ [I].

Z₁ to Z₁₀ are as defined above. In a particular embodiment, the two substituents are of formula I and are identical, meaning that selected Z₁ to Z₁₀ are the same in both substituents. In another embodiment, the two substituents are of formula I and are different, meaning that one or more of selected Z₁ to Z₁₀ are different between one substituent and the other.

Specific substituents

As seen above various substituents can be prepared by the persons skilled in the art. The substituents include in the present application are thus not to be considered limiting to the invention.

In one embodiment the one or two substituent(s) is/are selected from the group of substituents consisting of:

HOOC—(CH₂)₁₈—CO-gGlu-2xADO
HOOC—(CH2)₁₈—CO—NH—CH2—(C₆Hio)—CO-gGlu-2xADO
HOOC—(CH₂)₁₆—CO-gGlu-2xADO
HOOC—(CH₂)₁₆—CO-gGlu-2xADO—NH—CH₂—(C₆H₄)—CH₂
HOOC—(CH₂)₁₆—CO-gGlu
HOOC—(CH₂)₁₆—CO—NH—CH₂—(C₆H₁₀)—CO-gGlu-2xADO
HOOC—(CH₂)₁₄—CO-gGlu-2xADO
HOOC—(CH₂)₁₄—CO-gGlu-
HOOC—(CH₂)₁₄—CO-gGlu-2xADO—
HOOC—(CH₂)₁₂—CO-gGlu-2xADO
4-HOOC—(C₆H₄)—O—(CH₂)₁₀—CO-gGlu-2xADO
4-HOOC—(C₆H₄)—O—(CH₂)₁₀—CO-gGlu-3xADO
4-HOOC—(C₆H₄)—O—(CH₂)₁₀—CO-gGlu
4-HOOC—(C₆H₄)—O—(CH₂)₁₀—CO-2xgGlu
4-HOOC—(C₆H₄)—O—(CH₂)₁₀—CO-gGlu-3xGly
4-HOOC—(C₆H₄)—O—(CH₂)₁₀—CO-2xgGlu-2xADO
4-HOOC—(C₆H₄)—O—(CH₂)₁₀—CO-gGlu-TtdSuc
4-HOOC—(C₆H₄)—O—(CH₂)₉—CO
4-HOOC—(C₆H₄)—O—(CH₂)₁₀—CO-gGlu-4xADO
4-HOOC—(C₆H₄)—O—(CH₂)₁₀—CO—NH—CH₂—(C₆H₁₀)—CO-gGlu-2xADO
4-HOOC—(C₆H₄)—O—(CH₂)₉—CO-gGlu-2xADO
3-HOOC—(C₆H₄)—O—(CH₂)₉—CO-gGlu-2xADO
3-HO-Isoxazole-(CH₂)₁₂—CO-gGlu-2xADO
HOS(O)₂—(CH2)₁₅—CO-gGlu-2xADO—NH—CH2—(C₆H₄)—CH₂
HOS(O)₂—(CH₂)₁₃—CO-gGlu-2xADO
Tetrazolyl-(CH₂)₁₅—CO—NH—SO₂—(CH₂)₃—CO—ADO—ADO—NH—CH₂—(C₆H₄)—CH₂
Tetrazolyl-(CH₂)₁₂—CO-gGlu-2xADO
Tetrazolyl-(CH₂)₁₅—CO-gGlu-2xADO and
MeS(O)₂NH(CO)NH—(CH₂)₁₂—CO-gGlu-2xADO.

In one embodiment, the substituent is of Formula I wherein Z₁ is Chem. 1: HOOC—(CH₂)ₙ—CO-*, wherein n is 16; Z₂ is a bond; Z₃ is γGlu; two of Z₄, Z₅, Z₆, Z₇, Z₈, Z₉ are Ado and the remaining four are bonds; Z₁₀ is Chem. 14: *—NH—CH₂—(C₆H₄)—CH₂-*.

In one embodiment, the substituent is of Formula I wherein Z₁ is Chem. 1: HOOC—(CH₂)ₙ—CO-*, wherein n is 16; Z₂ is a bond; Z₃ is γGlu; two of Z₄, Z₅, Z₆, Z₇, Z₈, and Z₉ are Ado and the remaining four are bonds; Z₁₀ is a bond.

In one embodiment, the substituent is of Formula I wherein Z₁ is Chem. 1: HOOC—(CH₂)ₙ—CO-*, wherein n is 14 or 16; Z₂ is a bond; Z₃ is γGlu; and all of Z₄, Z₅, Z₆, Z₇, Z₈ and Z₉ are bonds; Z₁₀ is a bond.

In one embodiment, the substituent is of Formula I wherein Z₁ is Chem. 1: HOOC—(CH₂)ₙ—CO-*, wherein n is 16 or 18; Z₂ is Chem 8 (Trx); Z₃ is γGlu; two of Z₄, Z₅, Z₆, Z₇, Z₈ and Z₉ are Ado and the remaining four are bonds; Z₁₀ is a bond.

In one embodiment, the substituent is of Formula I wherein Z₁ is Chem 2: Tetrazolyl-(CH₂)ₙ—CO-*, wherein n is 15; Z₂ is Chem 7 (sulfonimide); Z₃ is a bond; two of Z₄, Z₅, Z₆, Z₇, Z₈ and Z₉ are Ado and the remaining four are bonds; Z₁₀ is Chem. 14: *—NH—CH₂—(C₆H₄)—CH₂-*.

In one embodiment, the substituent is of Formula I wherein Z₁ is Chem 2: Tetrazolyl-(CH₂)ₙ—CO-*, wherein n is 15; Z₂ is a bond; Z₃ is γGlu; two of Z₄, Z₅, Z₆, Z₇, Z₈ and Z₉ are Ado and the remaining four are bonds; Z₁₀ is a bond.

In one embodiment, the substituent is of Formula I wherein Z₁ is Chem 2: Tetrazolyl-(CH₂)ₙ—CO-*, wherein n is 12; Z₂ is a bond; Z₃ is γGlu; two of Z₄, Z₅, Z₆, Z₇, Z₈ and Z₉ are Ado and the remaining four are bonds; Z₁₀ is a bond.

In one embodiment, the substituent is of Formula I wherein Z₁ is Chem. 3: HOOC—(C₆H₄)—O—(CH₂)ₘ—CO-*, wherein m is 10; Z₂ is a bond; Z₃ is a bond; and all off Z₄, Z₅, Z₆, Z₇, Z₈ and Z₉ are bonds; Z₁₀ is a bond.

In one embodiment, the substituent is of Formula I wherein Z₁ is Chem. 3: HOOC—C₆H₄)—O—(CH₂)ₘ—CO-*, wherein m is 10; Z₂ is a bond; Z₃ is a γGlu; and all off Z₄, Z₅, Z₆, Z₇, Z₈ and Z₉ are bonds; Z₁₀ is a bond.

In one embodiment, the substituent is of Formula I wherein Z₁ is Chem. 3: HOOC—(C₆H₄)—O—(CH₂)ₘ—CO-*, wherein m is 10; Z₂ is a bond; Z₃ is a γGlu; and one off Z₄, Z₅, Z₆, Z₇, Z₈ and Z₉ is a γGlu and the remaining five are bonds; Z₁₀ is a bond.

In one embodiment, the substituent is of Formula I wherein Z₁ is Chem. 3: HOOC—(C₆H₄)—O—(CH₂)ₘ—CO-*, wherein m is 10; Z₂ is a bond; Z₃ is a γGlu; and one off Z₄, Z₅, Z₆, Z₇, Z₈ and Z₉ is a γGlu and two are Ado and the remaining three are bonds; Z₁₀ is a bond.

In one embodiment, the substituent is of Formula I wherein Z₁ is Chem. 3: HOOC—(C₆H₄)—O—(CH₂)ₘ—CO-*, wherein m is 10; Z₂ is a bond; Z₃ is a γGlu; and three off Z₄, Z₅ Z₆, Z₇, Z₈ and Z₉ are Gly and the remaining three are bonds; Z₁₀ is a bond.

In one embodiment, the substituent is of Formula I wherein Z₁ is Chem. 3: HOOC—(C₆H₄)—O—(CH₂)ₘ—OC-*, wherein m is 10; Z₂ is a bond; Z₃ is a γGlu; and two off Z₄, Z₅ , Z₆, Z₇, Z₈ and Z₉ are Ado and the remaining four are bonds; Z₁₀ is a bond.

In one embodiment, the substituent is of Formula I wherein Z₁ is Chem. 3: HOOC—(C₆H₄)—O—(CH₂)ₘ—CO-*, wherein m is 10; Z₂ is a bond; Z₃ is a γGlu; and three off Z₄, Z₅ Z₆, Z₇, Z₈ and Z₉ are Ado and the remaining three are bonds; Z₁₀ is a bond.

In one embodiment, the substituent is of Formula I wherein Z₁ is Chem. 3: HOOC—(C₆H₄)—O—(CH₂)ₘ—CO-*, wherein m is 10; Z₂ is a bond; Z₃ is a γGlu; and four off Z_{4,} Z₅, Z₆, Z₇, Z₈ and Z₉ are Ado and the remaining two are bonds; Z₁₀ is a bond.

In one embodiment, the substituent is of Formula I wherein Z₁ is Chem. 3: HOOC—(C₆H₄)—O—(CH₂)ₘ—CO-*, wherein m is 10; Z₂ is a bond; Z₃ is a γGlu; and one off Z₄, Z₅, Z₆, Z₇, Z₈ and Z₉ is a TtdSuc and the remaining five are bonds; Z₁₀ is a bond.

In one embodiment, the substituent is of Formula I wherein Z₁ is Chem. 3: HOOC—(C₆H₄)—O—(CH₂)ₘ—CO-*, wherein m is 10; Z₂ is Chem 8 (Trx); ; Z₃ is a γGlu; and two off Z₄, Z₅ Z₆, Z₇, Z₈ and Z₉ are Ado and the remaining four are bonds; Z₁₀ is a bond.

In one embodiment, the substituent is of Formula I wherein Z₁ is Chem. 3: HOOC—(C₆H₄)—O—(CH₂)ₘ—CO-*, wherein m is 9; Z₂ is a bond; Z₃ is a γGlu; and one off Z₄, Z₅, Z₆, Z₇, Z₈ and Z₉ is a TtdSuc and the remaining five are bonds; Z₁₀ is a bond.

In one embodiment, the substituent is of Formula I wherein Z₁ is Chem. 3: HOOC—(C₆H₄)—O—(CH₂)ₘ—OC-*, wherein m is 10; Z₂ is a bond; Z₃ is γGlu; two of Z₄, Z₅ , Z₆, Z₇, Z₈ and Z₉ are Ado, the remaining four are bonds; Z₁₀ is a bond.

In one embodiment, the substituent is of Formula I wherein Z₁ is Chem. 3: HOOC—(C₆H₄)—O—(CH₂)ₘ—OC-*, wherein m is 10; Z₂ is a bond; Z₃ is γGlu; two of Z₄, Z₅ , Z₆, Z₇, Z₈ and Z₉ are Ado, the remaining four are bonds; Z₁₀ is a bond.

In one embodiment, the substituent is of Formula I wherein Z₁ is Chem. 4: HO—S(O)₂—(CH₂)ₙ—CO-*, wherein n is 15; Z₂ is a bond; Z₃ is γGlu; two of Z₄, Z₅ , Z₆, Z₇, Z₈ and Z₉ are Ado, the remaining four are bonds; Z₁₀ is a bond.

In one embodiment, the substituent is of Formula I wherein Z₁ is Chem. 4: HO—S(O)₂—(CH₂)ₙ—OC-*, wherein n is 15; Z₂ is a bond; Z₃ is γGlu; two of Z₄, Z₅ , Z₆, Z₇, Z₈ and Z₉ are Ado, the remaining four are bonds; Z₁₀ is Chem. 14: *—NH—CH₂—(C₆H₄)—CH₂-*.

In one embodiment, the substituent is of Formula I wherein Z₁ is Chem. 5: MeS(O)₂NH(CO)NH—(CH₂)ₙ—CO-*, wherein n is 12; Z₂ is a bond; Z₃ is γGlu; two of Z₄, Z₅, Z₆, Z₇, Z₈ and Z₉ are Ado, the remaining four are bonds; Z₁₀ is a bond.

In one embodiment, the substituent is of Formula I wherein Z₁ is Chem. 6: 3-OH—Isoxezole-(CH₂)₁₂—CO-*, wherein n is 12; Z₂ is a bond; Z₃ is γGlu; two of Z₄, Z₅, Z₆, Z₇, Z₈ and Z₉ are Ado, the remaining four are bonds; Z₁₀ is a bond.

### Specific Substituent Combinations:

In one embodiment, the compound of the invention comprises or has two substituents of Formula I wherein Z₁ is Chem. 1: HOOC—(CH₂)ₙ—CO-*, wherein n is 16; Z₂ is a bond; Z₃ is γGlu; two of Z₄, Z₅, Z₆, Z₇, Z₈, Z₉ are Ado and the remaining four are bonds; Z₁₀ is a bond.

In one embodiment, the compound of the invention comprises or has two substituents of Formula I wherein Z₁ is Chem. 1: HOOC—(CH₂)ₙ—CO-*, wherein n is 14; Z₂ is a bond; Z₃ is γGlu; two of Z₄, Z₅, Z₆, Z₇, Z₈, Z₉ are Ado and the remaining four are bonds; Z₁₀ is a bond.

In one embodiment, the compound of the invention comprises or has two substituents of Formula I wherein Z₁ is Chem. 1: HOOC—(CH₂)ₙ—CO-*, wherein n is 14; Z₂ is a bond; Z₃ is γGlu; all four of Z₄, Z₅, Z₆, Z₇, Z₈, Z₉ are bonds; Z₁₀ is a bond.

In one embodiment, the compound of the invention comprises or has two substituents of Formula I wherein Z₁ is Chem. 3: HOOC—(C₆H₄)—O—(CH₂)ₘ—CO-*, wherein m is 10; Z₂ is a bond; Z₃ is γGlu; two of Z₄, Z₅ Z₆, Z₇, Z₈ and Z₉ are Ado, the remaining four are bonds; Z₁₀ is a bond.

In one embodiment, the compound of the invention comprises or has two substituents, one being of Formula I wherein Z₁ is Chem. 1: HOOC—(CH₂)ₙ—CO-*, wherein n is 16; Z₂ is a bond; Z₃ is γGlu; two of Z₄, Z₅, Z₆, Z₇, Z₈, Z₉ are Ado and the remaining four are bonds; Z₁₀ is Chem. 14: *—NH—CH₂-(C₆H₄)—CH₂-*; the other substituent being of Formula I wherein Z₁ is Chem. 1: HOOC—(CH₂)ₙ—CO-*, wherein n is 16; Z₂ is a bond; Z₃ is γGlu; two of Z₄, Z₅, Z₆, Z₇, Z₈, Z₉ are Ado and the remaining four are bonds; Z₁₀ is a bond.

In one embodiment, the compound of the invention comprises or has two substituents, one being of Formula I wherein Z₁ is Chem. 1: HOOC—(CH₂)ₙ—CO-*, wherein n is 16; Z₂ is a bond; Z₃ is γGlu; two of Z₄, Z₅, Z₆, Z₇, Z₈, Z₉ are Ado and the remaining four are bonds; Z₁₀ is Chem. 14: *—NH—CH₂—(C₆H₄)—CH₂-*; the other substituent being of Formula I wherein Z₁ is Chem. 3: HOOC—(C₆H₄)—O—-(CH₂)ₘ—CO-*, wherein m is 10; Z₂ is a bond; Z₃ is γGlu; two of Z₄, Z₅, Z₆, Z₇, Z₈ and Z₉ are Ado, the remaining four are bonds; Z₁₀ is a bond.

In one embodiment, the compound of the invention comprises or has two substituents, one being of Formula I wherein Z₁ is Chem. 1: HOOC—(CH₂)ₙ—CO-*, wherein n is 16; Z₂ is a bond; Z₃ is γGlu; two of Z₄, Z₅, Z₆, Z₇, Z₈, Z₉ are Ado and the remaining four are bonds; Z₁₀ is a bond; the other substituent being of Formula I wherein Z₁ is Chem. 3: HOOC—(C₆H₄)—O—(CH₂)ₘ—CO-*, wherein m is 10; Z₂ is a bond; Z₃ is γGlu; two of Z₄, Z₅, Z₆, Z₇, Z₈ and Z₉ are Ado, the remaining four are bonds; Z₁₀ is a bond.

In one embodiment, the compound of the invention comprises or has two substituents, one being of Formula I wherein Z₁ is Chem. 1: HOOC—(CH₂)ₙ—CO-*, wherein n is 16; Z₂ is a bond; Z₃ is γGlu; two of Z₄, Z₅, Z₆, Z₇, Z₈, Z₉ are Ado and the remaining four are bonds; Z₁₀ is a bond; and the other substituent is of formula I wherein Z₁ is Chem. 4: HOS(O)₂—(CH₂)ₙ—CO-*, wherein m is 15; Z₂ is a bond; Z₃ is γGlu; two of Z₄, Z₅, Z₆, Z₇, Z₈ and Z₉ are Ado, the remaining four are bonds; Z₁₀ is Chem. 14: *—NH—CH₂—(C₆H₄)—CH₂-*.

In one embodiment, the compound of the invention comprises or has two substituents, one being of Formula I wherein Z₁ is Chem. 3: HOOC—(C₆H₄)—O—(CH₂)ₘ—CO—*, wherein m is 10; Z₂ is a bond; Z₃ is γGlu; two of Z₄, Z₅, Z₆, Z₇, Z₈ and Z₉ are Ado, the remaining four are bonds; Z₁₀ is a bond; the other substituent being of Formula I wherein Z₁ is Chem. 4: HOS(O)₂—(CH₂)ₙ—CO-*, wherein m is 15; Z₂ is a bond; Z₃ is γGlu; two of Z₄, Z₅, Z₆, Z₇, Z₈ and Z₉ are Ado, the remaining four are bonds; Z₁₀ is Chem. 14: *—NH—CH₂—(C₆H₄)—CH₂-*.

### Peptide and Attachment Site

An EGF(A) derivative or compound according to the invention comprises an EGF(A) peptide analogue of the EGF(A) domain of LDL-R as defined by SEQ ID NO.: 1. Such peptide sequence have been described in details herein above and the peptide of the derivative or compound of the invention may be described and defined by identical terms. The EGF(A) derivative or compound further has at least one substituent as described herein above which is linked to the peptide sequence.

In the compounds of the invention, the substituent is covalently attached to the peptide, meaning to one amino acid residue of the peptide sequence.

In one embodiment the EGF(A) derivative of the invention, comprise a substituent which is not attached to any one of the following positions: 295, 296, 298, 301, 302 and 307.

In a further embodiment the substituent is not attached to any one of the following positions:

295, 296, 298, 301, 302, 307 and 310. In further such embodiments, it is also not attached to any one of the following positions: 299 and 320.

In a particular embodiment a substituent is attached via any position from 292 to 333 except in any or the positions 297, 304, 308, 317, 319 and 331.

In a particular embodiment a substituent attached via any position from 292 to 333 except in any of the positions 297, 298, 301, 302, 304, 307, 308, 317, 319 and 331.

In a particular embodiment a substituent attached via any position from 292 to 333 except in any of the positions 295, 296, 297, 298, 301, 302, 304, 307, 308, 317, 319 and 331. In a particular embodiment a substituent attached via in any position from 292 to 333 except in any of the positions 295, 296, 297, 298, 301, 302, 304, 307, 308, 310, 317, 319, 320 and 331. In a particular embodiment a substituent attached via any position from 292 to 333 except in any of the positions 295, 296, 297, 298, 301, 302, 304, 307, 308, 309, 310, 317, 319, 320 and 331.

In one embodiment, the substituent(s) is/are attached to any one or two of the positions 292, 293, 294, 299, 300, 303, 305, 306, 309, 311, 312, 313, 314, 315, 316, 318, 320, 321, 322, 323, 324, 325, 326, 327, 328, 329, 330, 332 and 333 of the EGF(A) peptide analogue.

In one embodiment, the substitution(s) is/are attached to any one or two of the positions 292, 293, 294, 300, 303, 305, 306, 309, 311, 312, 313, 314, 315, 316, 318, 321, 322, 323, 324, 325, 326, 327, 328, 329, 330, 332 and 333 of the EGF(A) peptide analogue.

In one embodiment, the substitution(s) is/are attached to any one or two of the positions 292, 293, 294, 300, 303, 305, 306, 311, 312, 313, 314, 315, 316, 318, 321, 322, 323, 324, 325, 326, 327, 328, 329, 330, 332 and 333 of the EGF(A) peptide analogue.

In one embodiment, the substituent is attached to the N-terminal amino acid of the peptide sequence. In a particular embodiment, the N-terminal amino acid is Gly. In a particular embodiment, the N-terminal amino acid is 293Gly. In a particular embodiment, the N-terminal amino acid is 293Lys. In a particular embodiment, the N-terminal amino acid is 292Lys. It may also be a Lys or a Gly or another amino acid residue in the N-terminal position which may be 293 or any position further down from the N-terminus, such as 294Thr, 294Gly or 294Lys or 295Asn. In a particular embodiment, the substituent is attached to the alpha-nitrogen of the N-terminal amino acid residue of the peptide analogue. In another embodiment, if the N-terminal amino acid residue is Lys, the substituent may be covalently linked to the alpha-nitrogen or to the epsilon amino group of the lysine residue.

In a particular embodiment, a substituent is attached to the E-amino group of a Lys residue present in the peptide.

In another embodiment, a substituent is attached to a Lys in C-terminal position which may be position 332, 333 or any position further towards the C-terminus.

In embodiments wherein the peptides of the invention comprise an elongation, either in N-terminal or C-terminal, the substituent(s) may be attached to an amino acid residue of said elongation(s). In the presence of a N-terminal elongation, a substituent may be attached to the N-terminal amino acid of said elongation or to a Lys present within the elongation sequence. In the presence of a C-terminal elongation, a substituent may be attached to a Lys residue in C-terminal position or to a Lys present within the elongation sequence.

In yet another embodiment, the substituent is attached to an amino acid present in the peptide sequence. In a particular embodiment, the substituent is linked to a lysine residue present in the peptide. In a particular embodiment, the substituent is linked to the epsilon amino group of a lysine residue present in the peptide. The lysine residue to which the substituent is linked may be located in any position of the LDL-R(293-332) EGF(A) analogue including the N-terminal position or C-terminal position of the peptide, any position within or at the N-terminal end residue of a N-terminal elongation if present, any position within or at the C-terminal end residue of a C-terminal elongation if present.

As described herein above the EGF(A) peptide analogue may have one or more Lys residues; and those residues are useful for attachment of substituents.

In a particular embodiment, the lysine(s) to which the substituent(s) is/are linked is selected from the group of: 292Lys, 293Lys, 294Lys, 299Lys, 300Lys, 303Lys, 305Lys, 306Lys, 309Lys, 311Lys, 312Lys, 313Lys, 314Lys, 315Lys, 316Lys, 318Lys, 320Lys, 321Lys, 322Lys, 323Lys, 324Lys, 325Lys, 326Lys, 327Lys, 328Lys, 329Lys, 330Lys, 332Lys and 333Lys.

In a particular embodiment, the lysine(s) to which the substituent(s) is/are linked is selected from 293Lys, 294Lys, 295Lys, 296Lys, 298Lys, 299Lys, 301Lys, 302Lys, 303Lys, 305Lys, 306Lys, 307Lys, 309Lys, 310Lys, 311Lys, 312Lys, 313Lys, 314Lys, 315Lys, 316Lys, 318Lys, 320Lys, 321Lys, 322Lys, 323Lys, 324Lys, 325Lys, 326Lys, 327Lys, 328Lys, 329Lys, 330Lys, 332Lys and 333Lys.

In a particular embodiment, the lysine(s) to which the substituent(s) is/are linked is/are selected from 293Lys, 294Lys, 300Lys, 303Lys, 306Lys, 309Lys, 311Lys, 312Lys, 313Lys, 314Lys, 315Lys, 316Lys, 318Lys, 321Lys, 322Lys, 323Lys, 324Lys, 325Lys, 326Lys, 328Lys, 329Lys, 330Lys, 332Lys and 333Lys.

In another embodiment, the lysine(s) to which the substituent(s) is/are linked is/are selected from 293Lys, 294Lys, 298Lys, 299Lys, 303Lys, 305Lys, 306Lys, 309Lys, 311Lys, 312Lys, 313Lys, 314Lys, 315Lys, 316Lys, 318Lys, 320Lys, 321Lys, 322Lys, 323Lys, 324Lys, 325Lys, 326Lys, 327Lys, 328Lys, 329Lys, 330Lys, 332Lys and 333Lys.

In another embodiment, the lysine(s) to which the substituent(s) is/are linked is/are selected from: 292Lys, 293Lys, 294Lys, 299Lys, 300Lys, 303Lys, 305Lys, 306Lys, 309Lys, 311Lys, 313Lys, 314Lys, 315Lys, 316Lys, 318Lys, 320Lys, 321Lys, 322Lys, 323Lys, 324Lys, 325Lys, 326Lys, 327Lys, 328Lys, 329Lys, 330Lys, 332Lys and 333Lys.

In another embodiment, the lysine(s) to which the substituent(s) is/are linked is/are selected from: 292Lys, 293Lys, 294Lys, 300Lys, 303Lys, 305Lys, 306Lys, 309Lys, 311Lys, 313Lys, 314Lys, 316Lys, 318Lys, 321Lys, 322Lys, 323Lys, 324Lys, 325Lys, 326Lys, 327Lys, 328Lys, 329Lys, 330Lys, 332Lys and 333Lys.

In another embodiment, the lysine(s) to which the substituent(s) is/are linked is/are selected from: 293Lys, 294Lys, 300Lys, 303Lys, 305Lys, 306Lys, 309Lys, 311Lys, 313Lys, 314Lys, 316Lys, 318Lys, 321Lys, 322Lys, 323Lys, 324Lys, 325Lys, 326Lys, 327Lys, 328Lys, 329Lys, 330Lys, 332Lys and 333Lys.

In another embodiment, the lysine(s) to which the substituent(s) is/are linked is/are selected from: 293Lys, 294Lys, 300Lys, 303Lys, 305Lys, 306Lys, 311Lys, 313Lys, 314Lys, 316Lys, 318Lys, 321Lys, 322Lys, 323Lys, 324Lys, 325Lys, 326Lys, 327Lys, 328Lys, 329Lys, 330Lys, 332Lys and 333Lys.

In embodiments where the substituent is attached to a C-terminal elongation, the lysine to which the substituent is linked may be selected from anyone of 333Lys to 242Lys position and/or to anyone of 333Lys to 383Lys position.

In embodiments where compounds of the invention have two substituents, the substituents may be linked independently of each other as defined above, meaning that either one may be attached to the N-terminal amino acid of the peptide, to the C-terminal amino acid of the peptide, or to an amino acid within the amino acid sequence of the peptide.

In embodiments where a Lys is present in N-terminal position, two substituents may be both linked to the N-terminal Lys of the peptide. One may be linked to the N-terminal alpha-amine of said Lys while the other may be linked to the epsilon nitrogen of said Lys. When two substituents are present, one may be linked to the N-terminal amino acid of the peptide while the other substituent is linked to an amino acid, such as a Lys, within the peptide. Alternatively, one substituent may be linked to a Lys in position C-terminal of the peptide while the other substituent is linked to an amino acid, such as a Lys, in the peptide. Alternatively, one substituent may be linked to an amino acid residue, such as a Lys, within the peptide, including elongations, the other substituent being linked to another amino acid residue, such as a Lys, within the peptide, including elongations.

In an embodiment, the compounds of the invention have one substituent, said substituent is linked to the peptide at the N-terminal; or said substituent is linked to the peptide in position 292Lys; or said substituent is linked to the peptide in position 293Lys, or said substituent is linked to the peptide in position 299Lys; or said substituent is linked to the peptide in position 300Lys; or said substituent is linked to the peptide in position 309Lys; or said substituent is linked to the peptide in position 311Lys; or said substituent is linked to the peptide in position 312Lys; or said substituent is linked to the peptide in position 313Lys; or said substituent is linked to the peptide in position 314Lys; or said substituent is linked to the peptide in position 315Lys; or said substituent is linked to the peptide in position 316Lys; or said substituent is linked to the peptide in position 318Lys; or said substituent is linked to the peptide in position 320Lys; or said substituent is linked to the peptide in position 321Lys; or said substituent is linked to the peptide in position 322Lys; or said substituent is linked to the peptide in position 323Lys; or said substituent is linked to the peptide in position 324Lys; or said substituent is linked to the peptide in position 325Lys; or said substituent is linked to the peptide in position 326Lys; or said substituent is linked to the peptide in position 328Lys; or said substituent is linked to the peptide in position 329Lys; or said substituent is linked to the peptide in position 330Lys; or said substituent is linked to the peptide in position 332Lys; or said substituent is linked to the peptide in position 333Lys.

In an embodiment where the derivative of the invention has two substituents, said substituents may be linked to the peptide via the N-terminal and any of the above mention Lys positions, such as 293Lys, 309Lys, 313Lys, 324Lys, 328Lys, 330Lys, 332Lys and 333Lys.

In further embodiments where the derivative comprises two substituents, they may be linked to two different Lys residues, such as any of the following pairs of Lys residues

| | | | |
|---|---|---|---|
| i. | 293K and 294K | xiv. | 313K and 321K |
| ii. | 293K and 312K | xv. | 313K and 324K |
| iii. | 293K and 333K | xvi. | 313K and 328K |
| iv. | 309K and 313K | xvii. | 313K and 332K |
| v. | 309K and 324K | xviii. | 313K and 333K |
| vi. | 309K and 328K | xix. | 314K and 333K |
| vii. | 309K and 332K | xx. | 321K and 332K |
| viii. | 309K and 333K | xxi. | 321Kand 333K |
| ix. | 311Kand 313K | xxii. | 324K and 333K |
| x. | 312K and 333K | xxiii. | 324K and 328K |
| xi. | 312K and 313K | xxiv. | 328K and 333K |
| xii. | 312K and 314K | xxv. | 330K and 333K and |
| xiii. | 313K and 314K | xxvi. | 332K and 333K. |

In one embodiment the two substituents are attached via 333Lys and a Lys selected from 293Lys, 309Lys, 312Lys, 313Lys, 314Lys, 321Lys, 324Lys, 328Lys, 330Lys and 332Lys.

In one embodiment the two substituents are attached via 333Lys and a Lys selected from 312Lys, 313Lys, 314Lys, 321Lys, 324Lys, 328Lys and 330Lys.

In one embodiment the two substituents are attached via 333Lys and a Lys selected from 313Lys, 324Lys and 328Lys.

As described above the peptide may have one or more amino acid substitutions which may be combined with specific amino acid residues in specific positions as described herein. Such specific amino acid residues may be wild type amino acid residues that should be maintained, such as the cysteines which may in a series of preferred embodiments e.g. in combination with other features described herein, be present in the peptide analogue. In such embodiments the peptide analogue comprises three disulphide bridges in positions 297Cys-308Cys, 304Cys-317Cys and 319Cys-331Cys. In a further example of such embodiments the peptide analogue of a peptide derivative comprises three disulphide bridges in positions 297Cys-308Cys, 304Cys-317Cys and 319Cys-331Cys and at least one substituent, wherein the substituent(s) is not attached to a positions selected from 295, 296, 298, 301, 302 and 307 of said peptide analogue, The skilled person will understand that combinations of peptide sequence information may be combined with information on position and identity of the substituent to define various specific embodiments of the present invention.

In an embodiment, the peptide analogue comprises no Lys in other positions than the positions to which a substituent is linked.

In an embodiment, the compounds of the invention have one substituent, said substituent is linked either in position N-terminal or to a Lys in any position, and the peptide analogue comprises no Lys in all other positions. In an embodiment, the compounds of the invention have one substituent, said substituent is linked to a Lys in any position other than position 312, and the peptide analogue comprises an Arg in position 312Arg.

In an embodiment, the compounds of the invention have two substituents, and the peptide analogue comprises no Lys in positions other than positions to which the substituents are linked.

In one embodiment the EGF(A) derivative according to the invention is selected from the group of EGF(A) derivative consisting of: Examples 1-47, 51-102 and 106-159 disclosed in WO2017/121850.

In further embodiments the EGF(A) derivative according to the invention is individually selected from the group of EGF(A) derivative consisting of: Examples 1-47, 51-102 and 106-159 disclosed in WO02017/121850.

In one embodiment the EGF(A) derivative according to the invention is selected from the group of EGF(A) derivative consisting of: Examples 1-44, 46-47, 51-55, 57, 60-64, 66-69, 71-102 and 106-159 disclosed in WO2017/121850.

In one embodiment the EGF(A) derivative according to the invention is selected from the group of EGF(A) derivative consisting of: Examples 31, 95, 128, 133, 143, 144, 150, 151, 152 and 153 disclosed in WO2017/121850 with the structure shown below.

| # | Structure |
|---|---|
| 31 | 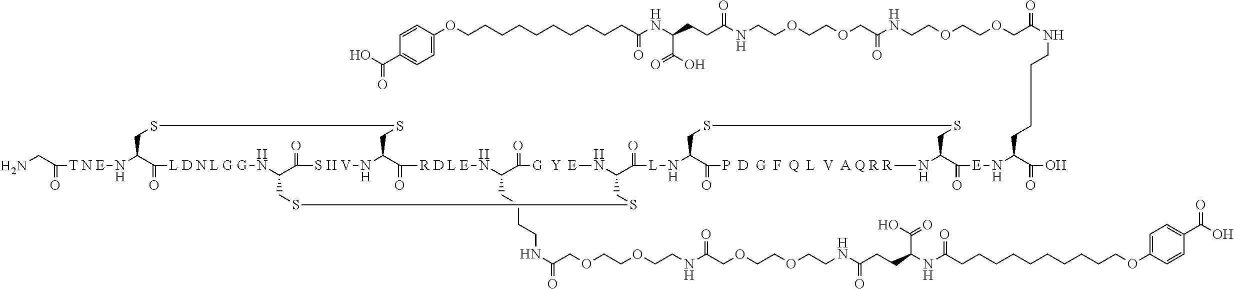 |
| 95 | 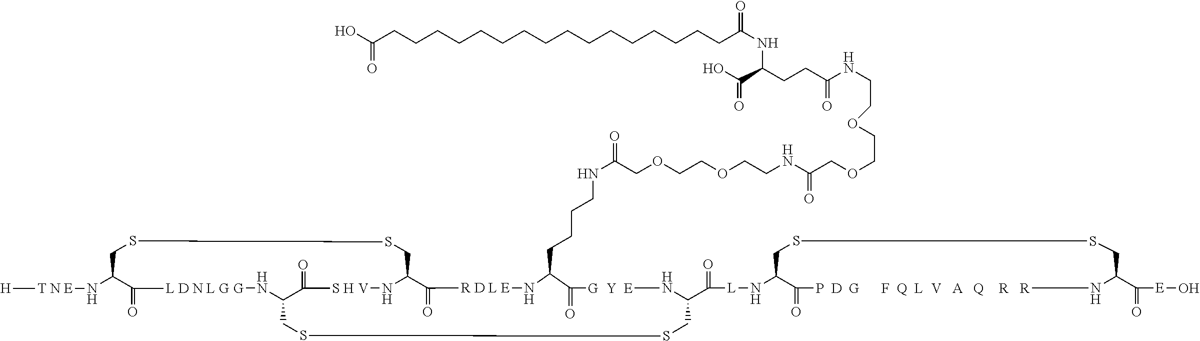 |
| 128 | 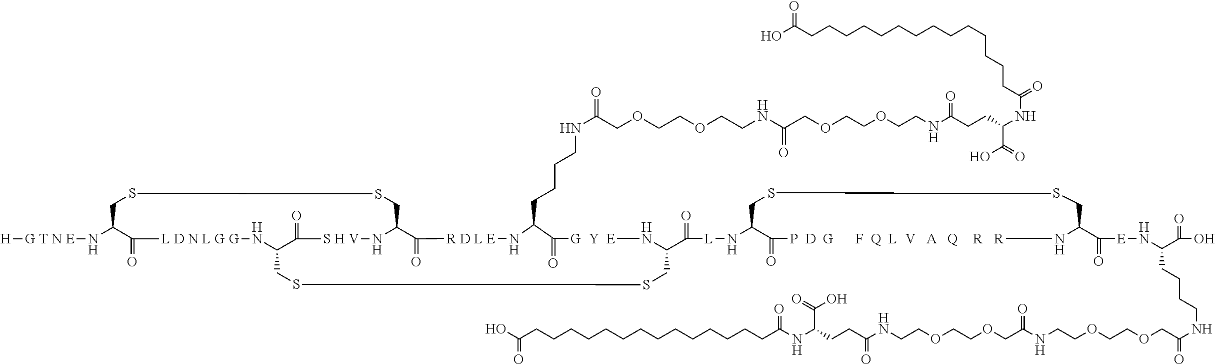 |
| 133 | 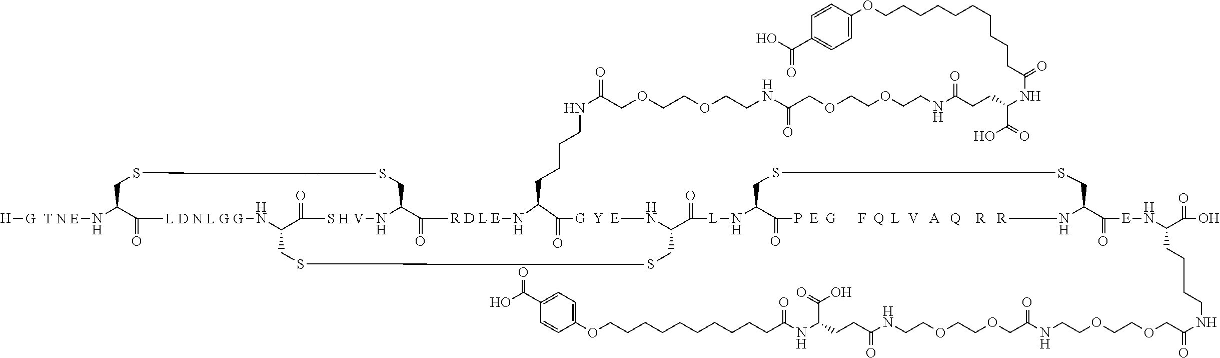 |
| 143 | 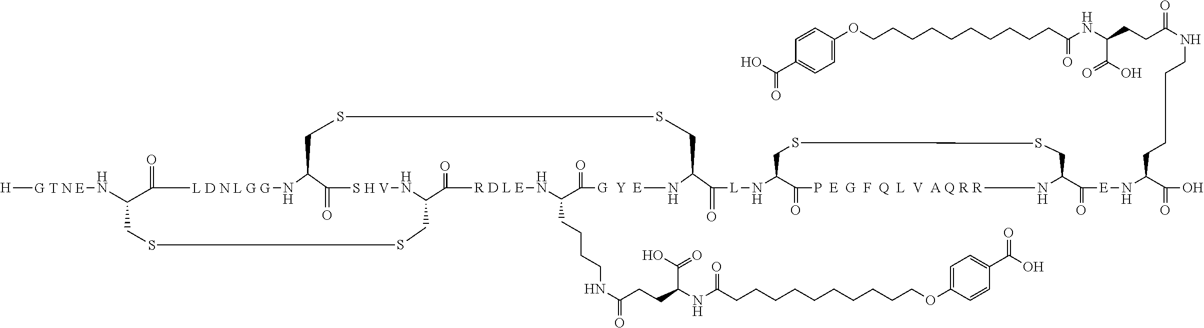 |
| 144 | 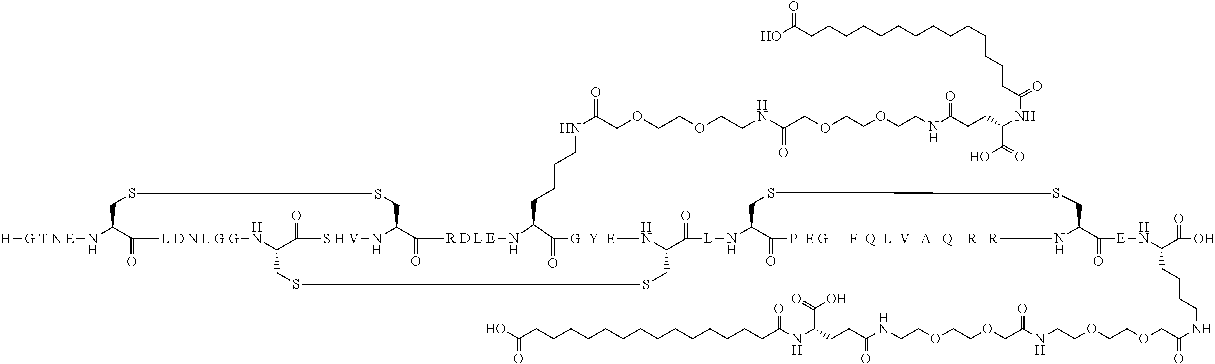 |
| 150 | 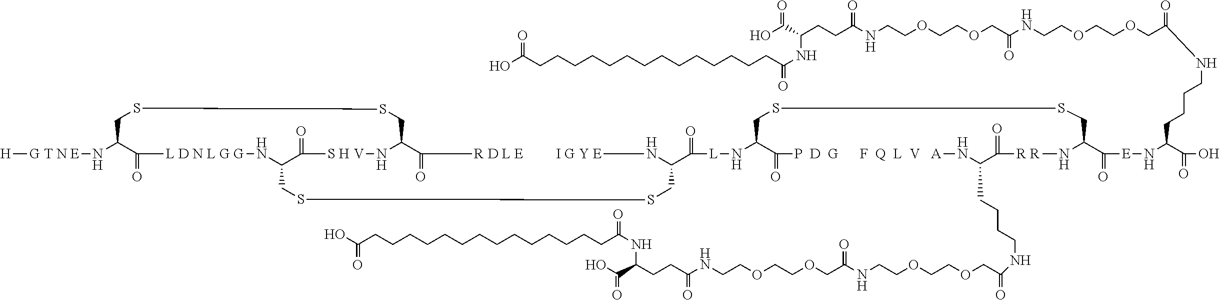 |
| 151 | 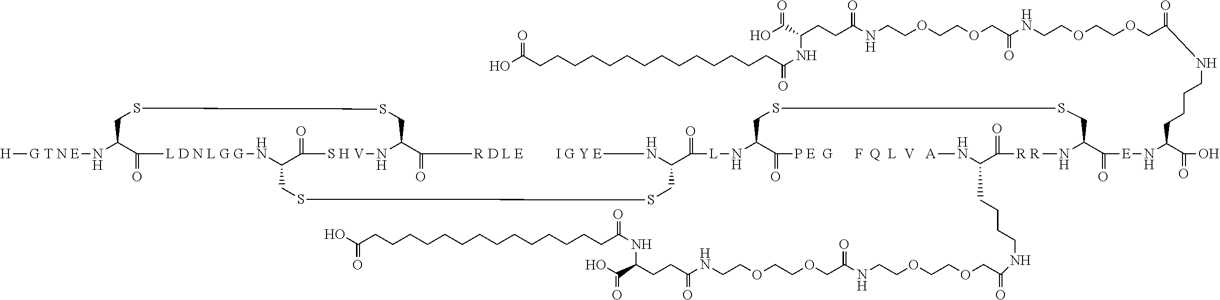 |
| 152 | 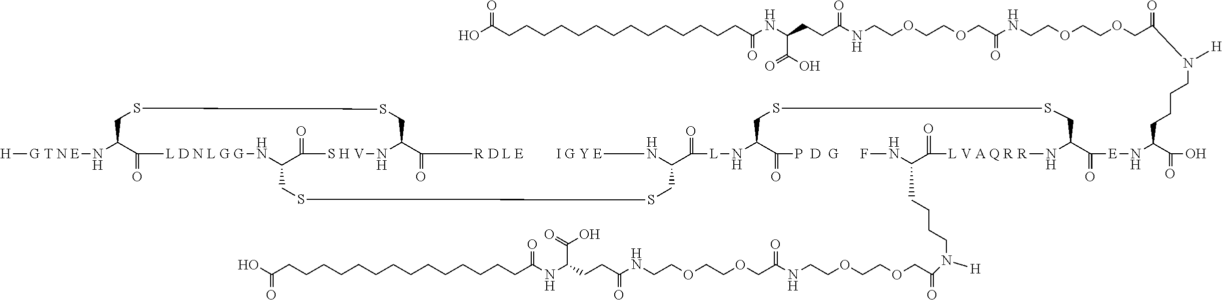 |
| 153 | 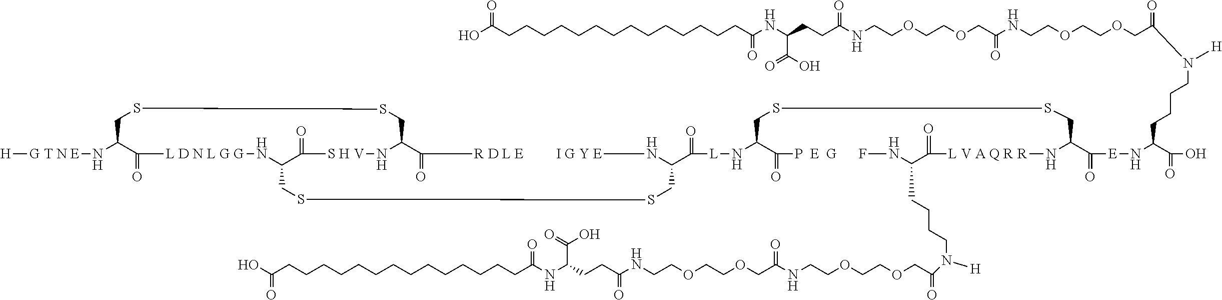 |

### Delivery Agent

### Salt of N-(8-(2-Hydroxybenzoyl)Amino)Caprylic Acid

The delivery agent used in the present invention is a salt of N-(8-(2-hydroxybenzoyl)amino)caprylic acid (NAC). The structural formula of N-(8-(2-hydroxybenzoyl)amino)caprylate is shown in formula (I).

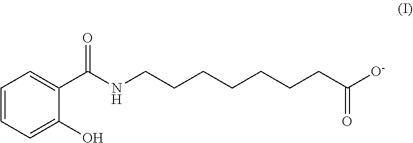 [SMILES: O=C([O-])CCCCCCCNC(=O)c1ccccc1O]

In some embodiments the salt of N-(8-(2-hydroxybenzoyl)amino)caprylic acid comprises one monovalent cation, two monovalent cations or one divalent cation. In some embodiments the salt of N-(8-(2-hydroxybenzoyl)amino)caprylic acid is selected from the group consisting of the sodium salt and potassium salt of N-(8-(2-hydroxybenzoyl)amino)caprylic acid. In one embodiment the salt of N-(8-(2-hydroxybenzoyl)amino)caprylic acid is selected from the group consisting of the sodium salt, potassium salt and/or the ammonium salt. In one embodiment the salt of N-(8-(2-hydroxybenzoyl)amino)caprylic acid is the sodium salt or the potassium salt. In one embodiment the salt of N-(8-(2-hydroxybenzoyl)amino)caprylic acid is selected from the group consisting of the sodium salt and the ammonium salt. Salts of N-(8-(2-hydroxybenzoyl)amino)caprylate may be prepared using the method described in e.g. WO96/030036, WO00/046182, WO01/092206 or WO2008/028859.

The salt of N-(8-(2-hydroxybenzoyl)amino)caprylic acid may be crystalline and/or amorphous. In some embodiments the delivery agent comprises anhydrate, monohydrate, dihydrate, trihydrate, a solvate or one third of a hydrate of the salt of N-(8-(2-hydroxybenzoyl)amino)caprylic acid as well as combinations thereof. In some embodiments the delivery agent is a salt of N-(8-(2-hydroxybenzoyl)amino)caprylic acid as described in WO2007/121318.

In some embodiments the delivery agent is sodium N-(8-(2-hydroxybenzoyl)amino)caprylate (referred to as “SNAC” herein), also known as sodium 8-(salicyloylamino)octanoate.

### Composition

The composition or pharmaceutical composition of the present invention is a solid or dry composition suited for administration by the oral route as described further herein below.

In some embodiments the composition comprises at least one pharmaceutically acceptable excipient. The term “excipient” as used herein broadly refers to any component other than the active therapeutic ingredient(s) or active pharmaceutical ingredient(s) (API(s)). An excipient may be a pharmaceutically inert substance, an inactive substance, and/or a therapeutically or medicinally none active substance.

The excipients may serve various purposes, e.g. as a carrier, vehicle, filler, binder, lubricant, glidant, disintegrant, flow control agent, crystallization inhibitors, solubilizer, stabilizer, colouring agent, flavouring agent, surfactant, emulsifier or combinations of thereof and/or to improve administration, and/or absorption of the therapeutically active substance(s) or active pharmaceutical ingredient(s). As described herein the salt of N-(8-(2-hydroxybenzoyl)amino)caprylic acid is an excipient acting as a delivery agent. The amount of each excipient used may vary within ranges conventional in the art. Techniques and excipients which may be used to formulate oral dosage forms are described in Handbook of Pharmaceutical Excipients, 8th edition, Sheskey et al., Eds., American Pharmaceuticals Association and the Pharmaceutical Press, publications department of the Royal Pharmaceutical Society of Great Britain (2017); and Remington: the Science and Practice of Pharmacy, 22nd edition, Remington and Allen, Eds., Pharmaceutical Press (2013).

In some embodiments the excipients may be selected from binders, such as polyvinyl pyrrolidone (povidone), etc.; fillers such as cellulose powder, microcrystalline cellulose, cellulose derivatives like hydroxymethylcellulose, hydroxyethylcellulose, hydroxypropylcellulose and hydroxy-propylmethylcellulose, dibasic calcium phosphate, corn starch, pregelatinized starch, etc.; lubricants and/or glidants such as stearic acid, magnesium stearate, sodium stearylfumarate, glycerol tribehenate, etc.; flow control agents such as colloidal silica, talc, etc.; crystallization inhibitors such as povidone, etc.; solubilizers such as pluronic, povidone, etc.; colouring agents, including dyes and pigments such as iron oxide red or yellow, titanium dioxide, talc, etc.; pH control agents such as citric acid, tartaric acid, fumaric acid, sodium citrate, dibasic calcium phosphate, dibasic sodium phosphate, etc.; surfactants and emulsifiers such as pluronic, polyethylene glycols, sodium carboxymethyl cellulose, polyethoxylated and hydrogenated castor oil, etc.; and mixtures of two or more of these excipients and/or adjuvants.

The composition may comprise a binder, such as povidone; starches; celluloses and derivatives thereof, such as microcrystalline cellulose, e.g., Avicel PH from FMC (Philadelphia, Pa.), hydroxypropyl cellulose hydroxylethyl cellulose and hydroxylpropylmethyl cellulose METHOCEL from Dow Chemical Corp. (Midland, Mich.); sucrose; dextrose; corn syrup; polysaccharides; and gelatine. The binder may be selected from the group consisting of dry binders and/or wet granulation binders. Suitable dry binders are, e.g., cellulose powder and microcrystalline cellulose, such as Avicel PH 102 and Avicel PH 200. In some embodiments the composition comprises Avicel, such as Avicel PH 102. Suitable binders for wet granulation or dry granulation are corn starch, polyvinyl pyrrolidone (povidone), vinylpyrrolidone-vinylacetate copolymer (copovidone) and cellulose derivatives like hydroxymethylcellulose, hydroxyethylcellulose, hydroxypropylcellulose and hydroxyl-propylmethylcellulose. In some embodiments the composition comprises povidone.

In some embodiments the composition comprises a filler, which may be selected from lactose, mannitol, erythritol, sucrose, sorbitol, calcium phosphate, such as calciumhydrogen phosphate, microcrystalline cellulose, powdered cellulose, confectioners sugar, compressible sugar, dextrates, dextrin and dextrose. In some embodiments the composition comprises microcrystalline cellulose, such as Avicel PH 102 or Avicel PH 200.

In some embodiments the composition comprises a lubricant and/or a glidant. In some embodiments the composition comprises a lubricant and/or a glidant, such as talc, magnesium stearate, calcium stearate, zinc stearate, glyceryl behenate, glyceryl dibehenate, behenoyl polyoxyl-8 glycerides, polyethylene oxide polymers, sodium lauryl sulfate, magnesium lauryl sulfate, sodium oleate, sodium stearyl fumarate, stearic acid, hydrogenated vegetable oils, silicon dioxide and/or polyethylene glycol etc. In some embodiments the composition comprises magnesium stearate or glyceryl dibehenate (such as the product Compritol® 888 ATO which consists of mono-, di- and triesters of behenic acid (C22) with the diester fraction being predominant).

In some embodiments the composition comprises a disintegrant, such as sodium starch glycolate, polacrilin potassium, sodium starch glycolate, crospovidon, croscarmellose, sodium carboxymethylcellulose or dried corn starch.

The composition may comprise one or more surfactants, for example a surfactant, at least one surfactant, or two different surfactants. The term “surfactant” refers to any molecules or ions that are comprised of a water-soluble (hydrophilic) part, and a fat-soluble (lipophilic) part. The surfactant may e.g. be selected from the group consisting of anionic surfactants, cationic surfactants, nonionic surfactants, and/or zwitterionic surfactants.

The compositions of the invention have a very high content of the delivery agent. This very high content can be defined relative to the full content of the tablets including also the active pharmaceutical ingredient (i.e. the PCSK9 inhibitor) or alternatively relative to the total content of excipients excluding the active pharmaceutical ingredient. The description here below also refers to compositions consisting of specific ingredients, the PCSK9 inhibitor and excipients, the term consisting is to be understood to never the less encompass trace amounts of any substance with no effect on the function of the composition, which may also be referred to as consisting essential of. Such substances can be impurities remaining in preparation of the PCSK9 inhibitor or from the production of the salt of NAC or minimal amounts of any pharmaceutical acceptable excipient that do not affect the quality or absorption of the formulation.

An aspect of the invention relates to a pharmaceutical composition comprising
a) a PCSK9 inhibitor and
b) a salt of N-(8-(2-hydroxybenzoyl)amino)caprylic acid (NAC) wherein said salt of N-(8-(2-hydroxybenzoyl)amino)caprylic acid (NAC) constitutes at least or above 60 w/w % of the composition.

In further such embodiments the salt of NAC constitutes above 70 w/w %, such as above w/w 75%, such as above 80 w/w %, such as above 85 w/w %, such as above 90 w/w of said composition.

In further such embodiments the salt of NAC constitutes at least 70 w/w %, such as at least 75 w/w %, such as at least 80 w/w %, such as at least 85 w/w %, such as at least 90 w/w % of said composition.

In one embodiment the pharmaceutical composition comprises
a) a PCSK9 inhibitor and
b) a salt of N-(8-(2-hydroxybenzoyl)amino)caprylic acid (NAC), wherein said salt of N-(8-(2-hydroxybenzoyl)amino)caprylic acid (NAC) constitutes at least 90 w/w % of the excipients of the composition.

In one embodiment the pharmaceutical composition consists of
a) a PCSK9 inhibitor and
b) excipients, wherein the excipients are
   i. a salt of N-(8-(2-hydroxybenzoyl)amino)caprylic acid (NAC) and
   ii. one or more further excipients
      wherein said salt of N-(8-(2-hydroxybenzoyl)amino)caprylic acid (NAC) constitutes at least 90 w/w % of the excipients of the composition.

In further such embodiments the salt of NAC constitutes at least at least 91 w/w %, such as at least 92 w/w %, such as at least 93 w/w %, such as at least 94 w/w %, such as at least 95 w/w % of the excipients of the composition.

In further such embodiments the salt of NAC constitutes above 95 w/w %, such as above 96 w/w %, such as above 97 w/w % or such as above 98 w/w % of the excipients of the composition.

An aspect of the invention relates to a pharmaceutical composition comprising
a) a PCSK9 inhibitor and
b) a salt of N-(8-(2-hydroxybenzoyl)amino)caprylic acid (NAC), wherein said salt of N-(8-(2-hydroxybenzoyl)amino)caprylic acid (NAC) constitutes at least 90 w/w %, such as at least 95 w/w % of the excipients of the composition.

In one embodiment the pharmaceutical composition consists of
a) a PCSK9 inhibitor and
b) excipients, wherein the excipients are
   i. a salt of N-(8-(2-hydroxybenzoyl)amino)caprylic acid (NAC) and
   ii. one or more further excipients
      wherein said salt of N-(8-(2-hydroxybenzoyl)amino)caprylic acid (NAC) constitutes at least 90 w/w %, such as at least 95 w/w % of the excipients of the composition.

In further such embodiments the salt of NAC constitutes at least at least 91 w/w %, such as at least 92 w/w %, such as at least 93 w/w %, such as at least 94 w/w %, such as at least 95 w/w % of the excipients of the composition.

In further such embodiments the salt of NAC constitutes above 95 w/w %, such as above 96 w/w %, such as above 97 w/w % or such as above 98 w/w % of the excipients of the composition.

In further such embodiments the salt of NAC constitutes above 60 w/w %, such as above 70 w/w %, such as above 75 w/w % or such as above 80 w/w % of the composition.

In further such embodiments the salt of NAC constitutes at least 60 w/w %, such as at least 70 w/w %, such as at least 75 w/w % or such as at least 80 w/w % of the composition.

In further such embodiments the salt of NAC constitutes at least 95 w/w %, such as at least 96 w/w %, such as at least 97 w/w % or such as at least 98 w/w % of the excipients of the composition.

As mentioned above, the content of excipients, besides the delivery agent is according to the invention preferably minimal. In one embodiment, the pharmaceutical composition comprises at least one lubricant.

In one embodiment the pharmaceutical composition comprises or consists of:
a) a PCSK9 inhibitor,
b) a salt of N-(8-(2-hydroxybenzoyl)amino)caprylic acid (NAC) and
c) at least one lubricant.

In such embodiments the lubricant may be magnesium stearate or glyceryl dibehenate. In one embodiment the lubricant is magnesium stearate. In one embodiment the lubricant is glyceryl dibehenate.

A composition as described above wherein said salt of N-(8-(2-hydroxybenzoyl)amino)caprylic acid (NAC) constitutes at least 95 w/w % of the excipients of the composition may further be a composition wherein said salt constitutes at least or above 60 w/w % of the composition.

Likewise, the compositions described above wherein said salt constitutes at least or above 60 w/w % of the composition may further be a composition wherein said salt of N-(8-(2-hydroxybenzoyl)amino)caprylic acid (NAC) constitutes at least 90, such as at least 95 w/w of the excipients of the composition.

The pharmaceutical composition may further be a composition wherein the salt of N-(8-(2-hydroxybenzoyl)amino)caprylic acid (NAC) is selected from the group consisting of the sodium salt, potassium salt of N-(8-(2-hydroxybenzoyl)amino)caprylic acid (NAC) or alternatively from the group consisting of just the sodium salt and the potassium salt. In one embodiment the salt of N-(8-(2-hydroxybenzoyl)amino)caprylic acid (NAC) is sodium N-(8-(2-hydroxybenzoyl)amino)caprylate.

In embodiments wherein said salt of N-(8-(2-hydroxybenzoyl)amino)caprylic acid (NAC) constitutes at least 90 w/w % of the excipients of the composition, any further excipients constitute at most 10 w/w % of the excipients, i.e. any such binder, filler, and/or lubricant/glidant constitutes at most 10 w/w % of the weight of excipients of the composition. In some embodiments the excipients of the composition comprise at least or above 90 w/w delivery agent, and less than 5 w/w % of any further excipients, such as binder, filler, and/or lubricant/glidant. In one embodiment the excipients of the composition comprise at least 90 w/w % delivery agent and up to or less than 5 w/w % lubricant. In one embodiment the excipients of the composition comprise at least 90 w/w % delivery agent and less than 3 w/w lubricant.

In some embodiments the excipients of the composition comprise at least or above 90 w/w % delivery agent and 0.1-10 w/w %, such as 0.5-8 w/w %, such as 1-5 w/w %, of lubricant. In further such embodiments the excipients of the composition comprise 1-3 w/w or such as 2-2.5 w/w % of lubricant.

In embodiments wherein said salt of N-(8-(2-hydroxybenzoyl)amino)caprylic acid (NAC) constitutes at least 95 w/w % of the excipients of the composition, any further excipients of the composition constitute at most 5 w/w % of the excipients, i.e. any such as binder, filler, and/or lubricant/glidant constitutes at most 5 w/w % of the weight of the excipients of the composition. In some embodiments the excipients of the composition comprise at least 95 w/w % delivery agent and less than 5 w/w % lubricant. In one embodiment the excipients of the composition comprise at least 95 w/w % delivery agent and less than 3 w/w % lubricant.

In some embodiments the excipients of the composition comprise at least 95 w/w delivery agent and 0.1-5 w/w %, such as 0.5-4 w/w % or 1-3 w/w %, of lubricant. In further such embodiments the excipients of the composition comprise 2-2.5 w/w % of lubricant.

The pharmaceutical composition according to the invention is preferably produced in a dosage form suitable for oral administration as described herein below. In the following the absolute amounts of the ingredients of the composition of the invention are provided with reference to the content in a dosage unit i.e. per tablet, capsule or sachet.

The pharmaceutical compositions of the invention may in a further embodiment comprise at most 1000 mg of said salt of N-(8-(2-hydroxybenzoyl)amino)caprylic acid per dose unit. In one embodiment the invention relates to a composition wherein a dose unit comprises at most 600 mg of said salt.

In some embodiments the amount of the salt of N-(8-(2-hydroxybenzoyl) amino)caprylic acid per dose unit is at least 0.05 mmol, such as at least 0.075 mmol, such as at least 0.1 mmol, such as at least 0.125 mmol, such as at least 0.15 mmol, such as at least 0.20 mmol, at least 0.25 mmol, at least 0.30 mmol, at least 0.35 mmol, at least 0.40 mmol, at least 0.45 mmol, at least 0.50 mmol, at least 0.55 mmol or at least 0.60 mmol.

In some embodiments the amount of the salt of N-(8-(2-hydroxybenzoyl)amino)caprylic acid per dosage unit of the composition is up to 3 mmol, such as up to 2.75 mmol, such as up to 2.5 mmol, such as up to 2.25 mmol, such as 2 mmol, such as up to 1.5 mmol, up to 1 mmol, up to 0.75 mmol, up to 0.6 mmol, up to 0.5 mmol, up to 0.4 mmol, up to 0.3 mmol and up to 0.2 mmol.

In some embodiments the amount of the salt of N-(8-(2-hydroxybenzoyl) amino)caprylic acid per dose unit of the composition is in the range of 0.05-3 mmol, 0.10-2.5 mmol, 0.15-2.0 mmol, 0.20-1.5 mmol, 0.25-1.0 mmol, 0.30-0.75 mmol or such as 0.45-0.65 mmol.

In some embodiments, wherein the salt of NAC is SNAC, the amount of SNAC in the composition is at least 20 mg, such as at least 25 mg, such as at least 50 mg, such as at least 75 mg, at least 100 mg, at least 125 mg, at least 150 mg, at least 175 mg, at least 200 mg, at least 225 mg, at least 250 mg, at least 275 mg and at least 300 mg per dose unit.

In some embodiments, wherein the salt of NAC is SNAC, the amount of SNAC in the composition is up to 1000 mg, such as up to 800 mg, such as up to 600 mg, such as up to 575 mg, such as up to 550 mg, up to 525 mg, up to 500 mg, up to 475 mg, up to 450 mg, up to 425 mg, up to 400 mg, up to 375 mg, up to 350 mg, up to 325 mg per dose unit, or up to 300 mg per dose unit.

In some embodiments, wherein the salt of NAC is SNAC, the amount of SNAC in the composition is in the range of 100-1000 mg, such as 150-800 mg, such as 200-750 mg, such as 250-700 mg, such as 300-700 mg, such as 350-650 mg or such as from around 400 to around 600 mg per dose unit, such as around 500 mg per dose unit.

In some embodiments, wherein the salt of NAC is SNAC, the amount of SNAC in the composition is in the range of 20-800 mg, such as 25-600 mg, such as 50-500 mg, such as 50-400 mg, such as 75-400 mg, such as 80-350 mg or such as from around 100 to around 300 mg per dose unit.

In one embodiment, wherein the salt of NAC is SNAC, the amount of SNAC is in the range of 200-800 mg, such as 250-400 mg, such as 250-350 mg, such as 275-325 mg, such as around 300 mg per dose unit.

In one embodiment, wherein the salt of NAC is SNAC, the amount of SNAC is in the range of 20-200 mg, such as 25-175 mg, such as 75-150 mg, such as 80-120 mg such as around 100 mg per dose unit.

In an embodiment, a dose unit of the pharmaceutical compositions of the invention comprises 0.5-150 mg, 0.1-100 mg or 0.2 to 100 mg of the PCSK9 inhibitor.

In some embodiments, wherein the PCSK9 inhibitor is an EGF(A) derivative, a dose unit of the composition comprises an amount of PCSK9 inhibitor is in the range of 0.5-150, 0.5-120, 0.5-100 mg,1-80 mg, 1-70 mg, 1-60, 1-50 mg or 1- 40 mg.

In further such embodiments a dose unit comprises 1-50 mg of the PCSK9 inhibitor, such as 0.75- 40 mg, such as 10, 15, 20, 25 or 30 mg or 35, 40, 45 mg, such as 10-30 or 30-50 mg of the PCSK9 inhibitor per dose unit.

In further such embodiments a dose unit comprises 20 to 150 mg of the PCSK9 inhibitor, such as 20-120 mg, such as 20-100 mg, such as 20-80 mg, such as 20, 30, 40, 50, 60, 70 or 80 mg, such as 20, 30, 40 or 50 mg, or such as 80, 85, 90, 95 or 100 mg, or such as 100, 110, 120 or 130 mg, or such as 50 mg or such as 75 mg of the PCSK9 inhibitor per dose unit.

In further such embodiments a dose unit comprises 5 to 50 mg of the PCSK9 inhibitor, such as 10-45 mg, such as 20, 30 or 40 mg, or such as 25, 35, or 45 mg, or such as 30-50 mg or such as 20-40 mg of the PCSK9 inhibitor per dose unit.

The amount of PCSK9 inhibitor may be varied depending on identity of the PCSK9 inhibitor.

In a preferred embodiment a unit dose of the composition comprises 0.5-50 mg magnesium stearate, such as 1-25 mg, such as 1-10 mg, such as 2-8 mg or such as 2-5 mg magnesium stearate.

In a preferred embodiment the amount of magnesium stearate is determined relative to the amount of the salt of NAC, such as SNAC, such that a unit dose of the composition comprises 1-10 mg, such as 1-8 mg, such as 2-5 mg or such as 2-3 mg magnesium stearate per 100 mg salt of N-(8-(2-hydroxybenzoyl)amino)caprylic acid, such as SNAC.

In a preferred embodiment a unit dose of the composition comprises 80-1000 mg SNAC, 0.5-100 mg PCSK9 inhibitor and 1-50 mg lubricant.

In a preferred embodiment a unit dose of the composition comprises 80-800 mg SNAC, 1.0-80 mg PCSK9 inhibitor and 1-40 mg lubricant.

In a preferred embodiment a unit dose of the composition comprises 100-800 mg SNAC, 2-50 mg PCSK9 inhibitor and 1-40 mg lubricant.

In a preferred embodiment a unit dose of the composition comprises 100-600 mg SNAC, 5-50 mg PCSK9 inhibitor and 1-30 mg lubricant.

In a preferred embodiment a unit dose of the composition comprises 100-500 mg SNAC, 5-50 mg PCSK9 inhibitor and 1-25 mg lubricant.

In a preferred embodiment a unit dose of the composition comprises 100-500 mg SNAC, 5-50 mg PCSK9 inhibitor and 1-25 mg lubricant.

In a preferred embodiment a unit dose of the composition comprises 80-1000 mg SNAC, 0.5-100 mg EGF(A) derivative and 1-50 mg lubricant.

In a preferred embodiment a unit dose of the composition comprises 100-800 mg SNAC, 20-120 mg EGF(A) derivative and 1-30 mg lubricant.

In a preferred embodiment a unit dose of the composition comprises 200-600 mg SNAC, 50-100 mg EGF(A) derivative and 1-20 mg lubricant.

In one embodiment the pharmaceutical composition of the invention has a fast disintegration or dissolution in vitro. Disintegration or dissolution may be tested as known in the art such as by using Assay II or Assay III described herein.

The dissolution or release may be expressed as the amount of the PCSK9 inhibitor measured in solution after a given period relative to the total content of the PCSK9 inhibitor of the composition. The relative amount may be given in percentage.

In one embodiment the release of the PCSK9 inhibitor from the pharmaceutical composition of the invention is at least 80% within 15 minutes or at least 95% within 30 minutes. In one such embodiment the release is measured at pH 6.8.

In one embodiment the pharmaceutical composition comprises
a) a PCSK9 inhibitor and
b) a salt of N-(8-(2-hydroxybenzoyl)amino)caprylic acid,
   wherein the release of the PCSK9 inhibitor reaches 80% within 15 minutes or 95% within 30 minutes. In one embodiment the release is measured at pH 6.8.

Experiments have demonstrated that PCSK9 inhibitor/SNAC composition according to the invention behave like semaglutide/SNAC composition with regards to disintegration and dissolution (Examples 2 and 3 herein). The improved plasma exposure of a PCSK9 inhibitor using a composition according to the invention compared to a PCSK9 inhibitor/SNAC composition prepared according to WO 2012/080471 and WO 2013/139694 similar to what has previously been observed for Semaglutide and other GLP-1 receptor agonists (PCT/EP2019/052487) has been demonstrated using Assay V herein (example 4).

In one embodiment the pharmaceutical composition of the invention provides an early exposure in vivo. In one embodiment the pharmaceutical composition of the invention provides an increased exposure in vivo. In one embodiment the pharmaceutical composition of the invention provides an increased early exposure in vivo. Such in vivo exposure may be tested in a relevant model, such as the Assay V described herein. The exposure may also be measured over a predetermined time period and the accumulative dose corrected AUC calculated, such as for t=0-30 minutes after dosing.

In one embodiment the invention relates to a pharmaceutical composition wherein the dose corrected plasma exposure at t=30 min after dosing is increased relative to a PCSK9 inhibitor composition prepared as described in WO2013/139694 substituting GLP-1 with a PCSK9 inhibitor. Alternatively, the reference may be test compositions 1 described herein.

In one embodiment the pharmaceutical composition comprises
a) a PCSK9 inhibitor and
b) a salt of N-(8-(2-hydroxybenzoyl)amino)caprylic acid,
   wherein the dose corrected plasma exposure at t=30 min after dosing is increased relative to a PCSK9 inhibitor composition prepared as type F of WO2013/139694.

In one embodiment the pharmaceutical composition comprises
a) a PCSK9 inhibitor and
b) a salt of N-(8-(2-hydroxybenzoyl)amino)caprylic acid,
   wherein the dose corrected AUC for t=0-30 min after dosing is increased relative to a PCSK9 inhibitor composition prepared as type F of WO2013/139694.

In one embodiment the dose corrected AUC for t=0-30 min after dosing is increased at least 1.2 fold, such as 1.5 fold, such as 2 fold compared to a PCSK9 inhibitor composition prepared as type F of WO2013/139694.

In one embodiment the pharmaceutical composition comprises
a) a PCSK9 inhibitor and
b) a salt of N-(8-(2-hydroxybenzoyl)amino)caprylic acid,
   wherein the dose corrected plasma exposure at t=30 min after dosing is increased relative Test composition 1 herein.

In one embodiment the pharmaceutical composition comprises
a) a PCSK9 inhibitor and
b) a salt of N-(8-(2-hydroxybenzoyl)amino)caprylic acid,
   wherein the dose corrected AUC for t=0-30 min after dosing is increased relative to Test composition 1 herein.

In one embodiment the dose corrected AUC for t=0-30 min is increased at least 1.2-fold, such as 1.5-fold, such as 2-fold compared relative to Test composition 1 herein.

### Dosage Form

The composition may be administered in several dosage forms, for example as a tablet; a coated tablet; a sachet or a capsule such as hard or soft shell gelatine capsules and all such compositions are considered solid oral dosage forms.

The composition may further be compounded in a drug carrier or drug delivery system, e.g. in order to improve stability and/or solubility or further improve bioavailability. The composition may be a freeze-dried or spray-dried composition.

The composition may be in the form of a dose unit, such as a tablet. In some embodiments the weight of the unit dose is in the range of 50 mg to 1000 mg, such as in the range of 50-750 mg, or such as in the range of 100-600 mg. In some embodiments the weight of the dose unit is in the range of 75 mg to 350 mg, such as in the range of 100-300 mg or such as in the range of 200-350 mg.

In some embodiments the weight of the dose unit is in the range of 100 mg to 400 mg, such as in the range of 50-300 mg or such as in the range of 200-400 mg.

In some embodiments the composition may be granulated prior to being compacted and i.e. compressed into tablets. The composition may comprise an intragranular part and/or an extragranular part, wherein the intragranular part has been granulated and the extragranular part has been added after granulation.

The intragranular part may comprise a PCSK9 inhibitor, the delivery agent and/or an excipient, such as a lubricant and/or glidant.

In some embodiments the intragranular part comprises the delivery agent and a lubricant and/or a glidant.

In some embodiments the extragranular part comprises the PCSK9 inhibitor, and/or a lubricant and/or a glidant, such as magnesium stearate. In some embodiments the extragranular part comprises the PCSK9 inhibitor. In some embodiments the extragranular part comprises an excipient, such as a lubricant and/or glidant, such as magnesium stearate.

In further embodiments the intragranular part comprises the PCSK9 inhibitor, the delivery agent and the lubricant and/or a glidant. In such embodiments the granulate may be directly compressed into tablets and the tablets have no extragranular part.

Preparation of Composition Preparation of a composition according to the invention may be performed according to methods known in the art.

To prepare a dry blend of tabletting material, the various components are optionally delumped or sieved, weighed, and then combined. The mixing of the components may be carried out until a homogeneous blend is obtained.

The terms “granulate” and “granules” are used interchangeably herein to refer to particles of composition material which may be prepared as described below. The term refers broadly to pharmaceutical ingredients in the form of particles, granules and aggregates which are used in the preparation of solid dose formulations. Generally, granules are obtained by processing a powder or a blend to obtain a solid which is subsequently used to obtain granules of the desired size.

If granules are to be used in the tabletting material, granules may be produced in a manner known to a person skilled in the art, for example using wet granulation methods known for the production of “built-up” granules or “broken-down” granules. Methods for the formation of built-up granules may operate continuously and comprise, for example simultaneously spraying the granulation mass with granulation solution and drying, for example in a drum granulator, in pan granulators, on disc granulators, in a fluidized bed, by spray-drying, spray-granulation or spray-solidifying, or operate discontinuously, for example in a fluidized bed, in a rotary fluid bed, in a batch mixer, such as a high shear mixer or a low shear mixer, or in a spray-drying drum. Methods for the production of broken-down granules, which may be carried out continuously or discontinuously and in which the granulation mass first forms a wet aggregate with the granulation solution, which is subsequently comminuted or by other means formed into granules of the desired size and the granules may then be dried. Suitable equipment for the wet granulation step are planetary mixers, low shear mixers, high shear mixers, extruders and spheronizers, such as an apparatus, but not limited to, from the companies Loedige, Glatt, Diosna, Fielder, Collette, Aeschbach, Alexanderwerk, Ytron, Wyss & Probst, Werner & Pfleiderer, HKD, Loser, Fuji, Nica, Caleva and Gabler. Granules may also be formed by dry granulation techniques in which one or more of the excipient(s) and/or the active pharmaceutical ingredient is compressed to form relatively large moldings, for example slugs or ribbons, which are comminuted by grinding, and the ground material serves as the tabletting material to be later compacted. Suitable equipment for dry granulation is, but not limited to, roller compaction equipment from Gerteis such as Gerteis MICRO-PACTOR, MINI-PACTOR and MACRO-PACTOR.

To compact the tabletting material into a solid oral dosage form, for example a tablet, a tablet press may be used. In a tablet press, the tabletting material is filled (e.g. force feeding or gravity feeding) into a die cavity. The tabletting material is then compacted by a set of punches applying pressure. Subsequently, the resulting compact, or tablet is ejected from the tablet press. The above-mentioned tabletting process is subsequently referred to herein as the “compaction process”. Suitable tablet presses include, but are not limited to, rotary tablet presses and eccentric tablet presses. Examples of tablet presses include, but are not limited to, the Fette 102i (Fette GmbH), the Korsch XL100, the Korsch PH 106 rotary tablet press (Korsch AG, Germany), the Korsch EK-O eccentric tabletting press (Korsch AG, Germany) and the Manesty F-Press (Manesty Machines Ltd., United Kingdom).

In general, granulates may be prepared by wet, melt or dry granulation, preferably dry granulation. Granules comprising i, ii and/or iii may thus be obtained by dry granulation of a blend hereof, such as by roller compaction. In an alternative embodiment wet granulation may be used to obtain the granules. This material can then be used directly or further refined to obtain the final granules.

In one embodiment the composition comprises at least one granulate. In one embodiment the composition comprises one type of granulate. The composition may alternatively comprise two types of granulates.

In an embodiment the invention relates to a composition comprising
a) a PCSK9 inhibitor,
b) a salt of N-(8-(2-hydroxybenzoyl)amino)caprylic acid (NAC) and
c) a lubricant
   wherein the composition comprises a granulate of b) and optionally c).

In further embodiments the granular part may comprise of a) and b) and optionally c).

The granulation maybe be obtained by various methods as described above, wherein a), b) and/or c) are initially mixed either as powders or by preparation of a solution comprising the one or more ingredients.

In some embodiments the method of preparation of the tablet comprises; a) granulating a mixture comprising the delivery agent and optionally a lubricant; b) blending the granulate of a) with a PCSK9 inhibitor and optionally additional lubricant, and then c) compressing the blend of b) into tablets.

In some embodiments the method of preparation of the tablet comprises; a) granulating a mixture comprising the delivery agent, the PCSK9 inhibitor and/or optionally a lubricant and b) compressing the granulate of a) into tablets and optionally including additional lubricant.

To obtain a homogenous granulate one or more sieving step(s) can be included prior to the final dry granulation step/roller compaction or tablet compression step.

Finally, additional excipient(s), such as a lubricant may be added prior to tablet compression forming an extragranular part.

### Pharmaceutical Indications

In one aspect the invention relates to the use of an PCSK9 inhibitor, such as an EGF(A) peptide analogue or an EGF(A) derivative for use in the manufacture of a pharmaceutical composition as described herein.

In one aspect the invention relates to a composition comprising a PCSK9 inhibitor, such as an EGF(A) peptide analogue or an EGF(A) derivative, for use as a medicament and/or in a method of treatment.

In one embodiment the composition is for use in a method of treatment, such as for (i) improving lipid parameters, such as prevention and/or treatment of dyslipidaemia, lowering total serum lipids; lowering LDL-C, increasing HDL; lowering small, dense LDL; lowering VLDL; lowering triglycerides; lowering cholesterol; lowering plasma levels of lipoprotein a (Lp(a)); inhibiting generation of apolipoprotein A (apo(A)); (

ii) the prevention and/or the treatment of cardiovascular diseases, such as cardiac syndrome X, atherosclerosis, myocardial infarction, coronary heart disease, reperfusion injury, stroke, cerebral ischemia, an early cardiac or early cardiovascular disease, left ventricular hypertrophy, coronary artery disease, hypertension, essential hypertension, acute hypertensive emergency, cardiomyopathy, heart insufficiency, exercise intolerance, acute and/or chronic heart failure, arrhythmia, cardiac dysrhythmia, syncopy, angina pectoris, cardiac bypass and/or stent reocclusion, intermittent claudication (atheroschlerosis oblitterens), diastolic dysfunction, and/or systolic dysfunction; and/or the reduction of blood pressure, such as reduction of systolic blood pressure; the treatment of cardiovascular disease.

Dyslipidaemia may be such as a high plasm concentration of cholesterols also called hypercholesterolaemia referring to a situation where the plasma cholesterol concentrations is above the normal range of a total cholesterol 5.0 mmol/l. In one embodiment the compound or composition of the invention may be used for treatment of hypercholesterolaemia.

### Method of Treatment

The invention further relates to a method of treating a subject in need thereof, comprising administering a therapeutically effective amount of a composition according to the present invention to said subject. In one embodiment the method of treatment is for (i) improving lipid parameters and/or (ii) preventing and/or treating cardiovascular diseases and/or the further indications specified above.

In some embodiments, a method is described comprising administering to a subject in need thereof a therapeutically effective amount of a pharmaceutical composition comprising a PCSK9 inhibitor, a salt of N-(8-(2-hydroxybenzoyl)amino)caprylic acid (NAC), and optionally, a lubricant.

In some embodiments, a method for treating diabetes is described comprising administering to a subject in need thereof a therapeutically effective amount of a pharmaceutical composition comprising
a) a PCSK9 inhibitor,
b) a salt of N-(8-(2-hydroxybenzoyl)amino)caprylic acid (NAC) and
c) a lubricant as described herein above.

Various examples of a lubricant are described, including magnesium stearate. The composition is administered orally and is in a form of a table, capsule or a sachet.

In a further such embodiments one or more dose units may be administered to said subject in need.

### Combination Treatment

Treatment with a PCSK9 inhibitor according to the present invention may be combined with treatment with one or more additional pharmacologically active substances, e.g. selected from anti-diabetic agents, anti-obesity agents, appetite regulating agents, antihypertensive agents, agents for the treatment and/or prevention of complications resulting from or associated with diabetes and agents for the treatment and/or prevention of complications and disorders resulting from or associated with obesity.

Examples of such pharmacologically active substances are: GLP-1 receptor agonists, insulin, DPP-IV (dipeptidyl peptidase-IV) inhibitors, amylin agonists and leptin receptor agonists. Particular examples of such active substances are the GLP-1 receptor agonists liraglutide and semaglutide and insulin degludec.

The invention as described herein is, without limitation hereto, further defined by the embodiments described here below and the claims of the document.

### EMBODIMENTS

1. A pharmaceutical composition comprising
   a) a PCSK9 inhibitor and
   b) a salt of N-(8-(2-hydroxybenzoyl)amino)caprylic acid
      wherein said salt of N-(8-(2-hydroxybenzoyl)amino)caprylic acid constitutes at least 60 w/w % of the composition.
2. A pharmaceutical composition comprising
   a) a PCSK9 inhibitor and
   b) a salt of N-(8-(2-hydroxybenzoyl)amino)caprylic acid
      wherein said salt of N-(8-(2-hydroxybenzoyl)amino)caprylic acid constitutes at least 90 w/w %, such as at least 95 w/w % of the excipients of the composition.
3. A pharmaceutical composition consisting of
   a) a PCSK9 inhibitor and
   b) excipients, wherein the excipients are
      i. a salt of N-(8-(2-hydroxybenzoyl)amino)caprylic acid and
      ii. one or more further excipients
         wherein said salt of N-(8-(2-hydroxybenzoyl)amino)caprylic acid constitutes at least 90 w/w %, such as at least 95 w/w % of the excipients of the composition.
4. The pharmaceutical composition according to any of the previous embodiments 1-3, wherein the composition comprises at least one lubricant.
5. A pharmaceutical composition consisting of:
   a) a PCSK9 inhibitor,
   b) a salt of N-(8-(2-hydroxybenzoyl)amino)caprylic acid and
   c) at least one lubricant
6. The pharmaceutical composition according to any of the previous embodiments 4 and 5, wherein the lubricant is magnesium stearate.
7. The pharmaceutical composition according any of the previous embodiments, wherein the composition comprises 1-25 mg, such as 1-10 mg, such as 2-5 mg or such as 2-3 mg magnesium stearate per 100 mg salt of N-(8-(2-hydroxybenzoyl)amino)caprylic acid.
8. The pharmaceutical composition according to any of the previous embodiments 2-7, wherein said salt of N-(8-(2-hydroxybenzoyl)amino)caprylic acid constitutes at least 60 w/w % of the composition.
9. The pharmaceutical composition according to any of the previous embodiments 1, 5-8, wherein said salt of N-(8-(2-hydroxybenzoyl)amino)caprylic acid constitutes at least 95 w/w % of the excipients of the composition.
10. The pharmaceutical composition according to any of the previous embodiments, wherein the salt of N-(8-(2-hydroxybenzoyl)amino)caprylic acid is selected from the group consisting of the sodium salt, potassium salt of N-(8-(2-hydroxybenzoyl)amino)caprylic acid.
11. The pharmaceutical composition according to any of the previous embodiments, wherein the salt of N-(8-(2-hydroxybenzoyl)amino)caprylic acid is sodium N-(8-(2-hydroxybenzoyl)amino)caprylate (SNAC).
12. The pharmaceutical composition according to any of the previous embodiments, wherein a dose unit comprises at most 1000 mg of said salt of N-(8-(2-hydroxybenzoyl)amino)caprylic acid.
13. The pharmaceutical composition according to any of the previous embodiments, wherein a dose unit comprises 0.1-100 mg of the PCSK9 inhibitor, such as 1-100 mg of the PCSK9 inhibitor.
14. The pharmaceutical composition according to any of the previous embodiments, wherein the PCSK9 inhibitor has an inhibitory function at least comparable to EGF(A) 301L.
15. The pharmaceutical composition according to any of the previous embodiments, wherein the apparent binding affinity (Ki) for the PCSK9 inhibitor is equal or below the apparent binding affinity (Ki) for EGF(A) 301L.
16. The pharmaceutical composition according to any of the previous embodiments, wherein the PCSK9 inhibitor has an inhibitory function at least comparable to EGF(A) 301L,309R,312E.
17. The pharmaceutical composition according to any of the previous embodiments, wherein the apparent binding affinity (Ki) for the PCSK9 inhibitor is equal to or below the apparent binding affinity (Ki) for EGF(A) 301L,309R,312E.
18. The pharmaceutical composition according to any of the previous embodiments, wherein

$$\frac{Ki ( PCSK 9 inhibitor )}{Ki ( EG F ( A ) 301 L )}$$

is below 2.

19. The pharmaceutical composition according to any of the previous embodiments, wherein

$$\frac{Ki ( PCSK 9 inhibitor )}{Ki ( EG F ( A ) 301 L , 3 \; 09 R , 312 E )}$$

is below 2.
20. The pharmaceutical composition according to any of the previous embodiments, wherein the PCSK9 inhibitor has an apparent binding affinity (Ki) below 10 nM, such as below 8 nM, such as below 5 mM.
21. The pharmaceutical composition according to any of the previous embodiments 14-20, wherein the apparent binding affinity (Ki) is measured in a competitive ELISA as described in Assay I.
22. The pharmaceutical composition according to any of the previous embodiments, wherein the PCSK9 inhibitor has T ½ of at least 24 hours in mini pigs
23. The composition according to any of the previous embodiments, wherein the PCSK9 inhibitor has T ½ of at least 2 hours in rats.
24. The composition according to any of the previous embodiments, wherein the PCSK9 inhibitor has a molar mass of at most 50000 g/mol.
25. The composition according to any of the previous embodiments, wherein the PCSK9 inhibitor is an EGF(A) peptide or an EGF(A) derivative.
26. The composition according to any of the previous embodiments, wherein the EGF(A) derivative according to embodiment 24 comprises an albumin binding substituent.
27. The composition according to any of the previous embodiments, wherein the EGF(A) derivative according to embodiment 24 or 25 comprises a fatty acid or a fatty diacid.
28. The composition according to any of the previous embodiments, wherein the EGF(A) derivative according to embodiment 24, 25 or 26 comprises a C16, C18 or C20 fatty acid or a C16, C18 or C20 fatty diacid.
29. The composition according to any of the embodiments 24-27, wherein the EGF(A) peptide or EGF(A) derivative comprises an EGF(A) peptide analogue having 1-8 amino acid substitutions compared to the EGF(A) domain of LDL-R defined by SEQ ID NO 1.
30. The composition according to embodiment 28, wherein the EGF(A) peptide analogue comprises 301Leu.
31. The pharmaceutical composition according to any of the previous embodiments, wherein the PCSK9 inhibitor is selected from the group consisting of: EGF(A) derivatives 31, 95, 128, 133, 143, 144, 150, 151, 152 and 153 shown below

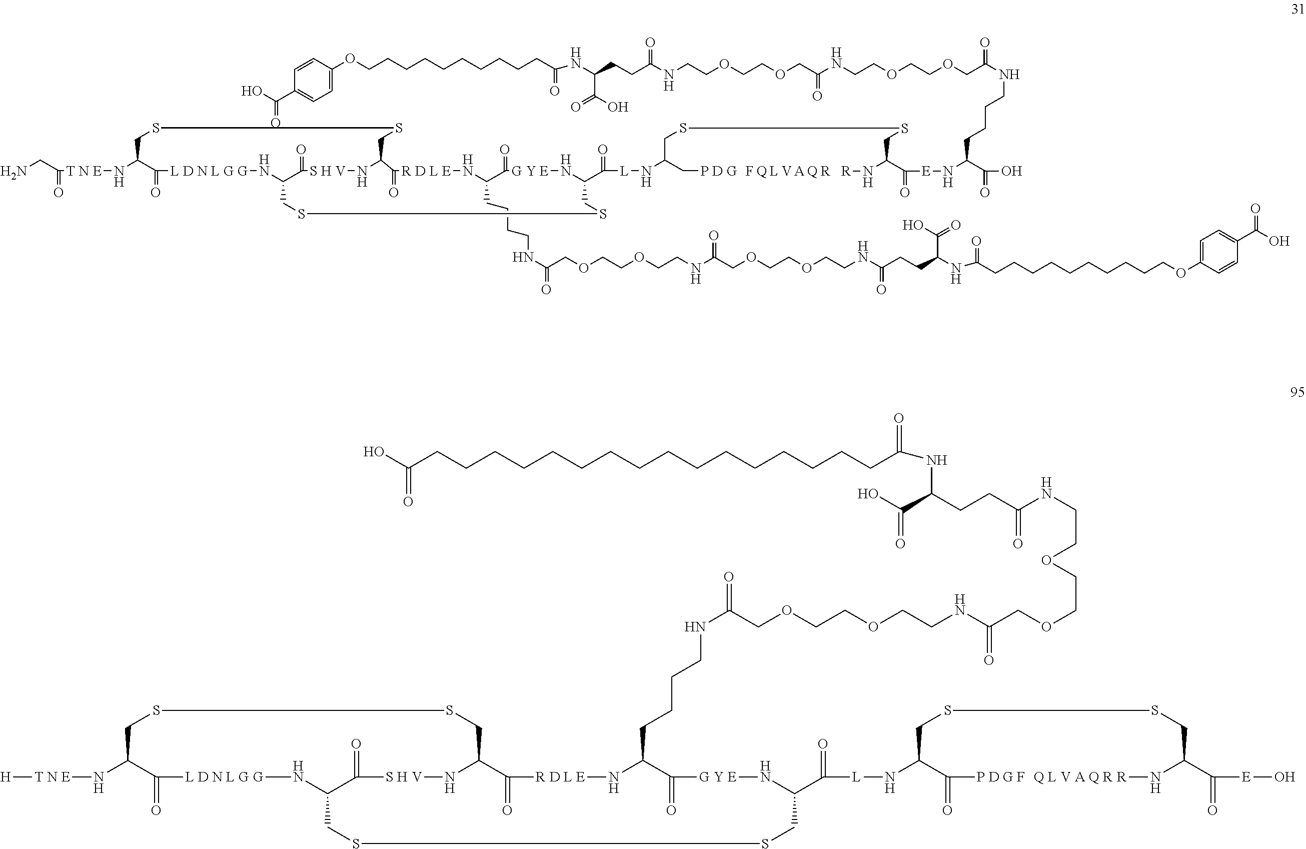

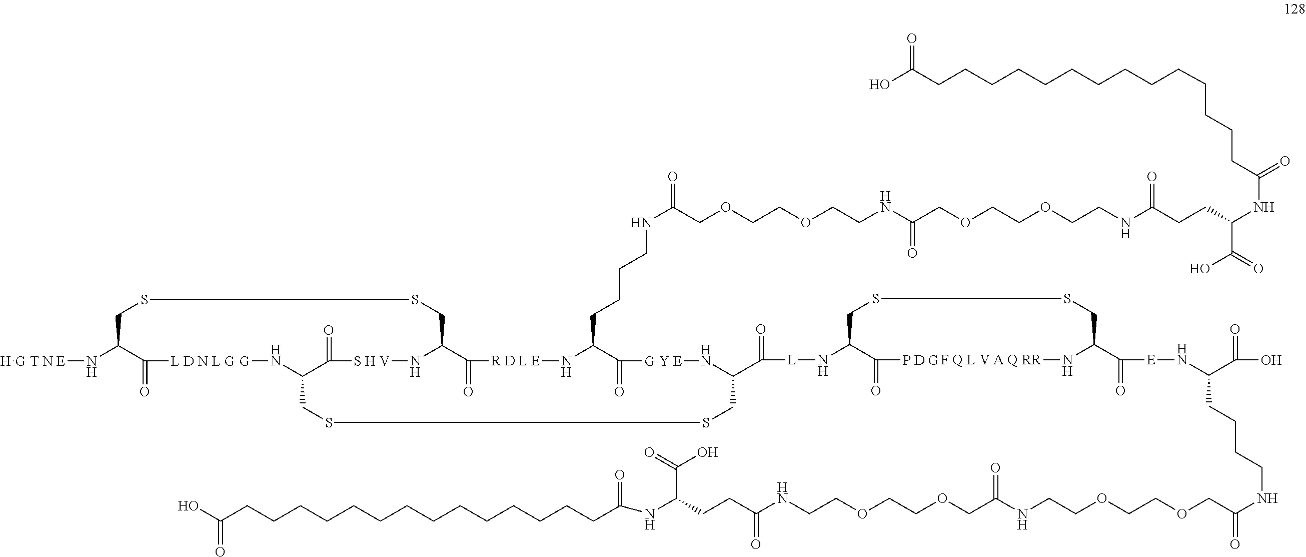

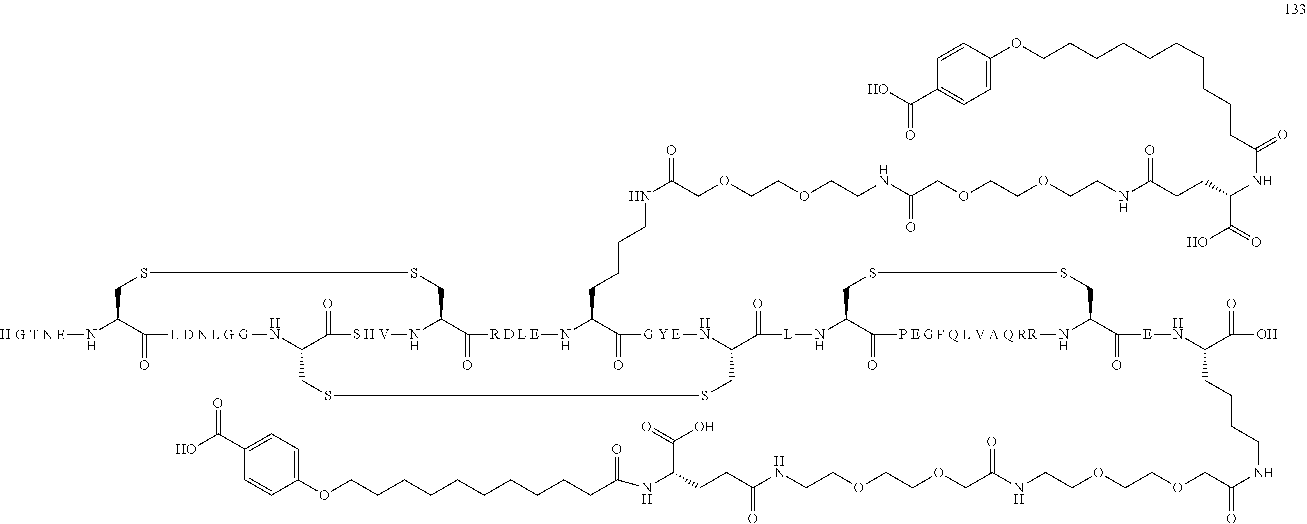

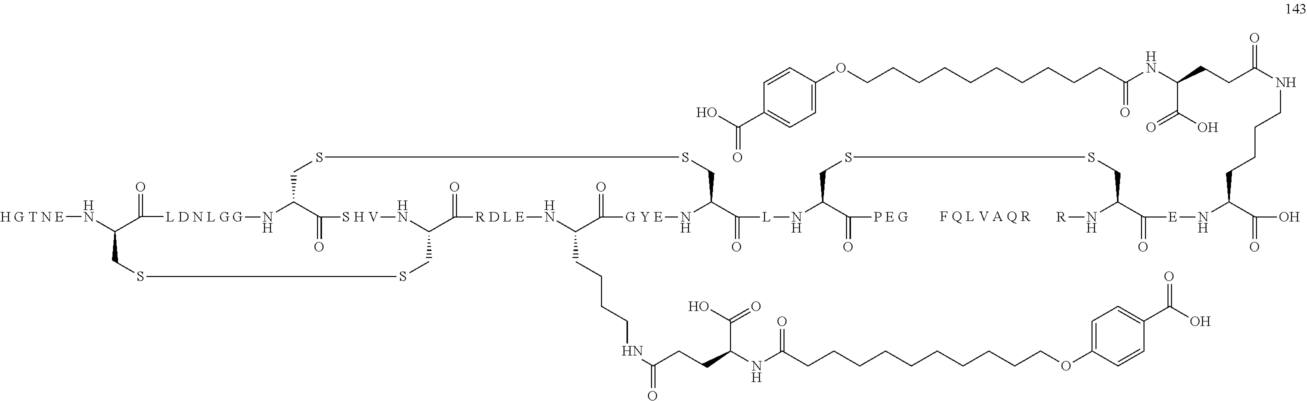

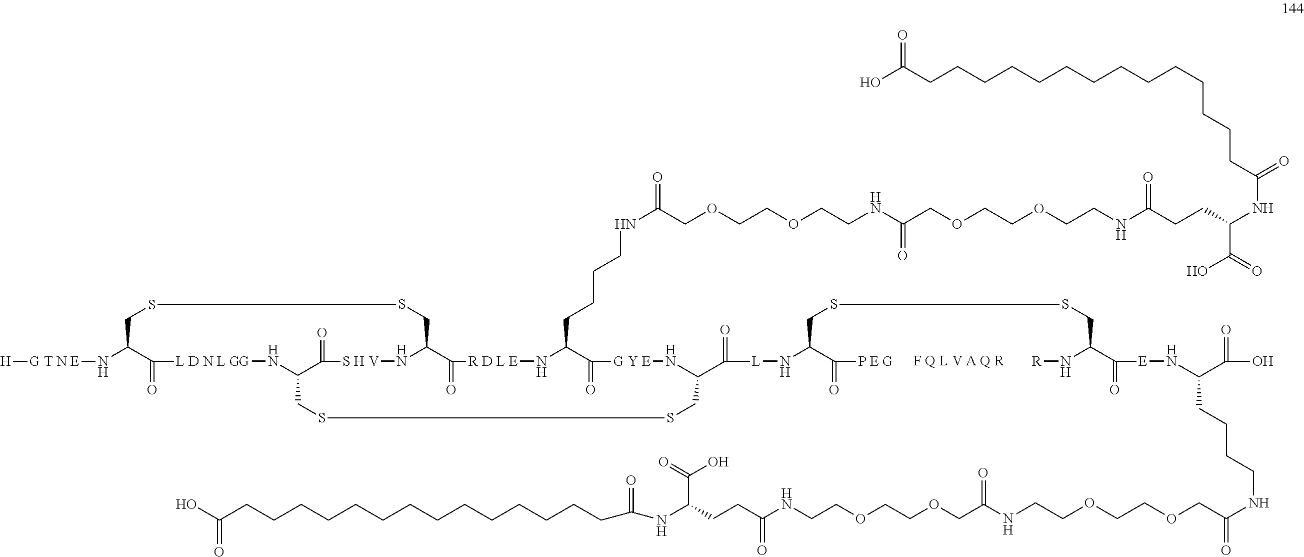

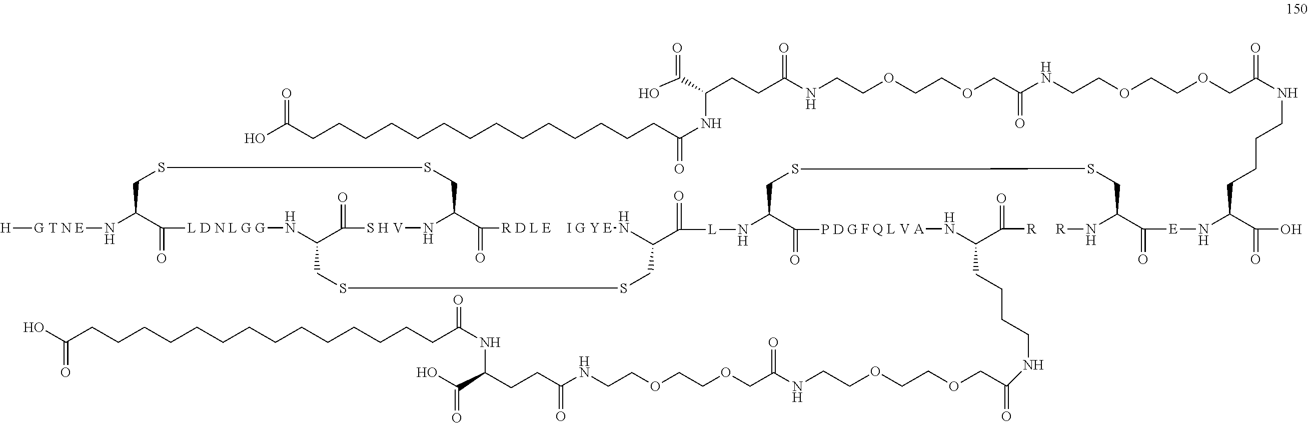

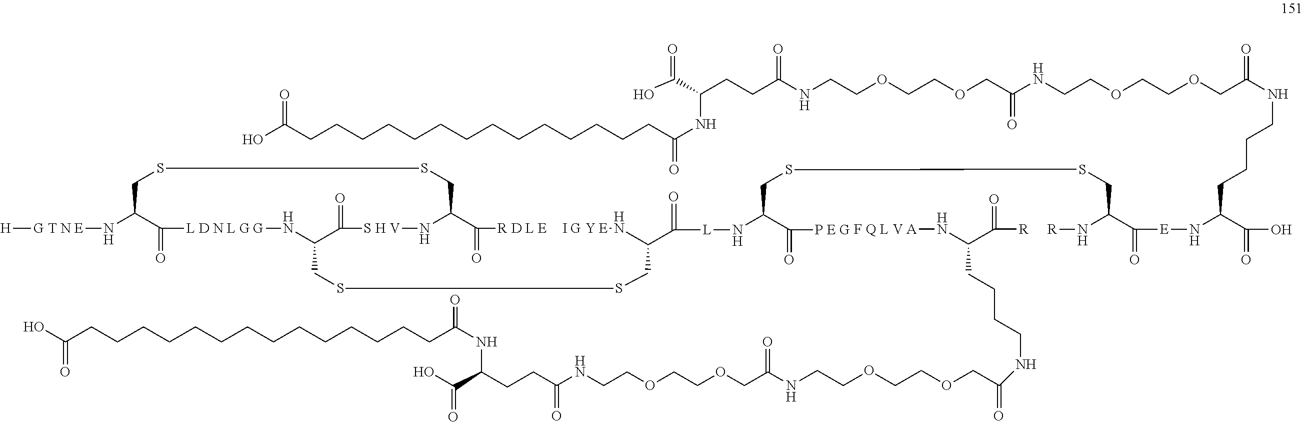

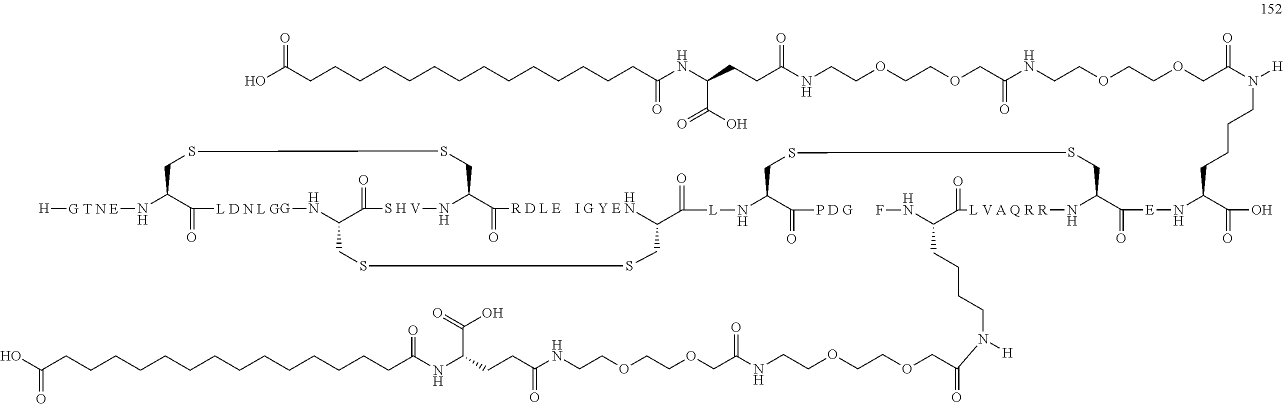

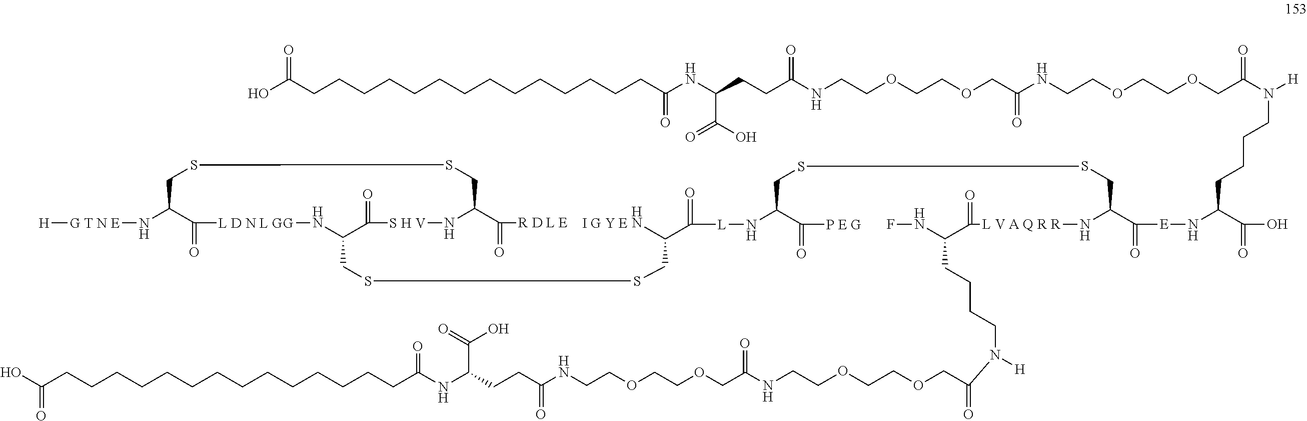

32. The pharmaceutical composition according to any of the previous embodiments, wherein the PCSK9 inhibitor is selected from the group consisting of EGF(A) derivative 150, 151, 152 and 153.
33. The pharmaceutical composition according to any of the previous embodiments, wherein the PCSK9 inhibitor is:

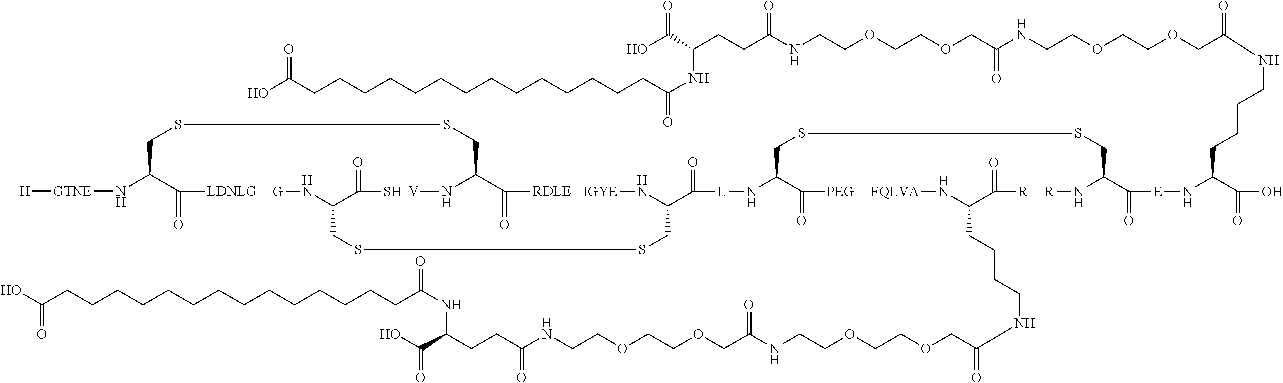

34. The pharmaceutical composition according to any of the previous embodiments, wherein the composition comprises at least one granulate.
35. The pharmaceutical composition according to previous embodiment 34, wherein the at least one granulate comprises the salt of N-(8-(2-hydroxybenzoyl)amino)caprylic acid.
36. The pharmaceutical composition according to any of the previous embodiments 34-35, wherein the at least one granulate further comprises a lubricant, such as magnesium stearate.
37. The pharmaceutical composition according to any of the previous embodiments 34-36, wherein the at least one granulate further comprises the PCSK9 inhibitor.
38. The pharmaceutical composition according to any of the previous embodiment 34-37, wherein the at least one granulate is prepared by dry granulation, such as by roller compaction.
39. The pharmaceutical composition according to any of the previous embodiment 34-38, wherein the composition comprises an extra-granular part.
40. The pharmaceutical composition according to any of the previous embodiment 34-39, wherein the extra-granular part of the composition comprises a lubricant or glidant, such as magnesium stearate and/or the PCSK9 inhibitor.
41. A pharmaceutical composition comprising
   a) 0.1-100 mg of a PCSK9 inhibitor and
   b) 20-800 mg, such as 25-700, such as 50-600 mg of a salt of N-(8-(2-hydroxybenzoyl)amino)caprylic acid,
      wherein said salt of N-(8-(2-hydroxybenzoyl)amino)caprylic acid constitutes at least 90 w/w %, such as at least 95 w/w % of the excipients of the composition and wherein the PCSK9 inhibitor is the EGF(A) derivative shown as Examples 151 in WO2017/121850.
42. A pharmaceutical composition comprising
   a) 1-100 mg of a PCSK9 inhibitor and
   b) 50-800 mg of a salt of N-(8-(2-hydroxybenzoyl)amino)caprylic acid, wherein said salt of N-(8-(2-hydroxybenzoyl)amino)caprylic acid constitutes at least 90 w/w %, such as at least 95 w/w % of the excipients of the composition and wherein the PCSK9 inhibitor is the EGF(A) derivative shown as Examples 151 in WO2017/121850.
43. A pharmaceutical composition comprising
   a) 1-100 mg of a PCSK9 inhibitor and
   b) 75-600 mg of a salt of N-(8-(2-hydroxybenzoyl)amino)caprylic acid, wherein said salt of N-(8-(2-hydroxybenzoyl)amino)caprylic acid constitutes at least 90 w/w %, such as at least 95 w/w % of the excipients of the composition and wherein the PCSK9 inhibitor is the EGF(A) derivative shown as Examples 151 in WO2017/121850.
44. A pharmaceutical composition comprising
   a) 1-100 mg of a PCSK9 inhibitor and
   b) 75-400 mg of a salt of N-(8-(2-hydroxybenzoyl)amino)caprylic acid, wherein said salt of N-(8-(2-hydroxybenzoyl)amino)caprylic acid constitutes at least 90 w/w %, such as at least 95 w/w % of the excipients of the composition and wherein the PCSK9 inhibitor is the EGF(A) derivative shown as Examples 151 in WO2017/121850.
45. A pharmaceutical composition comprising
   a) 1-100 mg of a PCSK9 inhibitor and
   b) 100-400 mg of a salt of N-(8-(2-hydroxybenzoyl)amino)caprylic acid, wherein said salt of N-(8-(2-hydroxybenzoyl)amino)caprylic acid constitutes at least 90 w/w %, such as at least 95 w/w % of the excipients of the composition and wherein the PCSK9 inhibitor is the EGF(A) derivative shown as Examples 151 in WO2017/121850.
46. A pharmaceutical composition comprising
   a) 1-100 mg of a PCSK9 inhibitor and
   b) 200-600 mg of a salt of N-(8-(2-hydroxybenzoyl)amino)caprylic acid, wherein said salt of N-(8-(2-hydroxybenzoyl)amino)caprylic acid constitutes at least 90 w/w %, such as at least 95 w/w % of the excipients of the composition and wherein the PCSK9 inhibitor is the EGF(A) derivative shown as Examples 151 in WO2017/121850.
47. A pharmaceutical composition comprising
   a) 5-100 mg of a PCSK9 inhibitor and
   b) 250-500 mg of a salt of N-(8-(2-hydroxybenzoyl)amino)caprylic acid, wherein said salt of N-(8-(2-hydroxybenzoyl)amino)caprylic acid constitutes at least 90 w/w %, such as at least 95 w/w % of the excipients of the composition and wherein the PCSK9 inhibitor is the EGF(A) derivative shown as Examples 151 in WO2017/121850.
48. The pharmaceutical composition according to any of the embodiments 41-47, further comprising 1-26 mg lubricant, such as magnesium stearate.
49. The pharmaceutical composition according to any of the embodiments 41-47, further comprising 1-25 mg, such as 1-10 mg, such as 2-5 mg or such as 2-3 mg magnesium stearate per 100 mg salt of N-(8-(2-hydroxybenzoyl)amino)caprylic acid.
50. The pharmaceutical composition according to any of the embodiments 41-47, wherein the salt of N-(8-(2-hydroxybenzoyl)amino)caprylic acid is sodium N-(8-(2-hydroxybenzoyl)amino)caprylate (SNAC).
51. The pharmaceutical composition according to any of the previous embodiment, wherein the composition is for oral administration.
52. The pharmaceutical composition according to any of the previous embodiments, wherein the composition is a solid composition.
53. The pharmaceutical composition according to the previous embodiments, wherein the composition is a solid composition, such as a tablet, a capsule or a sachet.
54. A pharmaceutical composition comprising
   a) a PCSK9 inhibitor and
   b) a salt of N-(8-(2-hydroxybenzoyl)amino)caprylic acid, wherein the release of the PCSK9 inhibitor reaches 80% within 15 minutes or 95 within 30 minutes.
55. A pharmaceutical composition comprising
   a) a PCSK9 inhibitor and
   b) a salt of N-(8-(2-hydroxybenzoyl)amino)caprylic acid, wherein the dose corrected plasma exposure at t=30 min after dosing is increased relative to a test composition 1.
56. A pharmaceutical composition comprising
   a) a PCSK9 inhibitor and
   b) a salt of N-(8-(2-hydroxybenzoyl)amino)caprylic acid,
      wherein the dose corrected AUC for t=0-30 min after dosing is increased relative to test composition 1.
57. The pharmaceutical composition according to any of the previous embodiments 1-53, wherein
   a) the release of the PCSK9 inhibitor reaches 80% within 15 minutes
   b) the release of the PCSK9 inhibitor reaches 95% within 30 minutes
   c) the dose corrected plasma exposure at t=30 min after dosing is increased relative to test composition 1 herein and/or
   d) the dose corrected AUC for t=0-30 min after dosing is increased relative to test composition 1.
58. The pharmaceutical composition according to embodiment 54 or embodiment 57, wherein the dose corrected AUC for T=0-30 min is increased at least 1.2 fold, such as 1.5 fold, such as at least 2 fold.
59. The pharmaceutical composition according to any of the embodiment 54-58, wherein the release is determined as in Assay Ill herein and/or the dose corrected plasma exposures is determined as in Assay V.
60. The pharmaceutical composition according to any of the embodiments 41-50 further defined by the features of one or more of the embodiments 1-13 and 34-40.
61. The pharmaceutical composition according to any of the embodiments 53-59 further defined by the features of one or more of the embodiments 1-52.
62. A pharmaceutical composition according to any of the previous embodiments for use in medicine.
63. A pharmaceutical composition according to any of the previous embodiments for use in a method of i) improving lipid parameters and/or (ii) preventing and/or treating cardiovascular diseases.
64. A method of treatment of a subject in need thereof, wherein the method comprises administering a therapeutically active amount of a composition according to any of the previous embodiments to said subject.

### METHODS AND EXAMPLES

### General Methods of Detection and Characterisation

Assay I: PCSK9-LDL-R binding—Competitive (ELISA)

This assay measures the apparent binding affinity to PCSK9 in competition with LDL-R. In particular the assay is used to evaluate the apparent binding affinity of an PCSK9 inhibitor such as an EGF(A) analogue and compounds comprising an EGF(A) analogue The assay is performed as follows. The day before the experiment, recombinant human Low Density Lipoprotein Receptor (rhLDL-R; NSO-derived; R & D systems # 2148-LD) is dissolved at 1 μg/ml in 50 mM sodium carbonate, pH 9.6, and then 100 μl of the solution is added to each well of the assay plates (Maxisorp 96, NUNC # 439454) and coated overnight at 4° C. On the day of the experiments, 8 point concentration curves of the EGF(A) compounds containing Biotinylated PCSK9 (0.5 ug/ml, BioSite/BPSBioscience cat#71304) are made in duplicate. Test compound and biotinylated PCSK9 mixtures are prepared and incubated for 1 hour at room temperature in assay buffer containing 25 mM Hepes, pH 7.2 (15630-056, 100 ml, 1M), 150 mM NaCl (Emsure 1.06404.1000) 1% HSA (Sigma A1887-25G) 0.05% Tween 20 (Calbiochem 655205) 2 mM CaCl₂ (Sigma 223506-500G). The coated assay plates are then washed 4× in 200 μl assay buffer, and then 100 pl of the mixture of test compounds and biotinylated PCSK9 is added to the plates and incubated 2 h at room temperature. The plates are washed 4× in 200 μl assay buffer and then incubated with Streptevadin-HRP (25ng/ml; VWR # 14-30-00) for 1 h at room temperature. The reaction is detected by adding 50 pl TMB-on (KEM-EN-TEC) and incubated 10 min in the dark. Then the reaction is stopped by adding 50 μl 4 M H₃PO₄ to the mixture, added by electronic multi pipetting. The plates are then read in a Spectramax at 450 and 620 nm within 1 h. The 620 nm read is used for background subtraction. 1050 values are calculated using Graphpad Prism, by nonlinear regression log(inhibitor) vs. response-variable slope (four parameters), and converted into Ki values using the following formula: Ki=IC50/(1+(Biotin-PCSK9)/(kd(Biotin-PCSK9))), where Kd of the biotin-PCSK9 is 1.096727714 μg/ml and [Biotin-PCSK9] =0.5 μg/ml.

Higher Ki values reflects lower apparent binding affinities to PCSK9 and vice versa. A value above 500 nM, will indicate that the observed binding is not specific.

Ki values for examples of EGF(A) peptide and derivatives thereof are included below, showing that the high affinity of compounds having an EGF(A) peptide including 301L and optionally one or more of 309R, 312E and 321E is very similar also including compounds with one or two substituents attached to the N-terminal or a Lysine residue.

| | Ki |
|---|---|
| EGF(A) peptide | (nM) |
| EGF(A) LDL R (293 332) | — |
| 299A, 301L, 3071, 309R, 310K | 9.4 |
| 299A, 301L, 3071, 309R | 0.9 |
| 301L, 309R, 310K | 7.3 |
| 301L, 309R | 1.2 |
| 301L | 2.8 |
| 301L, 309R, 312E | 1.1 |

EGF(A) peptide derivatives

| | | SEQ | | | |
|---|---|---|---|---|---|
| Example | | ID | | Attachment | Ki |
| compound # | EGF(A) peptide | NO | Substituent | site(s) | (nM) |
| 3 | 301L, 309R, 312E, | 4 | HOOC—(CH₂)₁₆—CO-gGlu-2xADO | 333K | 0.8 |
| | 333K | | | | |
| 8 | 301L, 309R, 312E | 6 | HOS(O)₂—(CH2)1₅—CO-gGlu- | N-term | 1.2 |
| | | | 2xADO—NH—CH₂—(C₆H₄)—CH2— | | |
| 31 | 301L, 309R, 312E, | 32 | 4-HOOC—(C₆H₄)—O—(CH₂)₁₀— | 313K, 333K | 0.5 |
| | 313K, 333K | | CO-gGlu-2xADO | | |
| 95 | des293, 301L, | 76 | HOOC—(CH₂)i6—CO-gGlu-2xADO | 313K | 1.5 |
| | 309R, 312E, 313K | | | | |
| 128 | 301L, 309R, 312E, | 32 | HOOC—(CH₂)i₄—CO-gGlu-2xADO | 313K, 333K | 1.0 |
| | 313K, 333K | | | | |
| 133 | 301L, 309R, 312E, | 98 | 4-HOOC—(C₆H₄)—O—(CH₂)₁₀— | 313K, 333K | 1.6 |
| | 313K, 321E, 333K | | CO-gGlu-2xADO | | |
| 143 | 301L, 309R, 312E, | 98 | 4-HOOC—(C₆H₄)—P—(CH₂)₁₀—CO-gGlu | 313K, 333K | 2.0 |
| | 313K, 321E, 333K | | | | |
| 144 | 301L, 309R, 312E, | 98 | HOOC—(CH₂)i₄—CO-gGlu-2xADO | 313K, 333K | 2.09 |
| | 313K, 321E, 333K | | | | |
| 150 | 301L, 309R, 312E, | 78 | HOOC—(CH₂)i₄—CO-gGlu-2xADO | 328K, 333K | 2.3 |
| | 328K, 333K | | | | |
| 151 | 301L, 309R, 312E, | 104 | HOOC—(CH2)14—CO-gGlu-2xADO | 328K, 333K | 1.8 |
| | 321E, 328K, 333K | | | | |
| 152 | 301L, 309R, 312E, | 72 | HOOC—(CH2)14—CO-gGlu-2xADO | 324K, 333K | 1.9 |
| | 324K, 333K | | | | |
| 153 | 312E, 321E, 324K, | 105 | HOOC—(CH₂)i4—CO-gGlu-2xADO | 324K, 333K | 2.0 |
| | 333K | | | | |

### Assay II: Disintegration Test

A standard disintegration test according to the European Pharmacopeia (Ph Eur 2.9.1) may be performed in an appropriate disintegration apparatus e.g. USP disintegration apparatus to measure the disintegration time of the test compositions in vitro.

### Assay III: Dissolution Test

A standard dissolution test according to the European Pharmacopeia (Ph Eur 2.9.3) may be performed to measure the release of the PCSK9 inhibitor and SNAC from the test compositions in vitro.

A dissolution test is performed in an appropriate dissolution apparatus e.g. USP dissolution apparatus 2. More specifically, an apparatus 2 is used in accordance with United States Pharmacopoeia 35 using a paddle rotation speed of 50 rpm. For testing at pH 6.8, the 500 mL dissolution medium of 0.05 M phosphate buffer is used at a temperature of 37±0.5° C. Dissolution media has a content of 0.1% Brij®35. Samples are removed at appropriate intervals and sample content is determined using a RP-UHPLC method for dual detection of PCSK9 inhibitor and SNAC.

The sample content is calculated based on the peak area of the PCSK9 inhibitor and SNAC in the chromatogram relative to the peak areas of the PCSK9 inhibitor and SNAC references, respectively. The released amount of PCSK9 inhibitor and SNAC is calculated as percentages of the nominal or actual total content in the test compositions. The total content in the tablets is determined using Assay (IV).

### Assay IV: Analysis of Amount of PCSK9 Inhibitor and SNAC

For assay analysis the test compositions are weighed before extraction of the PCSK9 inhibitor and SNAC. Tablets are dissolved in a relevant amount of 0.05 M phosphate buffer, pH 7.4, with 20% acetonitrile. Extraction time of two hours is used. Samples are centrifuged, and a suitable volume is transferred to a HPLC vial. Standards of relevant PCSK9 inhibitor and SNAC are prepared by using the same diluent as for the samples. UHPLC with an UV-detector is used for dual determination of the PCSK9 inhibitor and SNAC content. The tablet content is calculated based on the peak area of the PCSK9 inhibitor and SNAC in the chromatogram relative to the peak areas of the PCSK9 inhibitor and SNAC and references, respectively.

### Assay V: Pharmacokinetic Studies in Beagle Dogs

Pharmacokinetic (PK) studies in Beagle dogs are conducted to determine the exposure of the PCSK9 inhibitor after peroral administration of different test compositions.

For the pharmacokinetic studies male Beagle dogs are used, 1 to 6 years of age and weighing approximately 10-16 kg at the start of the studies. The dogs are group housed in pens (12 hours light: 12 hours dark) and fed individually and restrictedly once daily with adult dog diet (Royal Canine), Denmark). Exercise and group socialising are permitted daily, whenever possible. The dogs are used for repeated pharmacokinetic studies with a suitable wash-out period between successive dosing. An appropriate acclimatisation period is given prior to initiation of the first pharmacokinetic study. All handling, dosing and blood sampling of the animals are performed by trained and skilled staff. Before the studies the dogs are fasted overnight and from 0 to 4 h after dosing. Besides, the dogs are restricted to water 1 hour before dosing until 4 hours after dosing, but otherwise have ad libitum access to water during the whole period.

The tablets containing the PCSK9 inhibitor are administered in the following manner: 10 min prior to tablet administration the dogs are dosed subcutaneously with approximately 3 nmol/kg of SEQ ID NO: 115. The PCSK9 inhibitor tablets are placed in the back of the mouth of the dog to prevent chewing (one tablet/dog). The mouth is then closed and tap water is given by a syringe or gavage to facilitate swallowing of the tablet.

### Blood Sampling

Blood is sampled at predefined time points for up till 10 hr post dosing to adequately cover the full plasma concentration-time absorption profile of the PCSK9 inhibitor. For each blood sampling time point approximately 1.2 mL of whole blood is collected in a 1.3 mL EDTA coated tube, and the tube is gently turned to allow mixing of the sample with the EDTA. Then, the blood samples are kept on ice until centrifugation (4 min, 4° C., 4000 rpm) for plasma collection. Plasma (200 μl) is pipetted into Micronic tubes on dry ice and kept at −20° C. until analysis.

Blood samples are taken as appropriate, for example from a venflon in the cephalic vein in the front leg for the first 2 hours and then with syringe from the jugular vein for the rest of the time points (the first few drops are allowed to drain from the venflon to avoid heparin saline from the venflon in the sample).

### Assay VI: Bioanalytical Assays

Plasma concentrations of PCSK9 inhibitor from the oral and i.v. PK studies in dogs were analysed by following assay: plasma samples were incubated with guanidine after crashed by protein precipitation and analysed by turboflow liquid chromatography mass spectrometry (TF-LC-MS). Calibrators were prepared by spiking blank dog plasma with PCSK9 inhibitor, typically in the range from 0.5 to 2000 nM (LLOQ was 2 nM). Calibrators, plasma blanks or study samples were mixed 1:1 with 8M guanidine-hydrochloride (GndHCI) to a final concentration of 4M GndHCI and incubated for 30 minutes at 37° C. to dissociate non-covalent protein interactions. One volume of calibrator, plasma blank or study sample was precipitated with 2.5 volumes of ice cold methanol and centrifuged at 6200 rpm at 4° C. for 15 minutes. The supernatant was diluted with water +1% formic acid in ratio 1:2 following by TF-LC-MS analysis. Cyclone turboflow column (TurboFlow Cyclone 0,5×50 mm, Thermo Fischer Scientific), at room temperature and an Aeris Peptide 3.6 μm XB-C18 analytical column (2.1×50 mm, Phenomenex) at 60° C. were used. A gradient elution was conducted using mobile phase A (consisting of milli-Q water with 1% formic acid and 5% methanol/acetonitrile (50/50)) and mobile phase B (consisting of methanol/acetonitrile (50/50) with 1% formic acid and 5% milli-Q water). A QExactive Plus mass spectrometer was used as detector in single ion monitoring mode (m/z 1185-1188). Linear calibration curves (weighed 1/x2) were used for calculating the concentration in the plasma samples. Quality control samples were included. The deviation between nominal and calculated concentration in the calibrators and quality control samples was below 15%.

### General Methods

### Method 1: Dry Granulation

Dry granulation is carried out by roller compaction on a Gerteis MICRO-PACTOR or MINI-PACTOR. The roller speed is set at 1 or 3 rpm, roller compaction force at 6 kN/cm, and gap of 1 mm. Subsequent to dry granulation comminution of the mouldings into granules is carried out using a 0.63 mm or 0.8 mm screen.

Prior to dry granulation, SNAC and magnesium stearate and optionally EGF(A) derivativeand/or parts of MCC are blended in a suitable blender such as a Pharmatech V-shell blender or Turbula mixer.

### Method 2: Tablet Compression

Tablets are produced on a Kilian Style One simulating a Fette 102i or on a Fette 102i mounted with a single set of punches, resulting in 7 mm round or 7.2×12 mm, or 7.5×13 mm, or 7.5×14 mm, or 8.4×15.5 mm oval tablets having no score. Punch size is chosen according to the total tablet weight. The press speed is set to 20 rpm. The fill volume is adjusted to obtain tablets having target weights from 107.6 to 634 mg. Compression forces around 4.5 to 14kN are applied to obtain tablets with a crushing strength from 50 to 130 N respective to the tablet size.

Prior to tablet compression the granulates obtained by method 1 are optionally blended with PCSK9 inhibitor and any additional excipients on a Turbula mixer (7 min, 25 rpm).

### Method 3: Salt Exchange

Batches of spray dried EGF(A) derivative material were dissolved in 100mM Tris buffer at neutral pH to a final concentration of 10-20 g/l. The material was subsequently loaded onto a C18 reversed-phase column up to 20 g EGF(A) per litre of resin and washed in the following order: a) with 1 column volume of a solution comprising of 5% w/w ethanol in water followed by b): 10 column volumes of a solution containing 20 mM sodium phosphate and 500 mM sodium chloride at pH 7.5 and c): 10 column volumes of a solution comprising of 5% w/w ethanol. EGF(A) was then eluted from the column by using a 50% w/w ethanol solution. Ethanol was subsequently evaporated by applying a vacuum. The solution was subsequently spray dried providing the EGF(A) derivative as a sodium salt.

### EXAMPLES

### Example 1

### Preparation of Compositions

Test compositions were prepared according to Table 1 below, comprising a peptide based PCSK9 inhibitor. The compound used is a peptide analogue of LDLR293-332 comprising two substituents in the form of fatty diacids attached via a hydrophilic linker molecule. The EGF(A) derivative is prepared as described in WO 2017/121850 (Example 151/page 161) and WO19016300. The compound is referred to as compound 151 herein and has the following structure:

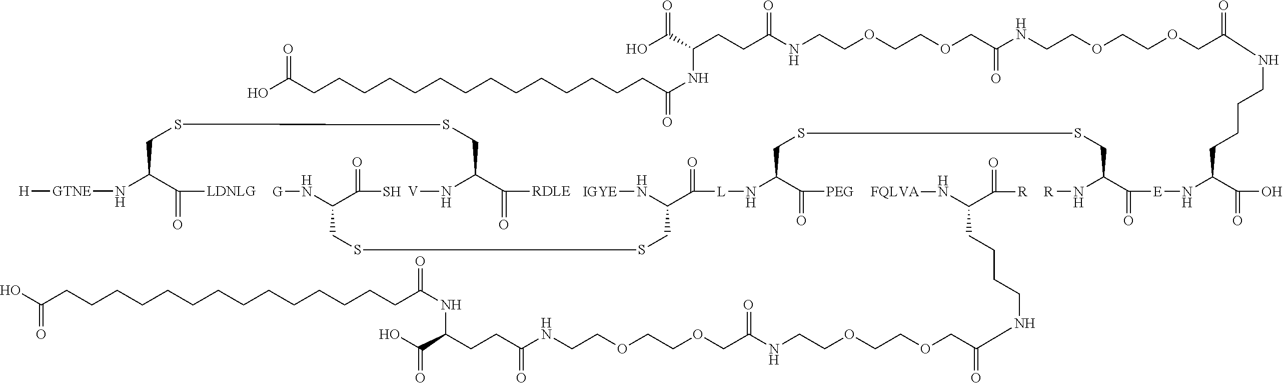

The composition was prepared by using a combination of the methods described herein above. Test composition 1 was produced by granulating a blend of SNAC, magnesium stearate and MCC as described in WO 2013/139694. The granules were subsequently blended with povidone, the PCSK9 inhibitor and further MCC and magnesium stearate prior to tablet compression (method 2). Test compositions 2 to 4 were prepared by blending of SNAC with magnesium stearate prior to dry granulation (method 1). The obtained granules where subsequently blended with PCSK9 inhibitor prior to tablet compression (method 2). Test composition 4 further included 10 mg of a GLP-1 reference molecule irrelevant for the present study. The compositions are described in Table 1.1. For test composition 1, the numbers in parenthesis provide the amounts of magnesium stearate and microcrystalline cellulose included in the SNAC granules.

TABLE 1.1
Compositions of PCSK9 inhibitor tablets

| Composition | Test 1 | Test 2 | Test 3 | Test 4 |
|---|---|---|---|---|
| SNAC (mg) | 300 | 100 | 300 | 300 |
| Magnesium stearate (mg) | 9.7 (7.7) | 2.6 | 7.7 | 7.7 |
| MCC (mg) | 80 (57) | | | |
| Compound 151 (mg) | 5 | 5 | 5 | 10 |
| Povidone (mg) | 8 | | | |

Further compositions were prepared with increasing amount of the PCSK9 inhibitor as set forth in table 1.2 below. Test compositions 5-7 were prepared using an EGF(A) derivative preparation obtained by method 3 herein, by blending of Compound 151, SNAC and magnesium stearate prior to dry granulation (method 1). The obtained granules were compressed into tablets (method 2).

TABLE 1.2
Compositions of PCSK9 inhibitor tablets

| Composition | Test 5 | Test 6 | Test 7 |
|---|---|---|---|
| SNAC (mg) | 300 | 300 | 500 |
| Magnesium stearate (mg) | 7.7 | 7.7 | 12.8 |
| Compound 151 (mg) | 50 | 100 | 100 |

### Example 2

### Disintegration Testing

The objective of the present study was to evaluate the disintegration of the series of the test compositions described in Example 1.

Disintegration was measured according to Assay II using a Pharmatech PTZ auto disintegration tester in accordance with European Pharmacopoeia employing automatic detection. Test compositions 1-6 were tested in water R and considered disintegrated when the automatic detection was deployed. The results are reported as single value or an average of 3 tablets. Table 2.1 shows the results for test compositions prepared according to Example 1 above.

TABLE 2.1
Disintegration times

| Composition | Test 1 | Test 2 | Tests | Test 5 | Test6 |
|---|---|---|---|---|---|
| Disintegration | 15 min | 4 min | 7 min | 7 min | 7 min |
| time | 18 s | 18 s | 23 s | 00 s | 38 s |

The results obtained show that the test compositions 2, 3, 4 and 5 display a significantly faster disintegration than observed for test composition 1.

### Example 3

### Dissolution Testing

The objective of the present study was to evaluate the dissolution of the series of the test compositions described in Example 1.

Dissolution was measured according to Assay III and the amount of the PCSK9 inhibitor and SNAC were measured according to Assay IV. The released amount of PCSK9 inhibitor and SNAC were calculated as percentages of the actual or nominal content in the test compositions i.e. 100 or 300 or 500 mg/tablet of SNAC and 5 or 50 or 100 mg/tablet of PCSK9 inhibitor.

The released amount of PCSK9 inhibitor is reported as single value or an average of 3 tablets.

Table 3.1 shows the results for test compositions 1, 2 and 3 prepared according to Example 1 above, wherein the release is presented as “PCSK9 inhibitor in solution (%)” describing the amount of PCSK9 inhibitor in solution after 15, 30 and 60 min relative to the total amount of PCSK9 inhibitor in the tablet at the start of the experiment. The total content of PCSK9 inhibitor and SNAC in the tablets were determined according to Assay IV.

TABLE 3.1
PCSK9 inhibitor in solution (%)

| | PCSK9 inhibitor in solution (%) | | |
|---|---|---|---|
| Composition | 15 min | 30 min | 60 min |
| Test 1 | 40.2 | 66.8 | 92.2 |
| Test 2 | 95.7 | Full release | Full release |
| Test 3 | 90.5 | Full release | Full release |

The results obtained show that the test compositions 2 and 3 display a faster release of the PCSK9 inhibitor compared to what was observed for test composition 1. A significantly faster release of the PCSK9 inhibitor was observed for the early time points, i.e. at 15 and 30 minutes. The difference in release was less significant after 60 minutes. The amount of SNAC in the test compositions did not influence the release of the PCSK9 inhibitor after 15 min, i.e. test compositions comprising 100 mg SNAC dissolve as fast as test compositions comprising 300 mg SNAC when measured after 15 min or later.

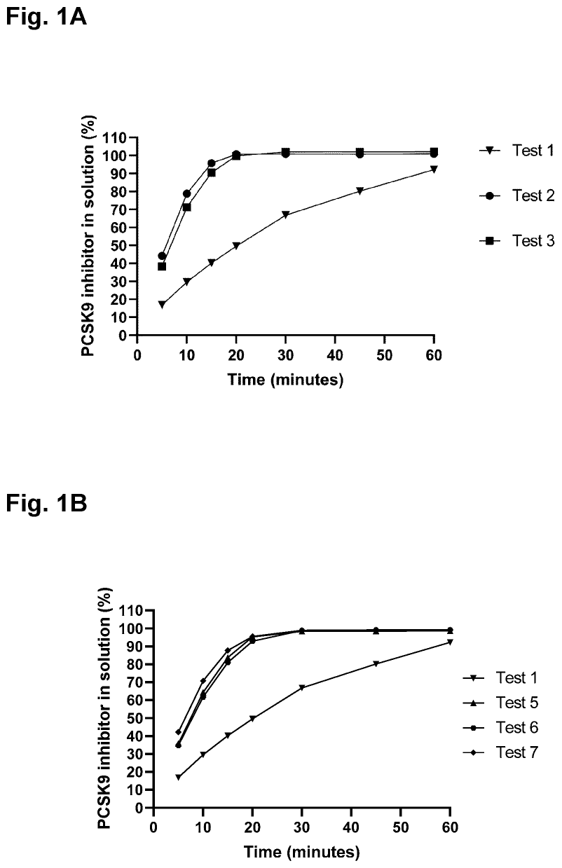

Further data obtained after 5, 10, 15, 20, 30, 45 and 60 min for test compositions 1 to 3 are shown in FIG. 1A, demonstrating that test compositions 2 and 3 are superior to test composition 1 at every time point.

As above, the dissolution of test compositions 5-7 were tested and results provided in table 3.2 and included in FIG. 1B together with test composition 1

TABLE 3.2
PCSK9 inhibitor in solution (%)

| | PCSK9 inhibitor in solution (%) | | |
|---|---|---|---|
| Composition | 15 min | 30 min | 60 min |
| Test 5 | 83.7 | Full release | Full release |
| Test 6 | 81.0 | Full release | Full release |
| Test 7 | 87.7 | Full release | Full release |

The results obtained show that the test compositions 5-7 also display a faster release of the PCSK9 inhibitor compared to what was observed for test composition 1.

### Example 4

### Pharmacokinetic (PK) Studies in Dogs

The pharmacokinetics of oral administration of the test compositions described in Example 1 above were evaluated according to Assay V to evaluate the oral exposure in beagle dogs using 10 ml water for dosing to the dogs. The number of tests performed for each formulation is indicated by n.

### Analysis and Results

The plasma concentration of the PCSK9i molecule was analysed by LCMS. Individual plasma concentration-time profiles were analysed by a non-compartmental model in WinNonlin v. 5.0 or Phoenix v. 6.2 or 6.3 (Pharsight Inc., Mountain View, Calif., USA), or other relevant software for PK analysis. The compound exposure measured at t=30 min was determined and normalized by dose /kg bodyweight.

The area under the plasma concentration versus time curve for the first 30 min (AUC, [time x concentration]) was calculated (by the Pharsight programme) after oral administration and normalized by ((dose/kg bodyweight)) to obtain the dose corrected exposure.

Plasma exposure, dose corrected exposure of PCSK9i and Cmax obtained after administration of test composition 1, 4, 6 and 7 were calculated. Data included in table 4 below, all values are expressed as geometric means.

TABLE 4
Average exposure measured in dogs after single administration of the test
compositions 1, 4, 6 and 7.

| | | Dose corrected | | | | |
|---|---|---|---|---|---|---|
| | | plasma | | | | |
| | Plasma | exposure | | Dose corrected | | |
| | exposure | t = 30 min | AUC 0-30 | AUC 0-30 min | | |
| | t = 30 min | (nmol/L)/ | min | (hr × nmol/L)/(nmol/ | Cmax | No. of |
| Composition | (nmol/L) | (nmol/kg) | (hr × nmol/L) | kg) | (nmol/L) | dogs |
| Test 1 | 9 | 0.142 | 1.6 | 0.0250 | 22 | 8 |
| (300/5) | | | | | | |
| Test 4 | 60 | 0.454 | 11.5 | 0.0874 | 150 | 5 |
| (300/10) | | | | | | |
| Test 6 | 171 | 0.122 | 31.8 | 0.0227 | 289 | 24 |
| (300/100) | | | | | | |
| Test 7 | 300 | 0.201 | 55.0 | 0.0370 | 623 | 16 |
| (500/100) | | | | | | |

An increased and accelerated exposure was observed for compositions according to the invention compared to the test composition 1.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A pharmaceutical composition comprising
a) 0.5-100 mg EGF(A) derivative,
b) 20-1000 mg of a salt of N-(8-(2-hydroxybenzoyl)amino)caprylic acid and
wherein said salt of N-(8-(2-hydroxybenzoyl)amino)caprylic acid constitutes at least 90 w/w %, such as at least 95 w/w % of the excipients of the composition.

2. The pharmaceutical composition according to claim 1, wherein the composition comprises a lubricant.

3. The pharmaceutical composition according to claim 1 consisting of:
a) an EGF(A) derivative
b) a salt of N-(8-(2-hydroxybenzoyl)amino)caprylic acid and
c) at least one lubricant.

4. The pharmaceutical composition according to claim 1, wherein said salt of N-(8-(2-hydroxybenzoyl)amino)caprylic acid constitutes at least 70 w/w % of the composition.

5. The pharmaceutical composition according to claim 1, wherein the compositions comprises 1-10 mg magnesium stearate per 100 mg of said salt of N-(8-(2-hydroxybenzoyl)amino)caprylic acid.

6. The pharmaceutical composition according to claim 1 wherein the EGF(A) derivative is selected from the group of EGF(A) derivatives; # 31, 95, 128, 133, 143, 144, 150, 151, 152 and 153 with the following structures:

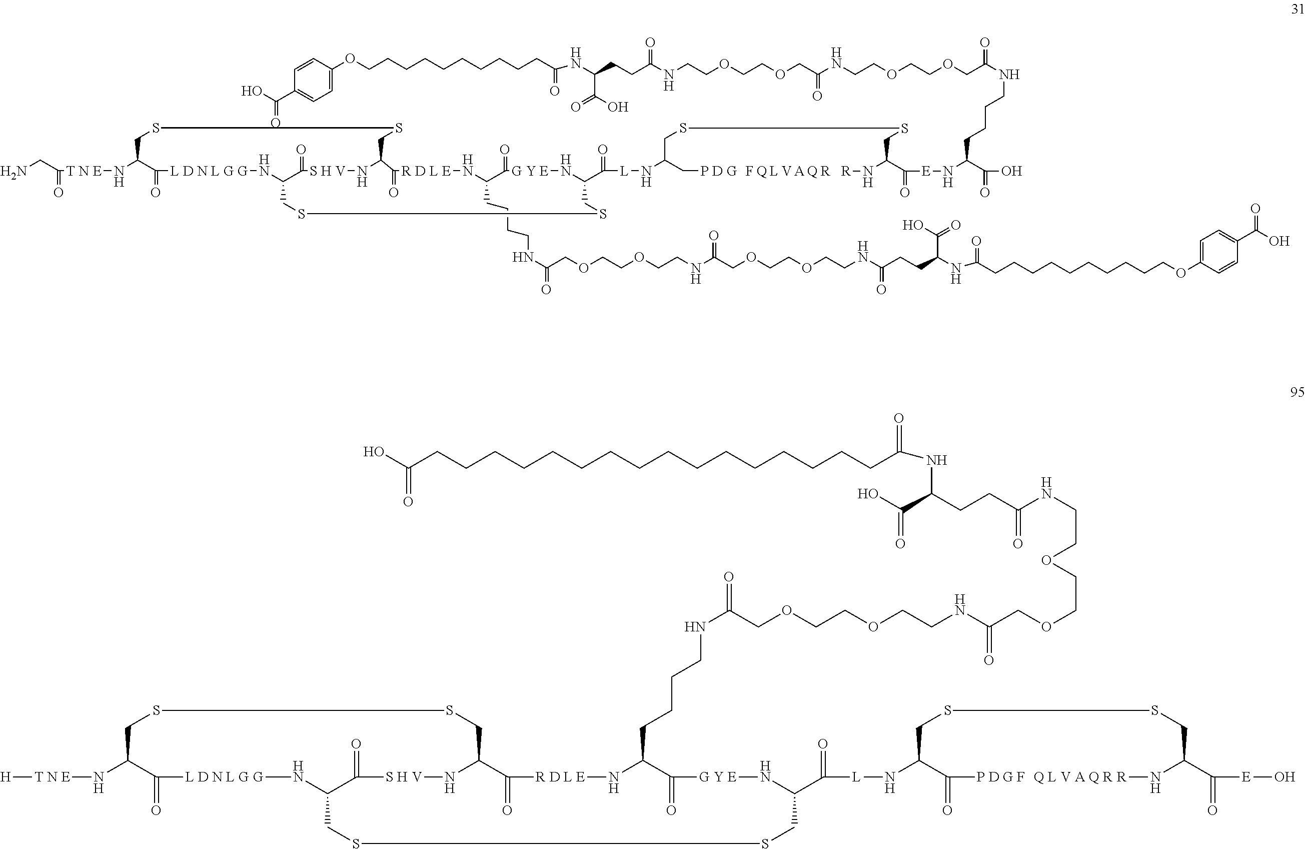

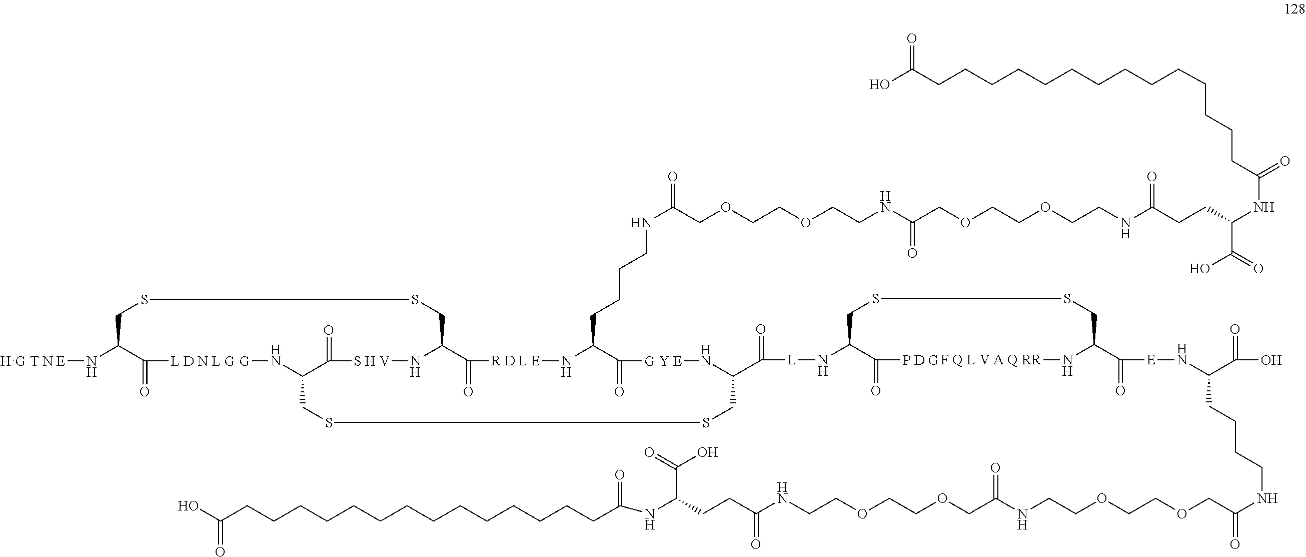

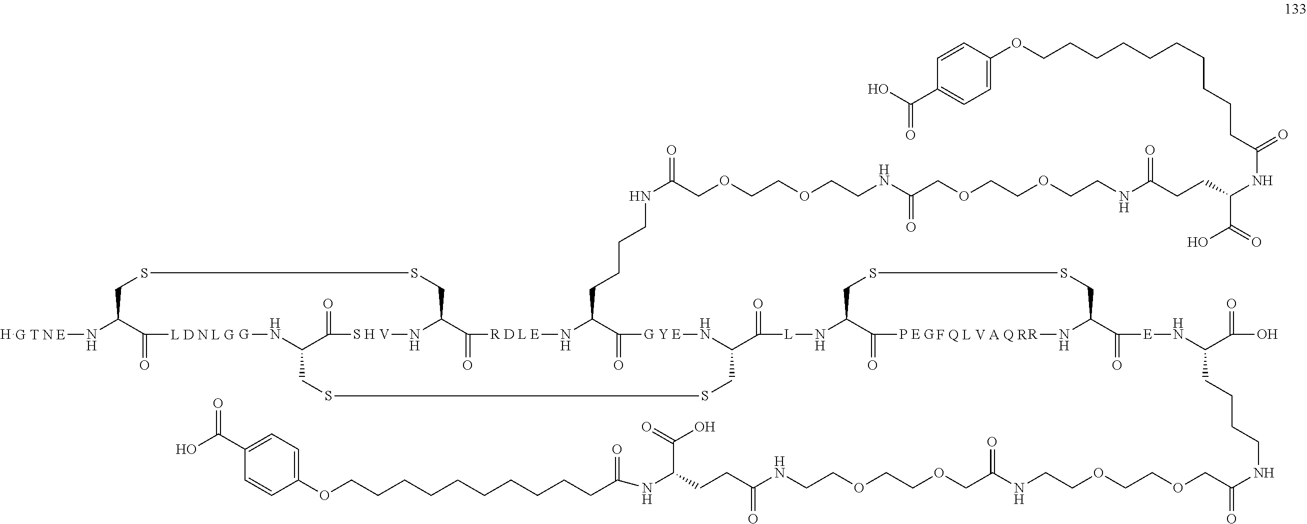

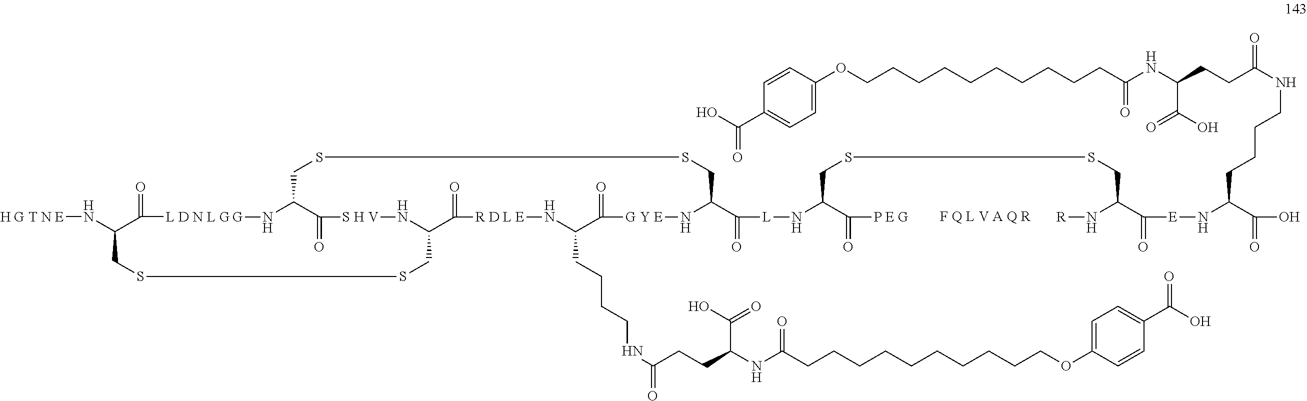

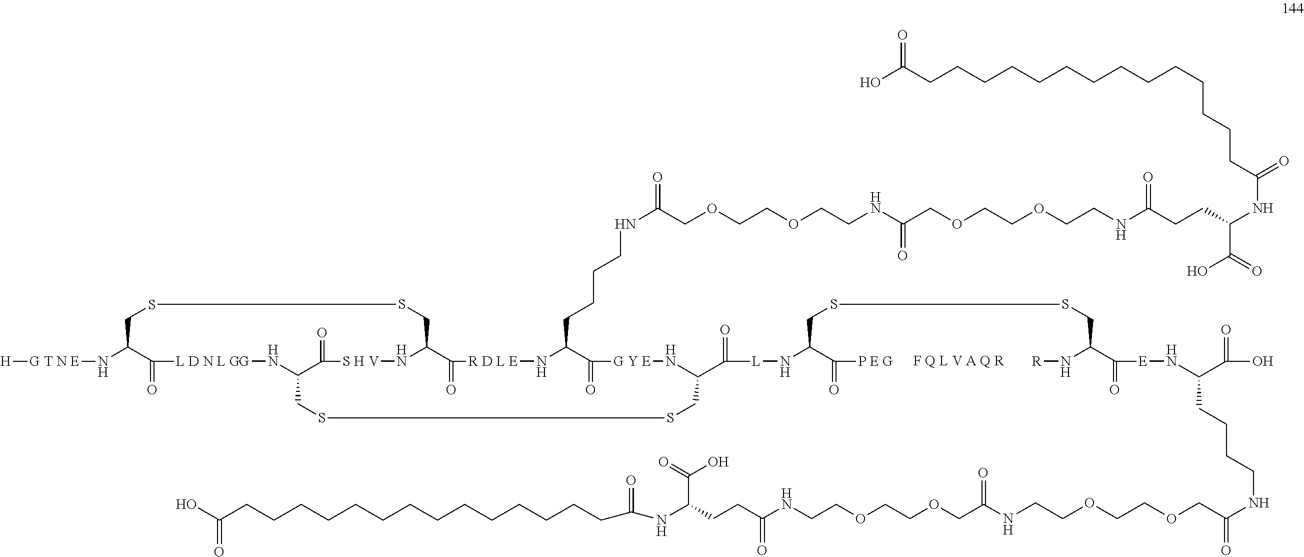

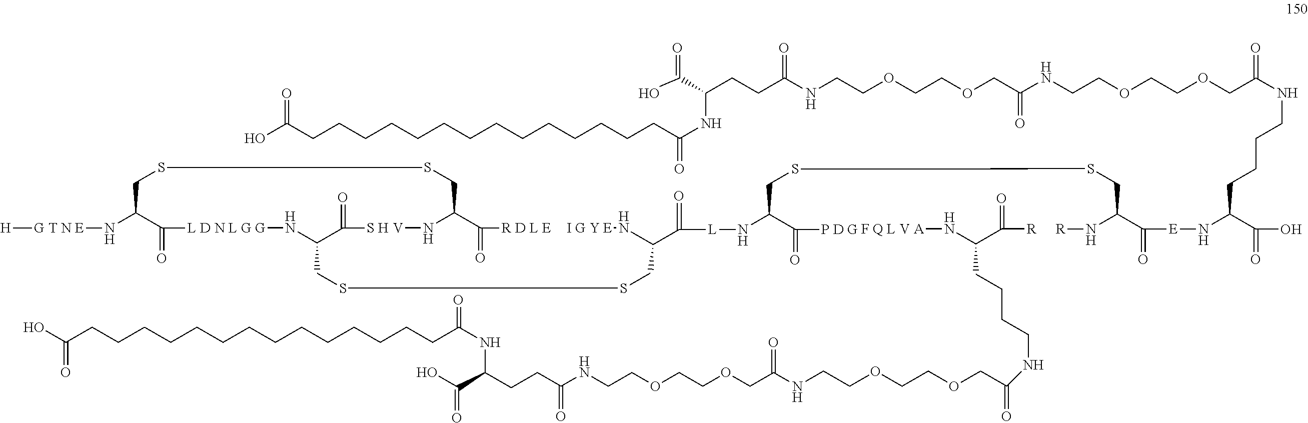

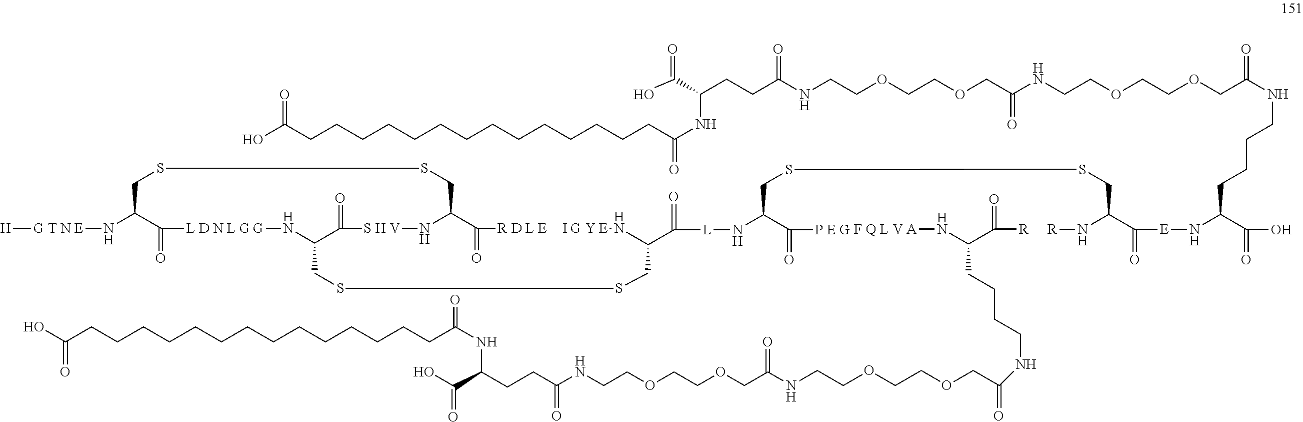

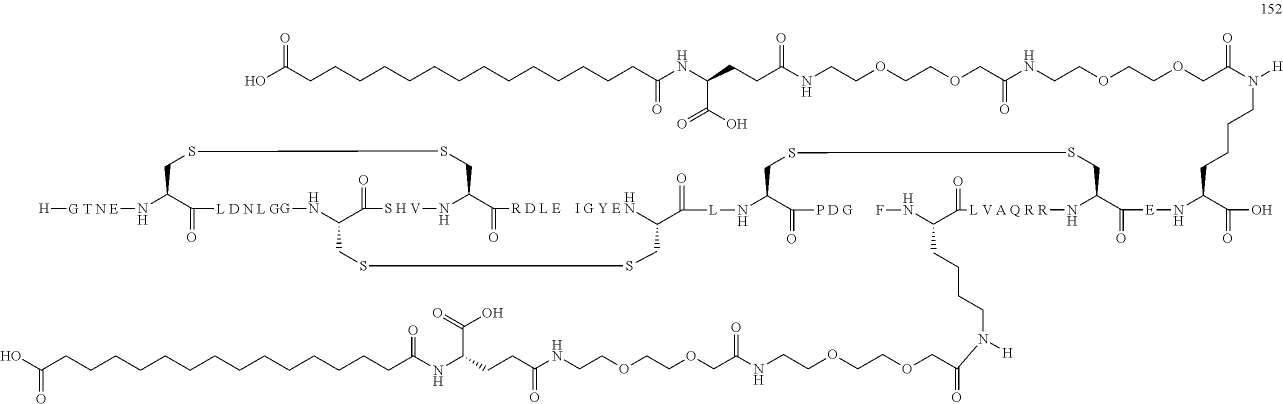

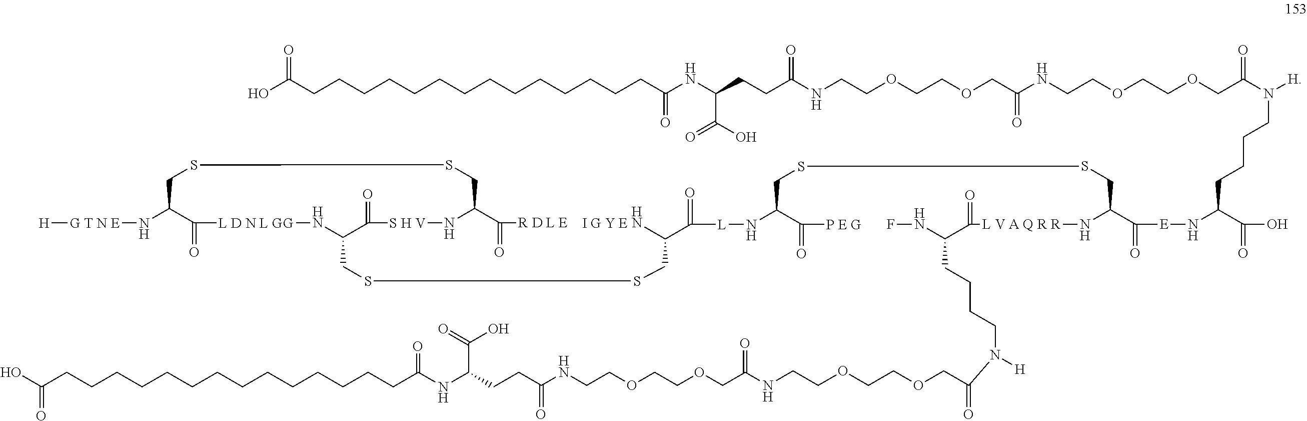

7. The pharmaceutical composition according to claim 1 any of the previous claims, wherein the salt of N-(8-(2-hydroxybenzoyl)amino)caprylic acid is sodium N-(8-(2-hydroxybenzoyl)amino)caprylate (SNAC).

8. The pharmaceutical composition according to claim 7, wherein the EGF(A) derivative is

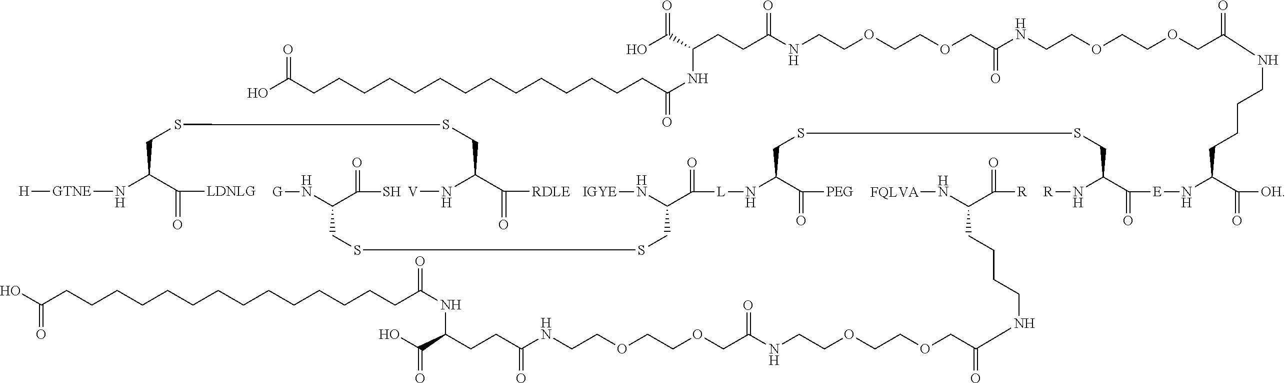

9. The pharmaceutical composition according to claim 7, wherein a dose unit comprises
a) 5-100 mg of the EGF(A) derivative,
b) 50-1000 mg of SNAC and
c) 0.5-50 mg lubricant.

10. The pharmaceutical composition according to claim 7, wherein a dose unit comprises
a) 5-100 mg of the EGF(A) derivative,
b) 100-800 mg of SNAC and
c) 1-40 mg magnesium stearate.

11. The pharmaceutical composition according to claim 8, wherein a dose unit comprises
a) 20-100 mg of the EGF(A) derivative,
b) 100-600 mg of SNAC and
c) 1-30 mg lubricant.

12. The pharmaceutical composition according to claim 8, wherein a dose unit comprises
a) 20-100 mg of the EGF(A) derivative,
b) 100-600 mg of SNAC and
c) 1-25 mg magnesium stearate per 100 mg salt of N-(8-(2-hydroxybenzoyl)amino)caprylic acid.

13. The pharmaceutical composition according to claim 1, wherein the composition is a solid composition for oral administration.

14. (canceled)

15. (canceled)

16. The pharmaceutical composition according to claim 1, comprising 50-600 mg of a salt of N-(8-(2-hydroxybenzoyl)amino)caprylic acid, wherein said salt of N-(8-(2-hydroxybenzoyl)amino)caprylic acid constitutes at least 95 w/w % of the excipients of the composition.

17. The pharmaceutical composition according to claim 2, wherein the lubricant is magnesium stearate or glyceryl dibehenate.

18. The pharmaceutical composition according to claim 2 consisting of:
a) an EGF(A) derivative
b) a salt of N-(8-(2-hydroxybenzoyl)amino)caprylic acid and
c) at least one lubricant.

19. The pharmaceutical composition according to claim 12, wherein the dose unit comprises 1-10 mg, 2-5 mg or 2-3 mg magnesium stearate per 100 mg salt of N-(8-(2-hydroxybenzoyl)amino)caprylic acid.

20. The pharmaceutical composition according to claim 13, wherein the composition is a tablet.

21. A method of improving lipid parameters and/or treating a cardiovascular disease comprising administering a pharmaceutical composition according to claim 1 to a subject in need thereof.

22. A method of improving lipid parameters and/or treating a cardiovascular disease comprising administering a pharmaceutical composition according to claim 12 to a subject in need thereof.


SEQUENCE LISTING (115 sequences)

SEQ ID NO: 1 - protein, 40 aa, Homo sapiens
GTNECLDNNGGCSHVCNDLKIGYECLCPDGFQLVAQRRCE

SEQ ID NO: 2 - protein, 40 aa, Artificial Sequence
  Synthetic sequence
GTNECLANLGGCSHICRKLKIGYECLCPDGFQLVAQRRCE

SEQ ID NO: 3 - protein, 40 aa, Artificial Sequence
  Synthetic sequence
GTNECLDNLGGCSHVCRDLKIGYECLCPDGFQLVAQRRCE

SEQ ID NO: 4 - protein, 41 aa, Artificial Sequence
  Synthetic sequence
GTNECLDNLGGCSHVCRDLEIGYECLCPDGFQLVAQRRCEK

SEQ ID NO: 5 - protein, 40 aa, Artificial Sequence
  Synthetic
GTNECLDPLGGCSHICRDLEIGYECLCPDGFQLVAQRRCE

SEQ ID NO: 6 - protein, 40 aa, Artificial Sequence
  Synthetic sequence
GTNECLDNLGGCSHVCRDLEIGYECLCPDGFQLVAQRRCE

SEQ ID NO: 7 - protein, 40 aa, Artificial Sequence
  Synthetic sequence
GTNECLKNLGGCSHVCRDLEIGYECLCPDGFQLVAQRRCE

SEQ ID NO: 8 - protein, 40 aa, Artificial Sequence
  Synthetic sequence
GTNECLDNLGGCSHVCRDLEIGYECLCPDGFQLVAQRKCE

SEQ ID NO: 9 - protein, 41 aa, Artificial Sequence
  Synthetic
NTNECLDNLGGCSHICRDLDIGYECLCPDGFQLVAQRRCEK

SEQ ID NO: 10 - protein, 41 aa, Artificial Sequence
  Synthetic sequence
NTNECLDNLGGCSHVCRDLDIGYECLCPDGFQLVAQRRCEK

SEQ ID NO: 11 - protein, 40 aa, Artificial Sequence
  Synthetic sequence
GTNECLDNLGGCSHVCRDLEIGYECLCPDGFQLVAQRRCK

SEQ ID NO: 12 - protein, 40 aa, Artificial Sequence
  Synthetic sequence
KTNECLDNLGGCSHVCRDLEIGYECLCPDGFQLVAQRRCE

SEQ ID NO: 13 - protein, 41 aa, Artificial Sequence
  Synthetic sequence
KTNECLDNLGGCSHVCRDLEIGYECLCPDGFQLVAQRRCEK

SEQ ID NO: 14 - protein, 40 aa, Artificial Sequence
  Synthetic sequence
GTNECLDNLGGCSHVCRDLEIGYECLCPDGFQLVAKHRCE

SEQ ID NO: 15 - protein, 41 aa, Artificial Sequence
  Synthetic sequence
GTNECLDNLGGCSHVCRDLEIGYECLCPDGFQLVAQRRCKK

SEQ ID NO: 16 - protein, 41 aa, Artificial Sequence
  Synthetic sequence
GTNECLDNLGGCSHVCRDLEIGYECLCPDGFQLVAQRKCEK

SEQ ID NO: 17 - protein, 41 aa, Artificial Sequence
  Synthetic sequence
GTNECLDNLGGCSHVCRDLEIGYECLCPKGFQLVAQRRCEK

SEQ ID NO: 18 - protein, 41 aa, Artificial Sequence
  Synthetic sequence
GTNECLDNLGGCSHVCRDLKIGYECLCPDGFQLVAQRRCEK

SEQ ID NO: 19 - protein, 41 aa, Artificial Sequence
  Synthetic sequence
GTNECLDNLGGCSHVCRDLEIGYECLCPEGFQLVAQRRCEK

SEQ ID NO: 20 - protein, 40 aa, Artificial Sequence
  Synthetic
GTDECLDNLGGCSHVCRDLEIGYECLCPDGFQLVAQRRCK

SEQ ID NO: 21 - protein, 40 aa, Artificial Sequence
  Synthetic sequence
GTNECLDNLGGCSHVCRDLEIGYECLCPKGFQLVAQRRCE

SEQ ID NO: 22 - protein, 40 aa, Artificial Sequence
  Synthetic sequence
GTNECLDNLGGCSHVCRDLEIGYECLCPDGFKLVAQRRCE

SEQ ID NO: 23 - protein, 40 aa, Artificial Sequence
  Synthetic sequence
GTNECLDNLGGCSHVCRDLQIGYECLCPDGFQLVAQRRCE

SEQ ID NO: 24 - protein, 40 aa, Artificial Sequence
  Synthetic sequence
GTNECLDNLGGCSHVCRDLEIGYECLCPEGFQLVAQRRCK

SEQ ID NO: 25 - protein, 40 aa, Artificial Sequence
  Synthetic sequence
KTNECLDNLGGCSHVCRDLEIGYECLCPEGFQLVAQRRCE

SEQ ID NO: 26 - protein, 40 aa, Artificial Sequence
  Synthetic sequence
GTNECLDHLGGCSHICRDLEIGYECLCPDGFQLVAQRRCE

SEQ ID NO: 27 - protein, 40 aa, Artificial Sequence
  Synthetic sequence
GTNECLDKLGGCSHVCRDLEIGYECLCPDGFQLVAQRRCE

SEQ ID NO: 28 - protein, 40 aa, Artificial Sequence
  Synthetic sequence
KKNECLDNLGGCSHVCRDLEIGYECLCPDGFQLVAQRRCE

SEQ ID NO: 29 - protein, 40 aa, Artificial Sequence
  Synthetic sequence
KTNECLDNLGGCSHVCRDLKIGYECLCPDGFQLVAQRRCE

SEQ ID NO: 30 - protein, 40 aa, Artificial Sequence
  Synthetic sequence
GTNECLDNLGGCSHVCKDLEIGYECLCPDGFQLVAQRRCE

SEQ ID NO: 31 - protein, 40 aa, Artificial Sequence
  Synthetic sequence
GTNECLDNLGGCSHVCRDLEIGYECKCPDGFQLVAQRRCE

SEQ ID NO: 32 - protein, 41 aa, Artificial Sequence
  Synthetic sequence
GTNECLDNLGGCSHVCRDLEKGYECLCPDGFQLVAQRRCEK

SEQ ID NO: 33 - protein, 40 aa, Artificial Sequence
  Synthetic sequence
GTNECLDNLGGCSHVCRDLEIGYECLCPDGFQLKAQRRCE

SEQ ID NO: 34 - protein, 40 aa, Artificial Sequence
  Synthetic sequence
GTNECLDNLGGCSHVCRDLEIGYECLCPDGFQKVAQRRCE

SEQ ID NO: 35 - protein, 40 aa, Artificial Sequence
  Synthetic sequence
GTNECLDNLGGCSHVCRDLEIGYECLCPDGKQLVAQRRCE

SEQ ID NO: 36 - protein, 40 aa, Artificial Sequence
  Synthetic sequence
GTNECLDNLGGCSHVCRDLEIGYECLCPDKFQLVAQRRCE

SEQ ID NO: 37 - protein, 40 aa, Artificial Sequence
  Synthetic sequence
GTNECLDNLGGCSHVCRDLEIGYECLCKDGFQLVAQRRCE

SEQ ID NO: 38 - protein, 40 aa, Artificial Sequence
  Synthetic sequence
GTNECLDNLGGCSHVCRDLEIGYECLCPDGFQLVAQKRCE

SEQ ID NO: 39 - protein, 40 aa, Artificial Sequence
  Synthetic sequence
GTNECLDNLGGCSHVCRDLEKGYECLCPDGFQLVAQRRCE

SEQ ID NO: 40 - protein, 40 aa, Artificial Sequence
  Synthetic sequence
GTNECLDNLGGCSHVCRDLEIGYECLCPDGFQLVAKRRCE

SEQ ID NO: 41 - protein, 40 aa, Artificial Sequence
  Synthetic sequence
GTNECLDNLGGCSHVCRDLEIGYKCLCPDGFQLVAQRRCE

SEQ ID NO: 42 - protein, 40 aa, Artificial Sequence
  Synthetic sequence
GTNECLDNLGGCSHVCRDLEIGKECLCPDGFQLVAQRRCE

SEQ ID NO: 43 - protein, 41 aa, Artificial Sequence
  Synthetic sequence
GTNECLDHLGGCSHVCRDLRIGYECLCPDGFQLVAQRRCEK

SEQ ID NO: 44 - protein, 40 aa, Artificial Sequence
  Synthetic sequence
GTNECLDNLGGCSHVCRDLEIKYECLCPDGFQLVAQRRCE

SEQ ID NO: 45 - protein, 40 aa, Artificial Sequence
  Synthetic sequence
GTNECLDNLGGCSHVCRDKEIGYECLCPDGFQLVAQRRCE

SEQ ID NO: 46 - protein, 40 aa, Artificial Sequence
  Synthetic sequence
GTNECLDNLGGCSHKCRDLEIGYECLCPDGFQLVAQRRCE

SEQ ID NO: 47 - protein, 41 aa, Artificial Sequence
  Synthetic sequence
GTNECLDNLGGCSHVCSDLRIGYECLCPDGFQLVAQRRCEK

SEQ ID NO: 48 - protein, 41 aa, Artificial Sequence
  Synthetic sequence
GTNECLDNLGGCSHVCSDLEIGYECLCPDGFQLVAQRRCEK

SEQ ID NO: 49 - protein, 40 aa, Artificial Sequence
  Synthetic sequence
GTNECLDNLGGCSYVCSDLEIGYECLCPDGFQLVAQRRCE

SEQ ID NO: 50 - protein, 40 aa, Artificial Sequence
  Synthetic sequence
NTNECLDNLGGCSHVCSDLEIGYECLCPDGFQLVAQRRCE

SEQ ID NO: 51 - protein, 40 aa, Artificial Sequence
  Synthetic sequence
GTNECLDNLGGCSKVCRDLEIGYECLCPDGFQLVAQRRCE

SEQ ID NO: 52 - protein, 40 aa, Artificial Sequence
  Synthetic sequence
GTNECLDNLGGCKHVCRDLEIGYECLCPDGFQLVAQRRCE

SEQ ID NO: 53 - protein, 40 aa, Artificial Sequence
  Synthetic sequence
GTNECLDNLGKCSHVCRDLEIGYECLCPDGFQLVAQRRCE

SEQ ID NO: 54 - protein, 40 aa, Artificial Sequence
  Synthetic sequence
GTNECLDNLKGCSHVCRDLEIGYECLCPDGFQLVAQRRCE

SEQ ID NO: 55 - protein, 41 aa, Artificial Sequence
  Synthetic sequence
NTNECLDHLGGCSHVCRDLRIGYECLCPDGFQLVAQRRCEK

SEQ ID NO: 56 - protein, 40 aa, Artificial Sequence
  Synthetic sequence
GTNECLDNKGGCSHVCRDLEIGYECLCPDGFQLVAQRRCE

SEQ ID NO: 57 - protein, 40 aa, Artificial Sequence
  Synthetic sequence
GTNECKDNLGGCSHVCRDLEIGYECLCPDGFQLVAQRRCE

SEQ ID NO: 58 - protein, 41 aa, Artificial Sequence
  Synthetic sequence
NTNECLDNLGGCSHVCRDLRIGYECLCPDGFQLVAQRRCEK

SEQ ID NO: 59 - protein, 40 aa, Artificial Sequence
  Synthetic sequence
GTNECLDNLGGCSHICNDLKIGYECLCPDGFQLVAQRRCK

SEQ ID NO: 60 - protein, 40 aa, Artificial Sequence
  Synthetic sequence
GTNECLDNLGGCSYVCNDLEIGYECLCPDGFQLVAQRRCK

SEQ ID NO: 61 - protein, 40 aa, Artificial Sequence
  Synthetic sequence
GTNECLDNLGGCSHICNDLEIGYECLCPDGFQLVAQRRCK

SEQ ID NO: 62 - protein, 40 aa, Artificial Sequence
  Synthetic sequence
GTNECLDHLGGCSHVCRDLKIGYECLCPDGFQLVAQRRCE

SEQ ID NO: 63 - protein, 40 aa, Artificial Sequence
  Synthetic sequence
GTNKCLDNLGGCSHVCRDLEIGYECLCPDGFQLVAQRRCE

SEQ ID NO: 64 - protein, 40 aa, Artificial Sequence
  Synthetic sequence
GKNECLDNLGGCSHVCRDLEIGYECLCPDGFQLVAQRRCE

SEQ ID NO: 65 - protein, 41 aa, Artificial Sequence
  Synthetic sequence
KGTNECLDNLGGCSHVCRDLEIGYECLCPDGFQLVAQRRCE

SEQ ID NO: 66 - protein, 39 aa, Artificial Sequence
  Synthetic sequence
GNECLDNLGGCSHVCRDLEIGYECLCPDGFQLVAKRRCE

SEQ ID NO: 67 - protein, 41 aa, Artificial Sequence
  Synthetic sequence
GTNECLDNLGGCSDVCRDLEIGYECLCPDGFGLVAQRRCEK

SEQ ID NO: 68 - protein, 41 aa, Artificial Sequence
  Synthetic sequence
GTNECLDNLGGCSDVCRDLEIGYECLCPDGFQLVAQRRCEK

SEQ ID NO: 69 - protein, 41 aa, Artificial Sequence
  Synthetic sequence
GTNECLDHLGGCSHVCRDLEKGYECLCPDGFQLVAQRRCEK

SEQ ID NO: 70 - protein, 40 aa, Artificial Sequence
  Synthetic sequence
GTNECLDNLGGCSHVCRDLEKGYECLCPDGFQLVAKRRCE

SEQ ID NO: 71 - protein, 40 aa, Artificial Sequence
  Synthetic sequence
GTNECLDNLGGCSHVCRDLEKGYECLCPDGFKLVAQRRCE

SEQ ID NO: 72 - protein, 41 aa, Artificial Sequence
  Synthetic sequence
GTNECLDNLGGCSHVCRDLEIGYECLCPDGFKLVAQRRCEK

SEQ ID NO: 73 - protein, 40 aa, Artificial Sequence
  Synthetic sequence
GTNECLDNLGGCSHVCRDLEKGYECLCPKGFQLVAQRRCE

SEQ ID NO: 74 - protein, 40 aa, Artificial Sequence
  Synthetic sequence
TNECLDHLGGCSHVCRDLEKGYECLCPDGFQLVAQRRCEK

SEQ ID NO: 75 - protein, 41 aa, Artificial Sequence
  Synthetic sequence
AGTNECLDNLGGCSHVCRDLEKGYECLCPDGFQLVAQRRCE

SEQ ID NO: 76 - protein, 39 aa, Artificial Sequence
  Synthetic sequence
TNECLDNLGGCSHVCRDLEKGYECLCPDGFQLVAQRRCE

SEQ ID NO: 77 - protein, 40 aa, Artificial Sequence
  Synthetic sequence
GTNECLDNLGGCSHVCRDLEKGYECLCPDGFQLVAQRRCK

SEQ ID NO: 78 - protein, 41 aa, Artificial Sequence
  Synthetic sequence
GTNECLDNLGGCSHVCRDLEIGYECLCPDGFQLVAKRRCEK

SEQ ID NO: 79 - protein, 40 aa, Artificial Sequence
  Synthetic sequence
GTNECLANLGGCSHICRDLKIGYECLCPDGFQLVAQRRCE

SEQ ID NO: 80 - protein, 40 aa, Artificial Sequence
  GTNECLDNLG GCSHVCRKLK IGYECLCPDG FQLVAQRRCE
GTNECLDNLGGCSHVCRKLKIGYECLCPDGFQLVAQRRCE

SEQ ID NO: 81 - protein, 40 aa, Artificial Sequence
  Synthetic sequence
GTNECLDNLGGCSHVCNDLKIGYECLCPDGFQLVAQRRCE

SEQ ID NO: 82 - protein, 41 aa, Artificial Sequence
  synthetic
GTNECLDHLGGCSHVCRDLEIGYECLCPDGFQLVAQRRCEK

SEQ ID NO: 83 - protein, 39 aa, Artificial Sequence
  synthetic
NECLDHLGGCSHVCRDLEKGYECLCPDGFQLVAQRRCEK

SEQ ID NO: 84 - protein, 41 aa, Artificial Sequence
  synthetic
GTNECLDNLGGCSHVCKDLEIGYECLCPDGFQLVAQRRCEK

SEQ ID NO: 85 - protein, 41 aa, Artificial Sequence
  synthetic
GTNECLDNLGGCSYVCNDLEIGYECLCPDGFKLVAQRRCEK

SEQ ID NO: 86 - protein, 41 aa, Artificial Sequence
  synthetic
GTNECLDHLGGCSHVCRDLEIKYECLCPDGFQLVAQRRCEK

SEQ ID NO: 87 - protein, 41 aa, Artificial Sequence
  synthetic
GWNECLDNLGGCSHVCRDLEIGYECLCPDGFQLVAQRRCEK

SEQ ID NO: 88 - protein, 40 aa, Artificial Sequence
  synthetic
GTNECLDNLGGCSHVCKDLEIGYECLCPDGFQLVAKRRCE

SEQ ID NO: 89 - protein, 40 aa, Artificial Sequence
  synthetic
GTNECLDNLGGCSHVCKDLEKGYECLCPDGFQLVAQRRCE

SEQ ID NO: 90 - protein, 40 aa, Artificial Sequence
  synthetic
TNECLDNLGGCSHVCRDLEIGYECLCPDGFQLVAQRRCEK

SEQ ID NO: 91 - protein, 40 aa, Artificial Sequence
  synthetic
GTNECLDNLGGCSHVCRDLEIGYECLCPDGFKLVAKRRCE

SEQ ID NO: 92 - protein, 42 aa, Artificial Sequence
  synthetic
AGTNECLDNLGGCSHVCRDLEIGYECLCPDGFQLVAQRRCEK

SEQ ID NO: 93 - protein, 41 aa, Artificial Sequence
  synthetic
GTNECLDNLGGCSYVCRDLEKGYECLCPDGFQLVAQRRCEK

SEQ ID NO: 94 - protein, 40 aa, Artificial Sequence
  synthetic
GTNECLDNLGGCSHVCKDLEIGYECLCPDGFQLVAQRRCK

SEQ ID NO: 95 - protein, 40 aa, Artificial Sequence
  synthetic
GTNECLDNLGGCSHVCRDLEIGYECLCPKGFQLVAQRRCK

SEQ ID NO: 96 - protein, 40 aa, Artificial Sequence
  synthetic
GTNECLDHLGGCSHVCRDLEKGYECLCPDGFQLVAQRRCK

SEQ ID NO: 97 - protein, 40 aa, Artificial Sequence
  synthetic
GTNECLDNLGGCSHVCRDLEKGYECLCPEGFQLVAQRRCK

SEQ ID NO: 98 - protein, 41 aa, Artificial Sequence
  synthetic
GTNECLDNLGGCSHVCRDLEKGYECLCPEGFQLVAQRRCEK

SEQ ID NO: 99 - protein, 40 aa, Artificial Sequence
  synthetic
GTNECLDNLGGCSHVCRDLEKKYECLCPDGFQLVAQRRCE

SEQ ID NO: 100 - protein, 40 aa, Artificial Sequence
  synthetic
GTNECLDNLGGCSHVCRDLKKGYECLCPDGFQLVAQRRCE

SEQ ID NO: 101 - protein, 40 aa, Artificial Sequence
  synthetic
GTNECLDNLGGCSHVCRDLKIKYECLCPDGFQLVAQRRCE

SEQ ID NO: 102 - protein, 40 aa, Artificial Sequence
  synthetic
GTNECLDNLGGCSHVCRDKEKGYECLCPDGFQLVAQRRCE

SEQ ID NO: 103 - protein, 41 aa, Artificial Sequence
  synthetic
GTNECLDHLGGCSHVCRDLEKGYECLCPEGFQLVAQRRCEK

SEQ ID NO: 104 - protein, 41 aa, Artificial Sequence
  synthetic
GTNECLDNLGGCSHVCRDLEIGYECLCPEGFQLVAKRRCEK

SEQ ID NO: 105 - protein, 41 aa, Artificial Sequence
  synthetic
GTNECLDNLGGCSHVCRDLEIGYECLCPEGFKLVAQRRCEK

SEQ ID NO: 106 - protein, 40 aa, Artificial sequence
  Synthetic
GTNECLDNLGGCSHVCKDLEIGYECLCPDGFKLVAQRRCE

SEQ ID NO: 107 - protein, 40 aa, Artificial sequence; identical to SEQ ID NO: 6
  Synthetic

SEQ ID NO: 108 - protein, 40 aa, Artificial Sequence
  Synthetic
GTNECLDNLGGCSHVCRDLEIGYECLCPEGFQLVAQRRCE

SEQ ID NO: 109 - protein, 40 aa, Artificial sequence
  Synthetic
GTNECLDNLGGCSHICRDLEIGYECLCPEGFQLVAQRRCE

SEQ ID NO: 110 - protein, 40 aa, Artificial sequence
  Synthetic
GTNECLDNLGGCSYVCNDLEIGYECLCPEGFQLVAQRRCE

SEQ ID NO: 111 - protein, 40 aa, Artificial sequence
  Synthetic
GTNECLDHLGGCSHVCRDLEIGYECLCPEGFQLVAQRRCE

SEQ ID NO: 112 - protein, 40 aa, Artificial sequence
  Synthetic
GTNECLDNLGGCSHVCRDLEKGYECLCPEGFQLVAQRRCE

SEQ ID NO: 113 - protein, 40 aa, Artificial sequence
  Synthetic
GTNECLDNLGGCSHVCRDLEIGYECLCPEGFKLVAQRRCE

SEQ ID NO: 114 - protein, 40 aa, Artificial sequence
  Synthetic
GTNECLDNLGGCSHVCRDLEIGYECLCPEGFQLVAKRRCE

SEQ ID NO: 115 - protein, 29 aa, Homo sapiens
HSQGTFTSDYSKYLDSRRAQDFVQWLMNT